(19) United States
(12) Patent Application Publication
(10) Pub. No.: US 2019/0056402 A1
(43) Pub. Date: Feb. 21, 2019

(54) ORGAN SPECIFIC DIAGNOSTIC PANELS AND METHODS FOR IDENTIFICATION OF ORGAN SPECIFIC PANEL PROTEINS

(71) Applicant: Biodesix, Inc., Boulder, CO (US)

(72) Inventors: Xiao-Jun Li, Bellevue, WA (US); Paul Kearney, Seattle, WA (US)

(21) Appl. No.: 16/042,645

(22) Filed: Jul. 23, 2018

Related U.S. Application Data
(63) Continuation of application No. 15/449,114, filed on Mar. 3, 2017, now abandoned.
Application No. 15/449,114 is a continuation of application No. 13/704,939, filed on Feb. 28, 2013, now abandoned.
(60) Provisional application No. 61/358,372, filed on Jun. 24, 2010.

(51) Int. Cl. G01N 33/574; G01N 33/68; G01N 27/62

(52) U.S. Cl. CPC G01N 33/57423; G01N 33/6842; G01N 27/62

(57) ABSTRACT

The present application provides novel compositions, methods, and assays for use in identification of appropriate diagnostic markers in blood. These compositions, methods, and assays are capable of distinguishing normal levels of detectable markers from changes in marker levels that are indicative of changes in health status.

### RELATED APPLICATIONS

This application is a continuation of U.S. application Ser. No. 15/449,114, filed on Mar. 3, 2017, which is a continuation of U.S. application Ser. No. 13/704,939, filed on Feb. 28, 2013, which is a national stage application, filed under 35 U.S.C. § 371, of PCT Application No. PCT/US2011/041887, filed on Jun. 24, 2011, which claims the benefit of U.S. Provisional Application No. 61/358,372, filed Jun. 24, 2010, the contents of each of which are incorporated by reference herein in their entireties, including drawings.

### INCORPORATION-BY-REFERENCE OF SEQUENCE LISTING

The contents of the text file named “BDR-00103 Sequence Listing.txt”, which was created on Oct. 11, 2018 and is 1.45 KB in size, are hereby incorporated by reference in their entirety.

### BACKGROUND

One aim of modern diagnostic medicine is to better identify sensitive diagnostic methods to determine changes in health status. A variety of diagnostic assays and computational methods are used to monitor health. Improved sensitivity is an important goal of diagnostic medicine. Early diagnosis and identification of disease and changes in health status may permit earlier intervention and treatment that will produce healthier and more successful outcomes for the patient. Diagnostic markers are important for assessing susceptibility to and diagnosing of disease and changes in health status. In addition, diagnostic markers are important for predicting response to treatment, determining prognosis, selecting appropriate treatment and monitoring response to treatment.

Many diagnostic markers are identified in the blood. However, identification of appropriate diagnostic markers is challenging due to the complexity and variety of detectable marker in the blood. Distinguishing between high abundance and low abundance detectable markers requires novel methods and assays to determine the differences between normal levels of detectable markers and changes of such detectable markers that are indicative of changes in health status. The present invention provides novel compositions, methods and assays to fulfill these and other needs.

### SUMMARY

According to one embodiment, a method for predicting a risk for development of a disease or change in health status is provided, the method comprising (a) obtaining a sample from a subject; (b) measuring the presence or absence of a set of sample organ specific panel proteins; (c) comparing the expression levels of the sample organ specific panel protein set to predetermined expression levels of an identical set of organ specific panel proteins from a control population; (d) determining the expression level differences between the sample organ specific panel protein set and the predetermined expression levels of the control population organ specific panel protein set; and (d) predicting a risk for development of a disease or change in health status from the expression level differences between the sample organ specific panel protein set and the control population organ specific panel protein set.

In one aspect, the sample organ specific panel proteins are measured from a target organ. In another aspect, the sample organ specific panel proteins are measured from a plurality of organs.

In one aspect, the organ specific panel protein set is selected from proteins expressed in the group of organs consisting of adrenal gland, artery, bladder, brain (amygdala), brain (nucleus caudate), breast, cervix, heart, kidney, renal cortical epithelial cells, renal proximal tubule epithelial cells, liver, hepatocytes, lung, lymph node, lymphocytes (b), lymphocytes (t), monocytes, muscle (skeletal), muscle (smooth), ovary, pancreas, pancreatic islet cells, prostate, prostate epithelial cells, skin, epidermal keratinocytes, small intestine, spleen, stomach, testes, thymus, trachea, and uterus. In another aspect, the organ specific panel protein set is selected from proteins expressed by target genes provided in Tables 1-4.

In another aspect, the organ specific panel protein set is selected such that the expression level of at least one of the organ specific panel in the sample is above or below the predetermined level. In another aspect, the expression levels of the sample organ specific panel protein set and the control population organ specific panel protein set differ by at least 10%. In another aspect, the organ specific panel protein set comprises at least five organs. In another aspect, the organ specific panel protein set comprises at least ten organs. In one aspect, the organ specific panel protein set is specific for the lung. In another aspect, the diagnostic method predicts a risk for developing lung disease.

According to another embodiment, a method for diagnosing a disease, condition or change in health status is provided, the method comprising (a) obtaining a sample of organ specific panel gene products from a subject; (b) measuring the presence or absence of a set of sample organ specific panel gene products selected from the organ specific panel genes provided in Tables 1-4; (c) comparing the levels of the set of sample organ specific panel gene products to a predetermined control range for each organ-specific gene product; and (d) diagnosing a disease, condition or change in health status based upon the difference between levels of the set of sample organ specific panel gene products and the predetermined control range for each organ specific panel gene product.

In one aspect, the biological sample is selected from the group consisting of organs, tissue, bodily fluids and cells. In another aspect, the bodily fluid is selected from the group consisting of blood, serum, plasma, urine, sputum, saliva, stool, spinal fluid, cerebral spinal fluid, lymph fluid, skin secretions, respiratory secretions, intestinal secretions, genitourinary tract secretions, tears, and milk. In another aspect, the biological sample is a blood sample.

In one aspect, the one or more organ specific panel gene products are proteins. In another aspect, the one or more organ specific panel gene products are RNA transcriptomes.

In one aspect, the disease is a lung disease. In another aspect, the lung disease is a lung cancer selected from the group consisting of small cell carcinoma, non-small cell carcinoma, squamous cell carcinoma, adenocarcinoma, broncho-alveolar carcinoma, mixed pulmonary carcinoma, malignant pleural mesothelioma and undifferentiated pulmonary carcinoma. In another aspect, the lung disease is selected from the group consisting of acute respiratory distress syndrome (ARDS), alpha-1-antitrypsin deficiency, asbestos-related lung diseases, asbestosis, asthma, bronchiectasis, bronchitis, bronchopulmonary dysplasia (BPD), chronic bronchitis, chronic obstructive pulmonary disease (COPD), congenital cystic adenomatoid malformation, cystic fibrosis, emphysema, hemothorax, idiopathic pulmonary fibrosis, infant respiratory distress syndrome, lymphangioleiomyomatosis (LAM), pleural effusion pleurisy and other pleural disorders, pneumonia, pneumonoconiosis, pulmonary arterial hypertension, pulmonary fibrosis, respiratory distress syndrome in infants, sarcoidosis and thoracentesis.

In one aspect, the set of sample organ specific panel gene products further comprises CLDN18, CPB2, WIF1, PPBP, and ALOX15B.

In one aspect, the levels of the set of sample organ specific panel gene products is determined by a method selected from the group consisting of mass spectrometry, an MRM assay, an immunoassay, an ELISA, RT-PCR, a Northern blot, and Fluorescent In Situ Hybridization (FISH). In another aspect, the levels of the set of sample organ specific panel gene products are determined by an MRM assay.

In one aspect, the diagnostic method further comprises a diagnostic kit comprising a plurality of detection reagents to detect the set of sample organ specific panel gene products. In one aspect, the plurality of detection reagents are selected from the group consisting of antibodies, capture agents, multi-ligand capture agents and aptamers.

According to another embodiment, a method for identifying a panel of disease-associated organ specific panel gene products is provided, the method comprising (a) obtaining a biological sample from a subject determined to have a disease affecting a selected organ; (b) detecting a first level of one or more organ specific panel gene products selected from any one or more of the organ specific panel genes provided in Tables 1-4 in the biological sample; (c) comparing the first level of the one or more organ specific panel gene products to a predetermined control range; and (d) selecting one or more gene products as a member of the panel of disease-associated organ specific panel gene products when the first level of one or more of the organ specific panel gene products in the biological sample is above or below the corresponding predetermined control range.

According to another embodiment, a method for generating a predetermined control range for one or more organ specific panel gene products is provided, the method comprising the steps of (a) identifying one or more organ specific panel gene products using sequencing by synthesis; (b) measuring the level of the one or more organ specific panel gene product in a set of specific healthy organs; and (c) determining a set of standard values for the one or more organ specific panel gene product that is the predetermine control range; wherein the predetermined control rage is compared to a biological sample from a subject to determine the health status of the subject.

According to another embodiment, a method for identifying a subject at risk for the development of lung cancer is provided, the method comprising (a) obtaining a sample from a subject; (b) measuring expression levels of CLDN18, CPB2, WIF1, PPBP, and ALOX15B; and (c) predicting that the subject is at risk for development of non-small cell lung cancer based upon the presence of CLDN18, CPB2, WIF1, PPBP, and ALOX15B in the sample. According to another embodiment, a method for diagnosing lung cancer is provided, the method comprising (a) obtaining a sample from a subject; (b) measuring expression levels of CLDN18, CPB2, WIF1, PPBP, and ALOX15B; and (c) predicting that the subject is at risk for development of non-small cell lung cancer based upon the expression level of CLDN18, CPB2, WIF1, PPBP, and ALOX15B in the sample.

In one aspect, the sample is a blood sample. In another aspect, the expression levels of CLDN18, CPB2, WIF1, PPBP, and ALOX15B are determined by an MRM assay.

In one embodiment, the predetermined control range is determined by analysis of a set of organs obtained by healthy tissue donors.

In one embodiment, the one or more detection reagents are specific to the first ten ranked lung cancer biomarkers in Table 4 that are in the organ of lung.

### BRIEF DESCRIPTION OF THE DRAWINGS

FIG. 1 shows a panel of five organ-specific proteins measured from different organs.

FIG. 2 is a graph illustrating the number of gene expression studies that correlated lung diseases with organ-specific proteins that relate to lung disease.

FIG. 3A and FIG. 3B are a set of graphs illustrating the median coefficient of variation (CV) as a function of maximum tag count, evaluated from replicate datasets of the same samples. FIG. 3A shows the different cDNA clones of the same samples.

FIG. 3B shows the same cDNA clones but different sequencing runs.

FIG. 4 is a cluster dendrogram of 64 sequencing-by-synthesis (SBS) datasets of various human organs.

FIG. 5 is a bar graph illustrating the specificity of a five-protein organ-specific protein panel (CLDN18, CPB2, WIF1, PPBP and ALOX15B) and the specificities of constituent proteins.

FIG. 6 is a table (Table 1) showing a list of proteins that appeared in the top 26 studies on lung diseases.

FIG. 7 is a table (Table 2) showing a list of lung-specific proteins.

FIG. 8 is a table (Table 3) showing a list of lung disease genes from top 10 studies.

FIG. 9 is a table (Table 4) showing a list of lung cancer genes from top 10 studies.

FIG. 10 is a table (Table 5) showing a summary of organ-specific proteins in different organs.

FIG. 11 is a table (Table 6) showing the information on sequencing-by-synthesis (SBS) datasets that were used for identifying organ-specific proteins.

FIG. 12 is a table (Table 7) showing a list of primer-dimers used in generating sequencing-by-synthesis (SBS) data.

FIG. 13 is a table (Table 8) showing the error rates of sequencing the last base of sequencing-by-synthesis (SBS) tags.

FIG. 14 is a table (Table 9) showing the top 50 transcriptomic studies on human diseases that has the highest correlation with lung-specific proteins (k< or =5). The most significant datasets of the studies and their corresponding p values were also listed.

### DETAILED DESCRIPTION

The present disclosure provides novel compositions, methods, assays and kits directed to diagnostic protein markers or panels of markers that are organ-specific and correlate to changes in health status or are diagnostic of a disease. The markers identified herein are sensitive and accurate diagnostic markers and directed toward specific panels of proteins that are identified in blood or tissue. The organ-specific panels are groups or sets of organ-specific panel proteins identified from organ samples obtained from populations of normal human beings and specific patient populations using the methods described herein. The present disclosure provides computational methods to identify and correlate organ-specific panel proteins and panels with disease-associated proteins. The present disclosure identifies computational methods to select the composition of organ-specific panel proteins and panels.

The organ-specific diagnostic markers of the present disclosure can be used for assessing susceptibility to and diagnosing of disease, conditions and changes in health status. In addition, the organ-specific diagnostic markers of the present disclosure are important for predicting response to and selection of treatment, monitoring treatment and determining prognosis. The organ-specific diagnostic markers may be used for staging the disease in patient (e.g., cancer) where multiple organs are involved. The organ-specific diagnostic markers may be used for monitoring the progression of the disease (e.g., lung disease). Furthermore, the markers of the present invention, alone or in combination, can be used for detection of the source of metastasis found in anatomical places other than the originating tissue. Also, one or more of the organ specific panel proteins and/or panels may be used in combination with one or more other disease markers (other than those described herein), such as conventionally defined organ-specific protein,

The diagnostic markers may optionally be determined to be used as “detection reagents”. Detection reagents, as used herein refer to any agent that that associates or binds directly or indirectly to a molecule in the sample. In certain embodiments, a detection reagent may comprise antibodies (or fragments thereof) either with a secondary detection reagent attached thereto or without, nucleic acid probes, aptamers, capture agents, or glycopeptides, etc. Further, a “panel” may comprise panels, arrays, mixtures, kits, or other arrangements of proteins, antibodies or fragments thereof to organ-specific panel proteins, nucleic acid molecules encoding organ-specific panel proteins, nucleic acid probes to that hybridize to organ-specific nucleic acid sequences or capture agents. Moreover, a panel may be derived from at least one organ or two or more organs. A panel may be derived from 3, 4, 5, 6, 7, 8, 9, 10 or more organs. The panels are comprised of a plurality of detection reagents each of which specifically detects a protein (or transcript). In most embodiments, the detection reagents are substantially organ-specific but may also comprise non-organ specific reagents for use as controls or other purposes. In certain aspects, the panels comprise detection reagents, each of which specifically detects an organ-specific protein (or transcript). The term specifically is a term of art that would be readily understood by the skilled artisan to mean, in this context, that the protein of interest is detected by the particular detection reagent but other proteins are not substantially detected. Specificity can be determined using appropriate positive and negative controls and by routinely optimizing conditions.

The organ-specific diagnostic markers of the present disclosure are unique as they are identified by computational methods that compare markers obtained from populations with specific diseases or diagnosis to a marker data set obtained from the organs of healthy cadavers. The marker data set obtained from healthy cadavers was the result of using methods described herein to identify markers from the following tissue types: adrenal gland, artery, bladder, brain (amygdala), brain (nucleus caudate), breast, cervix, heart, kidney, renal cortical epithelial cells, renal proximal tubule epithelial cells, liver, hepatocytes, lung, lymph node, lymphocytes (b), lymphocytes (t), monocytes, muscle (skeletal), muscle (smooth), ovary, pancreas, pancreatic islet cells, prostate, prostate epithelial cells, skin, epidermal keratinocytes, small intestine, spleen, stomach, testes, thymus, trachea, and uterus.

Thus, using data obtained from a normal subject population as a baseline, the disclosed methods use these data sets that include expression levels of a plurality of markers. This set of markers may include all candidate markers which may be suspected as being relevant to the detection of a particular disease, condition, or change in health status, although, actual measured relevance is not required. Embodiments of the disclosed methods may be used to determine which of the candidate markers are most relevant to the diagnosis of the disease, condition or change in health status.

Biomolecular sequences (amino acid and/or nucleic acid sequences) uncovered using the disclosed methods can be efficiently utilized as tissue or pathological markers and/or as drugs or drug targets for treating or preventing a disease. The organ-specific diagnostic markers are released to the bloodstream or are found in tissue under conditions of a particular disease, condition or change in health status. Depending upon the circumstances, the amount of released or expressed organ specific marker may be at a higher or lower level relative to normal. Similarly, when assessing the stage of a disease, condition, or change in health care status, the amount of released or expressed organ specific diagnostic marker may be at a higher or lower level relative to the level of organ specific diagnostic marker released or expressed in an individual or individuals afflicted with the same disease, condition or change in health care status. The measurement of these organ specific diagnostic markers in patient samples provides information that the clinician can correlate with the susceptibility a patient has to a particular disease, condition or health care status, a probable diagnosis of a particular disease, condition or health care status.

According to the disclosed embodiments, the terms “biomarker,” “marker,” “diagnostic marker” are interchangeable and may be an amino acid or nucleic acid sequence, including, but not limited to, DNA, RNA, microRNA, protein, peptide, or any other gene product that may be present either in blood or any other tissue or bodily fluid. The methods of the present invention may be generalized to develop diagnostic panels for any disease or health condition that utilizes DNA, RNA or protein measurements.

The terms “biomarkers,” “diagnostic markers,” “markers” and “biomolecular” sequences (amino acid and/or nucleic acid sequences) discovered using the disclosed methods can be efficiently utilized as tissue or pathological markers for diagnosing, treating or preventing a disease, condition or change in health status.

The terms “polypeptide,” “peptide,” and “protein” are used interchangeably herein to refer to an amino acid sequence comprising a polymer of amino acid residues. The terms apply to amino acid polymers in which one or more amino acid residues is an artificial chemical mimetic of a corresponding naturally occurring amino acid, as well as to naturally occurring amino acid polymers and non-naturally occurring amino acid polymers.

The terms “glycopeptide” or “glycoprotein” refers to a peptide that contains covalently bound carbohydrate. The carbohydrate can be a monosaccharide, oligosaccharide or polysaccharide. The terms “glycopeptide” or “glycoprotein” refers to a peptide that contains covalently bound carbohydrate. The carbohydrate can be a monosaccharide, oligosaccharide or polysaccharide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .γ-carboxyglutamate, and O-phosphoserine. The term “amino acid analogs” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. The term “amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “nucleic acid” or “nucleic acid sequence” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. Any products of a splicing reaction, including recombinant forms of the splice products, are included in this definition.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example, using automated oligonucleotide synthesizers that are commercially available. However, oligonucleotides can be made by a variety of other methods, including in vitro recombinant DNA-mediated techniques and by expression of DNAs in cells and organisms.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “antibody” as used herein refers to a protein of the kind that is produced by activated B cells after stimulation by an antigen and can bind specifically to the antigen promoting an immune response in biological systems. Full antibodies typically consist of four subunits including two heavy chains and two light chains. The term antibody includes natural and synthetic antibodies, including but not limited to monoclonal antibodies, polyclonal antibodies or fragments thereof. Exemplary antibodies include IgA, IgD, IgGI, IgG2, IgG3, IgM and the like. Exemplary fragments include Fab Fv, Fab′ F(ab′)2 and the like. A monoclonal antibody is an antibody that specifically binds to and is thereby defined as complementary to a single particular spatial and polar organization of another biomolecule which is termed an “epitope.” In some forms, monoclonal antibodies can also have the same structure. A polyclonal antibody refers to a mixture of different monoclonal antibodies. In some forms, polyclonal antibodies can be a mixture of monoclonal antibodies where at least two of the monoclonal antibodies binding to a different antigenic epitope. The different antigenic epitopes can be on the same target, different targets, or a combination. Antibodies can be prepared by techniques that are well known in the art, such as immunization of a host and collection of sera (polyclonal) or by preparing continuous hybridoma cell lines and collecting the secreted protein (monoclonal).

The term “aptamers” as used here indicates oligonucleic acid or peptide molecules that bind a specific target. In particular, nucleic acid aptamers can comprise, for example, nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers are useful in biotechnological and therapeutic applications as they offer molecular recognition properties that rival that of the antibodies.

The term “multi-ligand capture agents” used herein indicates an agent that can specifically bind to a target through the specific binding of multiple ligands comprised in the agent. For example, a multi-ligand capture agent can be a capture agent that is configured to specifically bind to a target through the specific binding of multiple ligands comprised in the capture agents. Multi-ligand capture agents can include molecules of various chemical natures (e.g., polypeptides polynucleotides and/or small molecules) and comprise both capture agents that are formed by the ligands and capture agents that attach at least one of the ligands.

In particular, multi-ligand capture agents herein described can comprise two or more ligands each capable of binding a target. The term “ligand” as used herein indicates a compound with an affinity to bind to a target. This affinity can take any form. For example, such affinity can be described in terms of non-covalent interactions, such as the type of binding that occurs in enzymes that are specific for certain substrates and is detectable. Typically, those interactions include several weak interactions, such as hydrophobic, van der Waals, and hydrogen bonding which typically take place simultaneously. Exemplary ligands include molecules comprised of multiple subunits taken from the group of amino acids, non-natural amino acids, and artificial amino acids, and organic molecules, each having a measurable affinity for a specific target (e.g., a protein target). More particularly, exemplary ligands include polypeptides and peptides, or other molecules which can possibly be modified to include one or more functional groups. The disclosed ligands, for example, can have an affinity for a target, can bind to a target, can specifically bind to a target, and/or can be bindingly distinguishable from one or more other ligands in binding to a target. Generally, the disclosed multi-ligand capture agents will bind specifically to a target. Where it is not necessary that the individual ligands comprised in the multi-ligand capture agent be capable of specifically binding to the target individually, although this is also contemplated.

### Diagnostic Assays

In some embodiments, the biomarkers are present in tissues and/or organs at normal physiological conditions, but when expressed at a higher or lower level in tissue or cells are indicative of a disease, condition or change in health status. In other embodiments, the biomarkers may be absent in tissues and/or organs under normal physiological conditions, but when expressed in tissue or cells, are indicative of a disease, condition or change in health status. In other embodiments, the biomarkers may be specifically released to the bloodstream by changes in health, or diseases, and/or are over- or under-expressed as compared to normal levels. Measurement of biomarkers in patient samples provides information that may correlate with a diagnosis of a selected disease. In one embodiment, the disease is a lung disease or lung cancer.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the disclosed methods can be affected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” (patient) may also optionally comprise a sample that has not been physically removed from the subject, as described in greater detail below.

In some embodiments, the disclosed methods provide for obtaining a sample from a subject or a patient. As used herein, the term “subject” refers to any animal (e.g., a mammal), including but not limited to humans, non-human primates, rodents, dogs, pigs, and the like. In certain embodiments, it is contemplated that one or more cells, tissues, or organs are separated from an organism. The term “isolated” can be used to describe such biological matter. It is contemplated that the methods of the present invention may be practiced on in vivo and/or isolated biological matter.

Though tissue is composed of cells, it will be understood that the term “tissue” refers to an aggregate of similar cells forming a definite kind of structural material. Moreover, an organ is a particular type of tissue. The term “organ” refers to any anatomical part or member having a specific function in the animal. Further included within the meaning of this term are substantial portions of organs (e.g., cohesive tissues obtained from an organ). Such organs include but are not limited to kidney, liver, heart, skin, large or small intestine, pancreas, and lungs. Further included in this definition are bones and blood vessels (e.g., aortic transplants).

In certain embodiments, the tissue or organ is “isolated,” meaning that it is not located within an organism.

Examples of suitable biological samples which may optionally be used with preferred embodiments of the present invention include but are not limited to blood, serum, plasma, blood cells, urine, sputum, saliva, stool, spinal fluid or CSF, lymph fluid, the external secretions of the skin, respiratory, intestinal, and genitourinary tracts, tears, milk, neuronal tissue, lung tissue, any human organs or tissue, including any tumor or normal tissue, any sample obtained by lavage (for example of the bronchial system or of the breast ductal system), and also samples of in vivo cell culture constituents. In a preferred embodiment, the biological sample comprises lung tissue and/or sputum and/or a serum sample and/or a urine sample and/or any other tissue or liquid sample. The sample can optionally be diluted with a suitable eluant before contacting the sample to an antibody and/or performing any other diagnostic assay.

Numerous well known tissue or fluid collection methods can be utilized to collect a biological sample from a subject in order to determine the level of DNA, RNA and/or polypeptide of the variant of interest in the subject. Examples include, but are not limited to, fine needle biopsy, needle biopsy, core needle biopsy and surgical biopsy (e.g., brain biopsy), and lavage. Regardless of the procedure employed, once a biopsy/sample is obtained the level of the diagnostic marker can be determined and a diagnosis can thus be made.

As used herein, the term “level” refers to expression levels of RNA and/or protein and/or DNA copy number of a marker of the present invention. Determining the level of the same marker in normal tissues of the same origin is used as a comparison to detect an elevated expression and/or amplification and/or a decreased expression, of the marker compared to the normal tissues. Typically the level of the marker in a biological sample obtained from the subject is different (i.e., increased or decreased) from the level of the same marker in a similar sample obtained from a healthy individual (examples of biological samples are described herein).

A “test sample” or “test amount” of a marker refers to an amount of a marker in a subject's sample that is consistent with a diagnosis a disease, condition or change in health status. In one embodiment, the disease is lung cancer. A test sample or test amount can be either in absolute amount (e.g., nanogram/mL or microgram/mL) or a relative amount (e.g., relative intensity of signals).

A “control sample” or “control amount” of a marker can be any amount or a range of amounts to be compared against a test amount of a marker. For example, a control amount of a marker can be the amount of a marker in a population of patients with a specified disease (or one of the above indicative conditions) or a control population of individuals without said disease (or one of the above indicative conditions). A control amount can be either in absolute amount (e.g., nanogram/mL or microgram/mL) or a relative amount (e.g., relative intensity of signals).

An “increase or a decrease” in the level of a gene product compared to a preselected control level as used herein refers to a positive or negative change in amount from the control level. An increase is typically at least 10%, or at least 20%, or 50%, or 2-fold, or at least 2-fold, 3-fold, 4, fold, 5-fold, to at least 10-fold to at least 20-fold to at least 40 fold or higher. Similarly, a decrease is typically at a similar fold difference or at least 10%, 20%, 30%, 40% at least 50%, or at least 80%, or at least 90%, or even as high as more than 99% in reduction from the control level.

The terms “differentially expressed gene,” “differential gene expression” and their synonyms, which are used interchangeably, refer to a gene whose expression is activated to a higher or lower level in a subject suffering from a disease, a condition or change in health status relative to its expression in a normal population or control population. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about two-fold, or at least 2-fold, 3-fold, 4, fold, 5-fold, to at least 10-fold to at least 20-fold to at least 40 fold or higher. Similarly, a difference between the expression of a given gene in normal and diseased subjects, or in various stages of disease development in a diseased subject. Differential gene expression may also be described as a percentage change when a subject is compared typically at a similar fold difference or at least 10%, 20%, 30%, 40% at least 50%, or at least 80%, or at least 90%, or even as high as more than 99% in reduction from the control level.

In one example, described herein, the organ specific diagnostic markers may be used for staging a lung disease or a lung cancer and/or monitoring the progression of the disease or cancer. Further, one or more of the organ specific diagnostic markers may optionally be used in combination with one or more other lung disease or lung cancer biomarkers (other than those described herein).

The phrase “differentially present” refers to differences in the quantity of a marker present in a sample taken from patients having a disease or one of the above indicative conditions) as compared to a comparable sample taken from patients who do not have a disease or one of the above indicative conditions. For example, a nucleic acid fragment may be differentially present between the two samples if the amount of the nucleic acid fragment in one sample is significantly different from the amount of the nucleic acid fragment in the other sample, for example as measured by hybridization and/or NAT-based assays which involve nucleic acid amplification technology, such as PCR for example (or variations thereof such as real-time PCR for example). A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present.

The terms “cancer” and “cancerous” refer to or describe the physiological condition in mammals that is typically characterized by unregulated cell growth. Examples of cancer include but are not limited to, breast cancer, colon cancer, rectal cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, esophageal cancer, testicular cancer, uterine cancer, brain cancer, lymphoma, sarcomas and leukemia.

In one embodiment, the disease is a lung cancer. In another embodiment, the disease is a lung disease.

A lung cancer as described herein may include, but is not limited to, small cell carcinoma, non-small cell carcinoma, squamous cell carcinoma, adenocarcinoma, broncho-alveolar carcinoma, mixed pulmonary carcinoma, malignant pleural mesothelioma or undifferentiated pulmonary carcinoma.

A lung disease as described herein may include, but is not limited to, acute respiratory distress syndrome (ARDS), alpha-1-antitrypsin deficiency, acute respiratory distress syndrome (ARDS), asbestos-related lung diseases, asbestosis, asthma, bronchiectasis, bronchitis, bronchopulmonary dysplasia (BPD), chronic bronchitis, chronic obstructive pulmonary disease (COPD), congenital cystic adenomatoid malformation, cystic fibrosis, emphysema, hemothorax, idiopathic pulmonary fibrosis, infant respiratory distress syndrome, lymphangioleiomyomatosis (LAM), pleural effusion pleurisy and other pleural disorders, pneumonia, pneumonoconiosis, pulmonary arterial hypertension, pulmonary fibrosis, respiratory distress syndrome in infants, sarcoidosis or thoracentesis.

The “pathology” of (tumor) cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

### Computational Methods for Diagnosis, Prognosis and Otherwise Monitoring a Disease

The embodiments provided herein are also be directed to a computational method or algorithm used for prognosis, prediction, screening, early diagnosis, staging, therapy selection and treatment monitoring of any selected disease, condition or change in health status. Such a method is based on (1) identification of organ-specific gene products and/or panels, (2) assigning a weight to the organ-specific gene products and/or panels to reflect their value in prognosis, prediction, screening, early diagnosis, staging, therapy selection and treatment monitoring a particular disease, and (3) determination of threshold values used to divide patients into groups with varying degrees of risk. Such methods are described in detail in the examples below.

The first step in generating data to be analyzed by the algorithm is gene or protein expression profiling. In some embodiments, an assay issued to detect and measure the levels of specified genes (mRNAs) or their expression products (proteins) in a biological sample comprising cancer cells.

### Identification of Organ-Specific Panel Gene Products

According to the embodiments described herein, organ-specific panel proteins and organ-specific panels are provided. Previous methods have defined a protein (or other gene product) as being organ-specific if the majority (50% or more) of its expression level across the organs and/or tissues of the human body (or some other species) is from one organ [2, 5, 6, 9]. For example, if the expression level of a protein across 25 human organs was measured and greater than 50% of that expression was in the kidney then the protein would be considered kidney-specific.

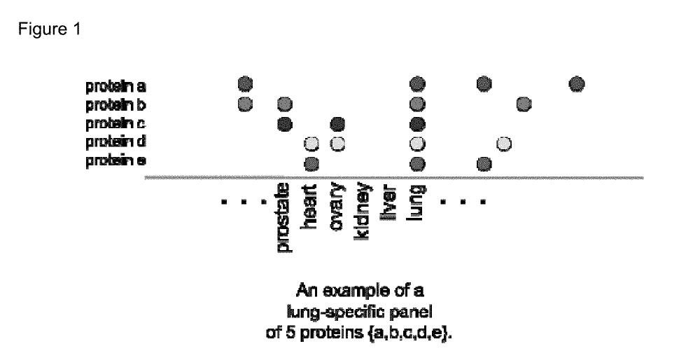

An organ-specific panel protein is a protein whose expression level across a set or group of organs and/or tissues of the human body (or some other species) is predominately (50% or more) from a fixed number (k) or fewer organs where k is some predefined number such as 5 (FIG. 1). For example, if the expression level of a protein across 25 human organs was measured and 90% of that expression was in k or fewer organs (e.g., kidney, liver, lung, bladder and spleen), then the protein would be considered {kidney, liver, lung, bladder, spleen}-specific. Equivalently, it would be considered kidney-specific (and liver-specific, lung-specific, bladder-specific and spleen-specific). This generalization is motivated by the fact that diagnostics are becoming increasingly multivariate (i.e., measuring multiple analytes such as proteins or genes) so that a multivariate definition of organ-specificity is required. For purposes of this invention, k organs refers to any number of the organs from the following exemplary tissue types: adrenal gland, artery, bladder, brain (amygdala), brain (nucleus caudate), breast, cervix, heart, kidney, renal cortical epithelial cells, renal proximal tubule epithelial cells, liver, hepatocytes, lung, lymph node, lymphocytes (b), lymphocytes (t), monocytes, muscle (skeletal), muscle (smooth), ovary, pancreas, pancreatic islet cells, prostate, prostate epithelial cells, skin, epidermal keratinocytes, small intestine, spleen, stomach, testes, thymus, trachea, and uterus. Thus k may be from 1 to 5, to 10, to 20, to 25 to 25 to 30 organs or tissue types.

To evaluate whether a protein is an organ-specific panel protein, the following analysis is used. First, the protein's abundance in different organs was sorted from high to low. More specifically, the SBS tag counts of the protein were sorted such that n₁≥n₂≥ . . . ≥n₂₅, where nᵢ was the tag count in organ. The protein is specific to the first k organs if its tag counts satisfy all three conditions listed below:
1. Tag counts in the first k organs were at or above the noise level of SBS data while those in other organs were below the noise level, i.e., nₖ≥10 and nₖ₊₁<10;
2. Tag counts in the first k organs were significantly above those in other organs. We used an exact binomial test to calculate the p value distinguishing the drawing of nₖ tags from a total of S₂₅ tags with the drawing of nₖ₊₁ tags from S₂₅ tags, where S₂₅ was the total tag count in all organs. The difference was considered significant if the two-sided p value was no greater than 0.05;
3. The total tag count in the first k organs was at least half of the total in all organs, i.e., Sₖ/S₂₅≥0.5, where Sₖ was the total tag count in the first k organs.

A panel of n organ-specific panel proteins is organ-specific if there is an organ in which all n organ-specific panel proteins, individually, are expressed. Although the term “protein” is used to describe organ-specific panels herein, this definition applies to all suitable gene products, including nucleic acid molecules and proteins and functional fragments thereof. The term ‘protein’ is used for convenience.

More generally, every protein has an expression profile across a library of organs and/or tissues. If p denotes the protein then let e(p) denote the expression profile across organs and/or tissues. Furthermore, assume e(p) is normalized so that e(p) represents a probability distribution, that is, the sum of e(p) across all organs/tissues is 1. Let S be a panel of n proteins, namely, {p1, p2, . . . , pn}. The joint probability distribution of S across the organs/tissues is simply e(S)=C*e(p1)*e(p2)* . . . *e(pn) where C is a constant normalization factor so that the sum of e(S) across all organs/tissues is 1. Finally, let T be a percentage threshold, e.g., 80%, that defines organ-specificity for a panel. The S is organ-specific for an organ Q if the probability of Q is T or greater in e(S) and all other organs have probability below T.

The organ-specific panel proteins and panels described herein may be associated with known disease-associated proteins. We used the NextBio database obtained from NextBio, Inc. (Cupertino, Calif.) to compare the population of markers obtained from the healthy cadaver donors with markers defined in various clinical studies related to lung disease and lung cancer. However, the computational methods of the present invention may be generalized to any disease process. As described in the examples below, 115 novel lung-specific proteins (k=5) were identified and compared to the NextBio clinical study database which associates a list of proteins (115) to clinical studies containing a statistically significant subset of these proteins (or their gene origins) where these proteins are modulated by disease. This enables the identification of proteins that are both organ-specific and disease modulated. Such panels of proteins are then more specific to an organ (and its diseases) than non-organ-specific panels. (see Table 2).

The 115 lung-specific proteins identified in Example 2 (Tables 2 and 5) were compared with disease-relevant genes in the NextBio studies. As anticipated, it was found that traditionally defined lung-specific proteins were highly indicative of lung diseases and lung cancers. Unexpectedly, we discovered that proteins that were not traditionally defined as lung specific were also highly correlated with lung diseases and lung cancers. These proteins are organ-specific panel proteins, more specifically, lung-specific panel proteins according to the present invention. Two sets of these lung-specific proteins that had high potential to be biomarkers for lung diseases or lung cancers were also identified. In one analysis, we determined that a five-protein lung-specific panel of proteins according to the present invention were biomarkers for lung cancer as set forth in the below examples. The five-protein panel demonstrated that the panel was both lung-specific and highly indicative for lung cancers even though the proteins were not entirely lung-specific according to the traditional definition of an organ specific protein.

### Methods of Measuring Protein Diagnostic Markers

There are a variety of methods used to measure protein diagnostic markers. As anyone skilled in the art will determine, typical methods that measure changes in mRNA expression may be used to determine control and test levels of proteins.

Methods of gene expression profiling directed to measuring mRNA levels can be divided into two large groups: methods based on hybridization analysis of polynucleotides, and methods based on sequencing of polynucleotides. The most commonly used methods known in the art for the quantification of mRNA expression in a sample include northern blotting and in situ hybridization (Parker & Barnes, Methods in Molecular Biology 106:247-283 (1999)); RNAse protection assays (Hood, Biotechniques 13:852-854 (1992)); and reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

RNA sequencing (“Whole Transcriptome Shotgun Sequencing” (“WTSS”)) will be used in transcriptomics and refers to the use of high-throughput sequencing technologies to sequence cDNA to get information about a sample's RNA content, and is used in the study of diseases like cancer.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). While the practice of the invention will be illustrated with reference to techniques developed to determine mRNA levels in a biological (e.g., tissue) sample, other techniques, such as methods of proteomics analysis are also included within the broad definition of gene expression profiling, and are within the scope herein. In general, a preferred gene expression profiling method for use with paraffin-embedded tissue is quantitative reverse transcriptase polymerase chain reaction (qRT-PCR), however, other technology platforms, including mass spectroscopy and DNA microarrays can also be used.

A sensitive and flexible quantitative method is reverse transcriptase PCR (RT-PCR), which can be used to compare mRNA levels in different sample populations, in normal and tumor tissues, with or without drug treatment, to characterize patterns of gene expression, to discriminate between closely related mRNAs, and to analyze RNA structure. A variation of the RT-PCR technique is the real time quantitative PCR (qRT-PCR), which measures PCR product accumulation through a dual-labeled fluorigenic probe (i.e., TaqMan® probe). Real time PCR is compatible both with quantitative competitive PCR, where an internal competitor for each target sequence is used for normalization, and with quantitative comparative PCR using a normalization gene contained within the sample, or a housekeeping gene for RT-PCR. For further details see, e.g., Held et al., Genome Research 6:986-994 (1996).

Differential gene expression can also be identified, or confirmed using the microarray technique. In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GeneChip® or other suitable microarray technology.

In some embodiments, genomic sequence analysis, or genotyping, may be performed on the sample. This genotyping may take the form of mutational analysis such as single nucleotide polymorphism (SNP) analysis, insertion deletion polymorphism (InDel) analysis, variable number of tandem repeat (VNTR) analysis, copy number variation (CNV) analysis or partial or whole genome sequencing. Methods for performing genomic analyses are known to the art and may include high throughput sequencing. Methods for performing genomic analyses may also include microarray methods as described. In some cases, genomic analysis may be performed in combination with any of the other methods herein. For example, a sample may be obtained, tested for adequacy, and divided into aliquots. One or more aliquots may then be used for cytological analysis of the present invention, one or more may be used for RNA expression profiling methods of the present invention, and one or more can be used for genomic analysis. It is further understood the present invention anticipates that one skilled in the art may wish to perform other analyses on the biological sample that are not explicitly provided herein.

Serial analysis of gene expression (SAGE) is a method that allows the simultaneous and quantitative analysis of a large number of gene transcripts, without the need of providing an individual hybridization probe for each transcript. For more details see, e.g., Velculescu et al., Science 270:484-487 (1995); and Velculescu et al., Cell 88:243-51 (1997).

Gene expression analysis by massively parallel signature sequencing (MPSS), described by Brenner et al., Nature Biotechnology 18:630-634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads per cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Immunoassays.

An “immunoassay” is an assay that uses an antibody to specifically bind an antigen. The immunoassay is characterized by the use of specific binding properties of a particular antibody to isolate, target, and/or quantify the antigen.

For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Immunohistochemistry.

Immunohistochemistry methods are also suitable for detecting the expression levels of the prognostic biomarkers described herein. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Proteomics.

The term “proteome” is defined as the totality of the proteins present in a sample (e.g., organ, tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g., by mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

Transcriptome.

The term “transcriptome” is defined as the totality of RNA transcripts present in a sample (e.g., organ, tissue, organism, population of cells or a single cell) at a certain point of time. Transcriptomics includes, among other things, study of the global changes of RNA transcripts present in a sample.

Mass Spectrometry Methods.

The use of mass spectrometry, in accordance with the disclosed methods and organ specific panels can provide information on not only the mass to charge ratio of ions generated from a sample, but also the relative abundance of such ions. Under standardized experimental conditions, it is therefore possible to compare the abundance of a noncovalent biomolecule-ligand complex ion with the ion abundance of the noncovalent complex formed between a biomolecule and a standard molecule, such as a known substrate or inhibitor. Through this comparison, binding affinity of the ligand for the biomolecule, relative to the known binding of a standard molecule, may be ascertained. In addition, the absolute binding affinity can also be determined.

A variety of mass spectrometry systems can be employed for identifying and/or quantifying organ-specific proteins in biological samples. Mass analyzers with high mass accuracy, high sensitivity and high resolution include, but are not limited to, ion trap, triple quadrupole, and time-of-flight, quadrupole time-of-flight mass spectrometers and Fourier transform ion cyclotron mass analyzers (FT-ICR-MS). Mass spectrometers are typically equipped with matrix-assisted laser desorption (MALDI) and electrospray ionization (ESI) sources, although other methods of peptide ionization can also be used. In ion trap MS, analytes are ionized by ESI or MALDI and then put into an ion trap. Trapped ions can then be separately analyzed by MS upon selective release from the ion trap. Organ-specific proteins can be analyzed, for example, by single stage mass spectrometry with a MALDI-TOF or ESI-TOF system.

Mass spectrometry may be used to detect proteins in a biological sample. MS relies on the discriminating power of mass analyzers to select a specific analyte and on ion current measurements for quantitation. In the field of analytical chemistry, many small molecule analytes (e.g., drug metabolites, hormones, protein degradation products and pesticides) are routinely measured using this approach at high throughput with great precision (CV<5%). Most such assays employ electrospray ionization followed by two stages of mass selection: a first stage (MS1) selecting the mass of the intact analyte (parent ion) and, after fragmentation of the parent by collision with gas atoms, a second stage (MS2) selecting a specific fragment of the parent, collectively generating a selected reaction monitoring (SRM, plural MRM) assay. The two mass filters produce a very specific and sensitive response for the selected analyte, which can be used to detect and integrate a peak in a simple one-dimensional chromatographic separation of the sample. In principle, this MS-based approach can provide absolute structural specificity for the analyte, and, in combination with appropriate stable-isotope labeled internal standards (SIS), it can provide absolute quantitation of analyte concentration. These measurements have been multiplexed to provide 30 or more specific assays in one run. Such methods are slowly gaining acceptance in the clinical laboratory for the routine measurement of endogenous metabolites (e.g., in screening newborns for a panel of inborn errors of metabolism) and some drugs (e.g., immunosuppresants).

Thus, in some embodiments, the mass spectrometry assay may include a multiple reaction monitoring (MRM) assay may be used. An MRM approach may be applied to the measurement of specific peptides in complex mixtures such as tryptic digests of plasma. In this case, a specific tryptic peptide can be selected as a stoichiometric representative of the protein from which it is cleaved, and quantitated against a spiked internal standard (a synthetic stable-isotope labeled peptide) to yield a measure of protein concentration. In principle, such an assay requires only knowledge of the masses of the selected peptide and its fragment ions, and an ability to make the stable isotope-labeled version. C-reactive protein, apo A-I lipoprotein, human growth hormone and prostate-specific antigen (PSA) have been measured in plasma or serum using this approach. Since the sensitivity of these assays is limited by mass spectrometer dynamic range and by the capacity and resolution of the assisting chromatography separation(s), hybrid methods have also been developed coupling MRM assays with enrichment of proteins by immunodepletion and size exclusion chromatography or enrichment of peptides by antibody capture (SISCAPA). In essence, the latter approach uses the mass spectrometer as a “second antibody” that has absolute structural specificity. SISCAPA has been shown to extend the sensitivity of a peptide assay by at least two orders of magnitude and with further development appears capable of extending the MRM method to cover the full known dynamic range of plasma (i.e., to the pg/ml level).

In other embodiments, Matrix-Assisted Laser Desorption/Ionization Mass Spectrometry (MALDI-MS) is another method that can be used for studying biomolecules (Hillenkamp et al., Anal. Chem., 1991, 63, 1193A-1203A). This technique ionizes high molecular weight biopolymers with minimal concomitant fragmentation of the sample material. This is typically accomplished via the incorporation of the sample to be analyzed into a matrix that absorbs radiation from an incident UV or IR laser. This energy is then transferred from the matrix to the sample resulting in desorption of the sample into the gas phase with subsequent ionization and minimal fragmentation. One of the advantages of MALDI-MS over ESI-MS is the simplicity of the spectra obtained as MALDI spectra are generally dominated by singly charged species. Typically, the detection of the gaseous ions generated by MALDI techniques, are detected and analyzed by determining the time-of-flight (TO) of these ions. While MALDI-TOF MS is not a high resolution technique, resolution can be improved by making modifications to such systems, by the use of tandem MS techniques, or by the use of other types of analyzers, such as Fourier transform (FT) and quadrupole ion traps.

In situ hybridization (ISH) is used to visualize defined nucleic acid sequences in cellular preparations by hybridization of complementary probe sequences. Through nucleic acid hybridization, the degree of sequence identity can be determined, and specific sequences can be detected and located on a given chromosome. The method comprises of three basic steps: fixation of a specimen on a microscope slide, hybridization of labeled probe to homologous fragments of genomic DNA, and enzymatic detection of the tagged target hybrids. Probe sequences can be labeled with isotopes, nonisotopic hybridization has become increasingly popular, with fluorescent hybridization (Nature Methods 2005, 2, 237-238.) now a common choice as it is considerably faster, usually has greater signal resolution, and provides many options to simultaneously visualize different targets by combining various detection methods.

### Kits

In yet another aspect, the present invention provides kits for aiding a diagnosis of a disease, such as lung cancer, wherein the kits can be used to detect the markers of the present invention. For example, the kits can be used to detect any one or combination of markers described above, which markers are differentially present in samples of patients with disease or a change in health status and normal subjects patients.

In one embodiment, a kit comprises: (a) a substrate comprising an adsorbent thereon, wherein the adsorbent is suitable for binding a marker, and (b) a washing solution or instructions for making a washing solution, wherein the combination of the adsorbent and the washing solution allows detection of the marker as previously described.

Optionally, the kit can further comprise instructions for suitable operational parameters in the form of a label or a separate insert. For example, the kit may have standard instructions informing a consumer/kit user how to wash the probe after a sample of seminal plasma or other tissue sample is contacted on the probe.

In another embodiment, a kit comprises (a) an antibody that specifically binds to a marker; and (b) a detection reagent. Such kits can be prepared from the materials described above.

In either embodiment, the kit may optionally further comprise a standard or control information, and/or a control amount of material, so that the test sample can be compared with the control information standard and/or control amount to determine if the test amount of a marker detected in a sample is a diagnostic amount consistent with a diagnosis of lung cancer.

### Statistics

The statistically meaningful difference may have p values that are statistically meaningfully higher or lower than the expression level of the patient group or control group. Preferably, the p value may be less than 0.05.

Having described the invention with reference to the embodiments and illustrative examples, those in the art may appreciate modifications to the invention as described and illustrated that do not depart from the spirit and scope of the invention as disclosed in the specification. The examples are set forth to aid in understanding the invention but are not intended to, and should not be construed to limit its scope in any way. The examples do not include detailed descriptions of conventional methods. Such methods are well known to those of ordinary skill in the art and are described in numerous publications. All references cited above and in the examples below are hereby incorporated by reference in their entirety, as if fully set forth herein.

### Example 1: Generation of Organ Datasets Using Sequencing-by-Synthesis

Data generated from transcriptomic profiling of 25 human organs was analyzed using sequencing-by synthesis (SBS). Organ-specific proteins as set forth herein resulted in the identification of 2,648 unique organ-specific proteins. As demonstrated by comparing lung-specific proteins with genes that were determined in transcriptomic studies on human diseases, organ-specific panel proteins were highly indicative of diseases or changes of health status.

### SBS Dataset of Human Tissues

The comparative set of biomarkers comprised an analysis of the transcriptomes in specific human organs. Analysis was performed by Solexa (now Illumina, Inc.) San Diego, Calif. A total of 25 human organs were collected from a cohort of healthy donors. Most samples came from donors who died in accidents. Organs were divided and pooled by type and donor gender. Other samples were purchased from vendors.

The data included 64 datasets: some organs contained samples from multiple donors; some samples were analyzed in multiple sequencing runs. A detailed list of the datasets is summarized in Table 6.

Message RNA (mRNA) molecules were extracted from the samples and assessed for quality. Samples of mRNA molecules that passed quality control were sent to Solexa (now Illumina) for transcriptomic analysis under a service contract, using their then existing SBS protocol on the Genome Analyzer [1]. The SBS data set from the analysis of each set of pooled organs contained a list of 20-base tags derived from transcripts in the samples and their corresponding abundance. The tags had a canonical initiation sequence of GATC due to the enzyme used in digesting cDNA molecules. The tags were also annotated under the same annotation system that was used by Solexa (now Illumina) for massive parallel signature sequencing (MPSS) tags [2,3]. The number of SBS tags in individual datasets ranged from 164,918 tags in dataset “HCC59” to 663,447 tags in dataset “HCC20”.

### Analysis of the SBS Data

The SBS data obtained as described above was analyzed to identify organ-specific proteins. First, sequencing errors from tag counts were subtracted and tags whose counts were below sequencing errors were removed. SBS tags are prone to small sequencing errors, particularly in the end portion of the base tags. The following steps were used to estimate and correct sequencing errors occurring in the last bases of tags:
(i) For each dataset, SBS tags that differed in their last bases were grouped together. For example, tags “GATCAAATATCACTCTCCTA” (SEQ ID NO. 1) (count 85974), “GATCAAATATCACTCTCCTC” (SEQ ID NO. 2) (count 673), “GATCAAATATCACTCTCCTT” (SEQ ID NO. 3) (count 173), “GATCAAATATCACTCTCCTG” (SEQ ID NO. 4) (count 39) were grouped together in dataset “HCC01_A”;
(ii) SBS tags that differed in the last bases of the sequence from any primer-dimers were removed from estimating sequencing errors. Primer-dimers used in generating the SBS data were listed in Table 7;
(iii) The most abundant tags were identified from SBS tag groups. In the above example, tag “GATCAAATATCACTCTCCTA” (SEQ ID NO. 1) was identified as the most abundant tag in the group;
(iv) SBS tag groups were removed from estimating sequencing errors if their most abundant tags (1) had counts less than 1,000, (2) were not annotated to classes 1, 2, 3, or 4 under Solexa annotation, or (3) had same counts as any other tags in the same groups. Tag “GATCAAATATCACTCTCCTA” (SEQ ID NO. 1) was annotated as class 4 under Solexa annotation and thus was used for estimating sequencing errors;
(v) Unannotated tags in the remaining SBS tag groups were identified as incidences of sequencing errors, whose rates were estimated by the ratios of counts of unannotated tags to counts of the most abundant tags. In the above example, the most abundant tag was annotated. So an incidence of A->C, A->G, or A->T sequencing error was identified by each of the three unannotated tags. The corresponding error rate was estimated at 673/85,974=0.0078, 39/85,974=0.00045, or 173/85,974=0.0020, respectively;
(vi) Sequencing error rates in each dataset were estimated by the medians of corresponding incident sequencing error rates in the dataset;
(vii) The overall sequencing error rates were estimated by the medians of corresponding sequencing error rates in individual datasets and were listed in Table 8;
(viii) For each SBS dataset, contributions by sequencing errors of the most abundant tags to counts of other tags in the same SBS tag groups were estimated by multiplying the counts of the most abundant tags with the corresponding sequencing error rates listed in Table 8. Sequence errors were rounded up to integers and subtracted from the counts of other tags; and
(ix) Only SBS tags with positive tag counts after correcting for sequencing errors were kept for further analysis.

Second, sequences of primer-dimers and sequences of REPEAT were removed. SBS tags that are ubiquitous in human genome were annotated as REPEAT under Solexa annotation. These tags were not reliable for measuring transcripts in samples and were thus removed from further analysis. Similarly, SBS tags that were identical to primer-dimers listed in Table 7 were also removed from further analysis.

Third, SBS tags to RNA RefSeq sequences were annotated and unannotated tags were removed. Two files of RNA RefSeq sequences were downloaded from National Center for Biotechnology Information (NCBI) website: (1) “human.rna.fna.gz” (43,504 sequences, from ftp.ncbi.nih.gov/refseq/H_sapiens/mRNA_Prot/); and (2) “rna.fa.gz” (42,753 sequences, from ftp.ncbi.nih.gov/refseq/H_sapiens/H_sapiens/RNA/). Sequences in the two files were combined and reconciled, which led to a list of 44,706 RNA RefSeq sequences. The sequences were then theoretically digested into 20-base tags with an initiation sequence of GATC. Both sense and antisense tags were kept. Unique tags were then annotated to RNA RefSeq accession numbers: (1) if they belonged to any sense sequences of RNAs, they were classified as “F” (for “forward”) and annotated with the corresponding RefSeq accession numbers; (2) if they belonged to antisense sequences of RNAs, they were classified as “B” (for “backward”) and annotated with the corresponding RefSeq accession numbers. It was common for a single SBS tag to be annotated to multiple RNAs. For example, tag “GATCAAAAAAACGTTCTTTG” (SEQ ID NO. 5) was classified as “F” and annotated to RNAs “NM_001025091.1” and “NM_001090.2”; and tag “GATCAAAAAAAAATTTTTGC” (SEQ ID NO. 6) was classified as “B” and annotated to RNAs “NM_001136275.1” and “NM_024595.2”. A total of 176,384 tags were classified as “F” and 168,605 as “B”. SBS tags that could not be annotated to RefSeq accession numbers were removed from further analysis.

Fourth, data was normalized to transcript per million (TPM) and all SBS data was assembled into a single file. Individual datasets were normalized by TPM, the same method used for normalizing MPSS data [2,3]. Briefly, a global normalization factor was calculated for each dataset by dividing a million by the total count of all remaining SBS tags in the dataset. Individual tag counts were then multiplied by the normalization factor and rounded up to integers. Only SBS tags with positive tag counts were kept for further analysis. The number of remaining SBS tags in individual datasets ranged from 27,864 tags in dataset “HCCHuHep” to 68,933 tags in dataset “HCC29”. All remaining SBS data were assembled into a single data file as a tag vs. dataset array. There were 192,647 unique SBS tags in the file. This file was used for downstream analysis.

Fifth, SBS tags having normalized counts that were below a cutoff of 10 were removed from all samples. To estimate the noise level in SBS data, replicate datasets generated from same samples were compared. For each pair of replicate datasets, coefficients of variation (CVs) and maximum counts from counts of individual tags were calculated first. Tags with same maximum counts were then grouped together and the corresponding median CVs were calculated. In the case where there were less than 100 tags in a group, tags with lower and higher maximum counts were added to the group until 100 or more tags were included. In the case where 100 or more tags were included, the maximum count of the group was replaced by the corresponding median.

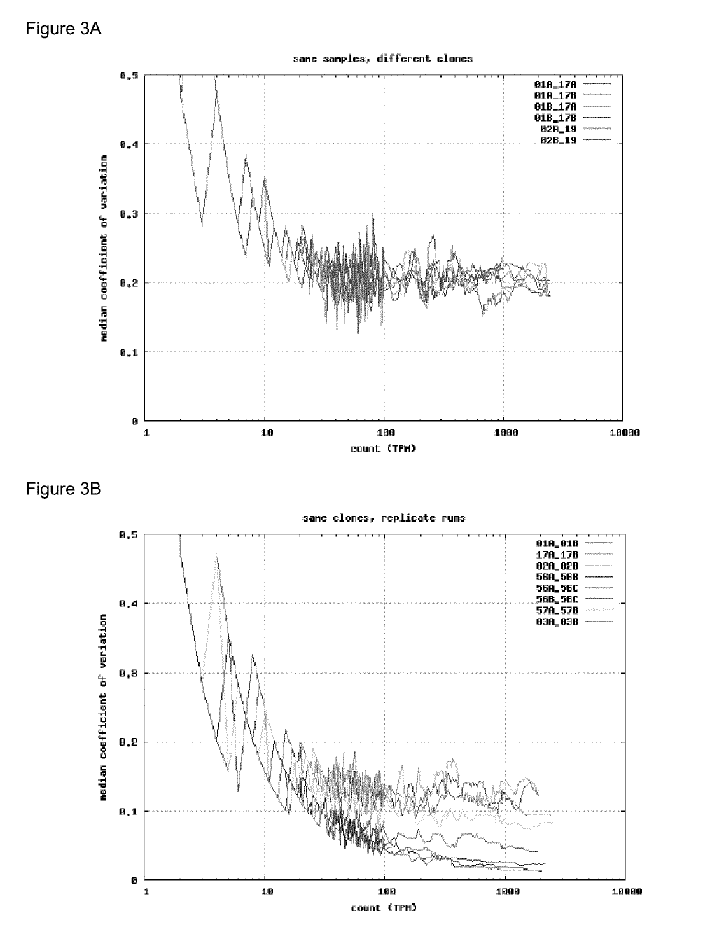

Two types of replicate datasets resulted: (1) datasets generated from different cDNA clones of same mRNA samples and (2) datasets generated in different sequencing runs on same cDNA clones. FIG. 3A and FIG. 3B illustrate the median CV vs. maximum tag count for both types of replicate datasets. Median CVs remained relatively flat for most values of tag count; however, a dramatic increase is shown as the tag count approached 10, indicating SBS data were no longer reliable at that level. A cutoff of 10 was thereby selected as the noise level in SBS data. SBS tags having normalized counts that were below the cutoff in all samples were removed from further analysis. A total of 32,853 SBS tags were kept.

Sixth, removed SBS tags that could not be mapped to proteins were removed. Some SBS tags were annotated to non-coding RNAs. Such tags were not useful for identifying organ-specific proteins and needed to be removed from further analysis. The following steps were carried out to determine which SBS tags to remove in accordance with this step:
(i) Two files of protein RefSeq sequences were downloaded from NCBI website: (1) “human.protein.faa.gz” (37843 sequences, from ftp.ncbi.nih.gov/refseq/H_sapiens/mRNA_Prot/); and (2) “protein.fa.gz” (37391 sequences, from ftp.ncbi.nih.gov/refseq/H_sapiens/H_sapiens/protein/). Sequences in the two files were combined and reconciled, which resulted in a list of 38,410 protein RefSeq sequences;
(ii) Two files (“gene2accession.gz” and “gene2refseq.gz”) were downloaded from NCBI website (ftp.ncbi.nih.gov/gene/DATA/). The files contained the mappings between Entrez genes, protein RefSeq accession numbers and RNA RefSeq accession numbers. Information in the files were parsed and reconciled along with information in the combined protein RefSeq sequence file. A total of 38,385 protein Refseq accession numbers were assembled along with corresponding genes and RNA RefSeq accession numbers;
(iii) SBS tags were mapped to protein RefSeq accession numbers via their annotation to RNA RefSeq accession numbers and the mapping between protein and RNA RefSeq accession numbers;
(iv) SBS tags that could not be mapped to proteins were removed from further analysis. A total of 31,867 SBS tags were kept.

Seventh, the SBS tag counts were condensed to protein abundance. It was common that multiple SBS tags were mapped to same proteins. To determine the abundance of proteins in our samples, the following steps were carried out to condense the SBS tag counts to protein abundance:
(i) For each protein, all SBS tags mapped to the protein were collected;
(ii) The most abundant SBS tag (as evaluated by the total tag count in all datasets) was identified for the protein;
(iii) Less abundant SBS tags of the protein were removed from further analysis if their abundance satisfied any of these three conditions: (1) their total tag count in all datasets was less than half of that of the most abundant tag, (2) their highest count in all datasets was less than 50, or (3) their Pearson correlation with the most abundant tag was greater than 0.5. The majority of proteins kept their most abundant SBS tags after this step. A few proteins however kept two comparable but uncorrelated SBS tags, likely due to alternative splicing in the corresponding mRNAs;
(iv) SBS tags were also removed from further analysis if they (1) could be mapped to another protein and (2) would be removed from that protein under conditions listed above;
(v) Some SBS tags could be mapped to proteins of multiple genes. In such cases, predicted proteins were removed from the list of proteins that were mapped to the tags. SBS tags that were mapped to predicted proteins of multiple genes were removed from further analysis;
(vi) A total of 15,267 SBS tags were kept. Their tag counts were used for measuring protein abundance in the samples.

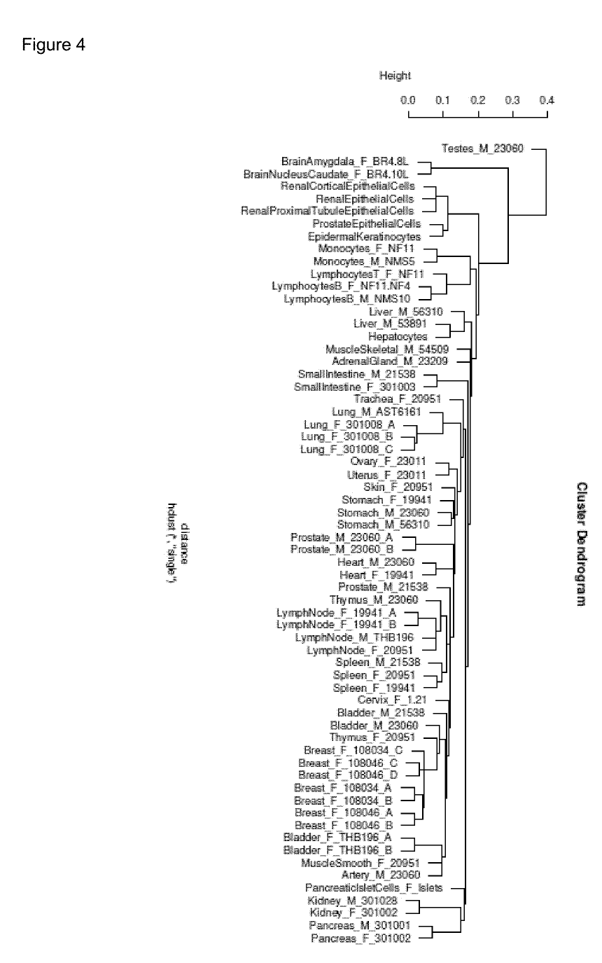

Eighth, the quality of the SBS data was assessed, and outlier datasets were removed. To assess the quality of SBS data in profiling human organs, unsupervised clustering was carried out on the data. The distance between two datasets was evaluated as 1−ρ, where ρ was the Spearman's rank correlation coefficient. The clustering was carried out on R function “hclust” using a “single” method (see www.r-project.org/). The result was plotted in FIG. 4. Most datasets of same organs were clustered together or nearby. The exceptions were two datasets of muscle, two datasets of thymus and five datasets of epithelial cells, which were clustered together regardless of their organ origins. The five datasets of epithelial cells and the two datasets of hepatocytes and of pancreatic islet cells were removed from further analysis.

Ninth, the different datasets were condensed into data of different organs. As listed in Table 6, some organs included multiple samples and some samples generated multiple datasets. To compare protein abundance in different organs, the SBS data of different datasets were condensed into SBS data of different organs according to the following steps:
(i) Quantile-quantile (QQ) normalization [4] was applied to datasets of same samples to reduce technical variations in the datasets. Protein abundance in the samples was then estimated by the corresponding median in their belonging datasets;
(ii) QQ normalization was also applied to SBS data of samples of same organs to reduce biological variations in the samples. Protein abundance in the organs was then estimated by the corresponding median in their belonging samples;
(iii) SBS tags whose counts were less than 10 in all 25 organs were removed from further analysis;
(iv) The remaining 14,561 SBS tags were assembled in a tag vs. organ array and stored in a single file.

### Example 2: Identification and Relevance of Organ-Specific Proteins

To evaluate whether a protein was organ specific, its abundance in different organs was sorted from high abundance to low abundance. More specifically, we sorted the SBS tag counts of the protein were sorted so that n₁≥n₂≥ . . . ≥n₂₅, wherein nᵢ was the tag count in organ i. The protein was specific to the first k organs if its tag counts satisfied all three conditions listed below:
(i) Tag counts in the first k organs were at or above the noise level of SBS data while those in other organs were below the noise level, i.e., nₖ≥10 and nₖ₊₁<10;
(ii) Tag counts in the first k organs were significantly above those in other organs. This condition was determined by application of an exact binomial test to calculate the p value of distinguishing the drawing of nₖ tags from a total of S₂₅ tags with the drawing of nₖ₊₁ tags from S₂₅ tags, where S₂₅ was the total tag count in all organs. The difference was considered significant if the two-sided p value was no greater than 0.05; and
(iii) The total tag count in the first k organs was at least half of the total in all organs, i.e., Sₖ/S₂₅≥0.5, where Sₖ was the total tag count in the first k organs.

Proteins were identified that were specific to up to five organs, i.e., k≤5. Proteins specific to different organs were summarized in Table 5. Proteins of different RefSeq accession numbers but of same genes were grouped together and counted as single proteins. Proteins specific to more than one organ were summarized by number of proteins that correspond to each organ. As indicated in Table 5, a total of 2,648 unique proteins were identified as organ specific and were attributed to 4,239 entries.

### Example 3: Identification of Lung-Specific Panel Proteins, Lung-Specific Panels, and Relevance to Diagnosis of Lung-Related Diseases

To demonstrate the relevance of the organ-specific proteins identified above to diseases of corresponding organs, 115 lung-specific proteins (k≤5) identified in Table 5 (**) were compared with genes that were identified in transcriptomic studies described above for many major human diseases. Lung-specific proteins were uploaded to the NextBio database (www.nextbio.com). The NextBio database is a collection of results from most publicly available transcriptomic studies. We reviewed a total of 1,421 studies on human diseases and selected those studies that indicated at least one lung-specific protein for the diseases. The studies were sorted from high to low by their correlation with lung-specific proteins. The top 50 studies were listed in Table 9.

Comparison Between Lung-Specific Proteins and Disease-Relevant Genes.

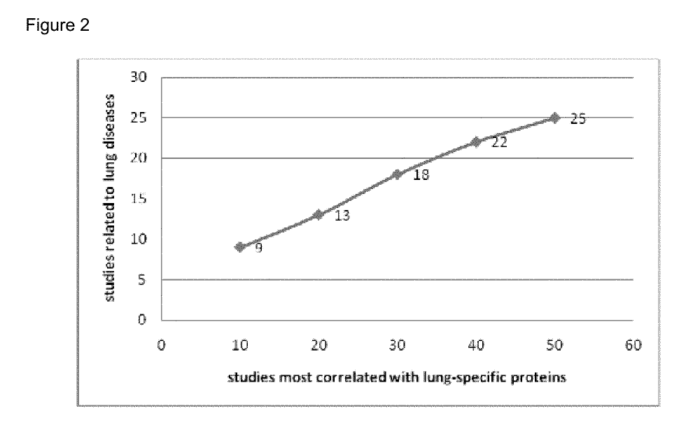

The results of the comparison of the 115 lung-specific proteins to the genes indicated in the transcriptomic studies identified by NextBio are illustrated in FIG. 2: Nine out of the top ten studies and 25 out of the top 50 studies were related to lung diseases including lung cancers. This example clearly demonstrates that organ-specific proteins are highly indicative of diseases of the corresponding organ.

To identify individual proteins that are indicative of lung diseases, we re-analyzed the data related to 115 lung-specific proteins and compared with the proteins that appeared in the top 26 studies on lung diseases. The results are summarized in Tables 1 and 2.

Potential Biomarkers for Lung Diseases or Lung Cancers.

Further, the top 10 studies on lung diseases (including lung cancers) and the top 10 studies exclusively on lung cancers were identified and the lung-specific proteins that were indicated in the studies were collected. The two sets of lung-specific proteins were listed in Table 3 and Table 4, respectively. The proteins were sorted from high to low first by their total occurrence in the corresponding studies and then by their total weight in the studies. Since a study may contain multiple datasets and a protein may be indicated in some datasets, each protein in each study was weighed by the fraction of datasets in which the protein was indicated. For the top 10 studies on lung diseases, SLC39A8 occurred in all studies, 12 proteins (NKX2-1, SFTPB, C4BPA, SFTPD, FAM65B, SFTPA2B, CEACAM6, CTSE, FOXA2, TREM1, LRRC36, and ETVS) occurred 9 times, and 73 proteins occurred at least 5 times. For the top 10 studies on lung cancers, 5 proteins (SFTPB, CLDN18, SFTPD, CPB2 and CEACAM6) occurred in all studies, 9 proteins (SLC39A8, WIF1, NKX2-1, PPBP, ALOX15B, CTSE, SFTPC, FOXA2, and ETVS) occurred 9 times, and 69 proteins occurred at least 5 times. These proteins have a high potential to be biomarkers for the corresponding diseases.

Definition of Organ-Specific Panels.

As described in Example 1, organ-specific panel proteins are specific to multiple organs. A panel of n proteins is specific to an organ if the following two conditions are satisfied:
(i) The n proteins are specific to the organ under the extended definition of organ-specific proteins, as described herein; and
(ii) The joint specificity of the panel in the organ is no less than 0.5. More specifically, assume the specificities of the p=1, n proteins in the o=1, . . . , M organs are {sₙₒ} with sₚ₁+sₚ₂++s_{pM}=1 for all p. The joint specificity of the panel in an organ is then defined as sₒ=c*s₁ₒ*s₂ₒ* . . . *sₙₒ where c is a constant sₒ that s₁+s₂+ . . . +s_{M}=1. The panel is specific to an organ if the corresponding sₒ≥0.5. Clearly a panel can be specific to a single organ.

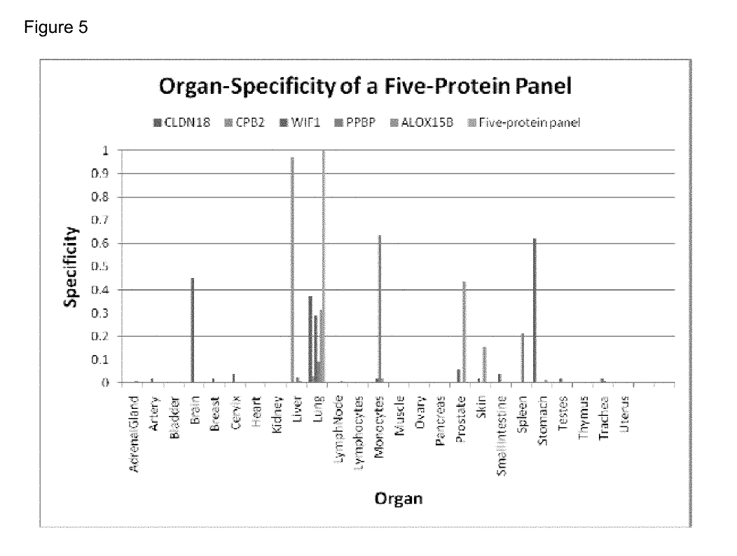

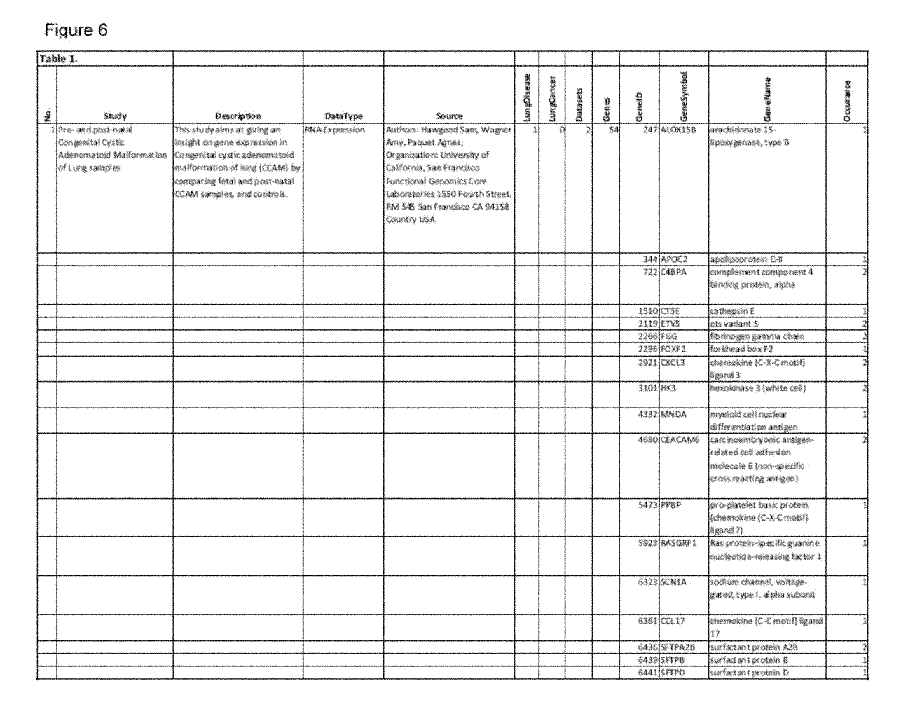

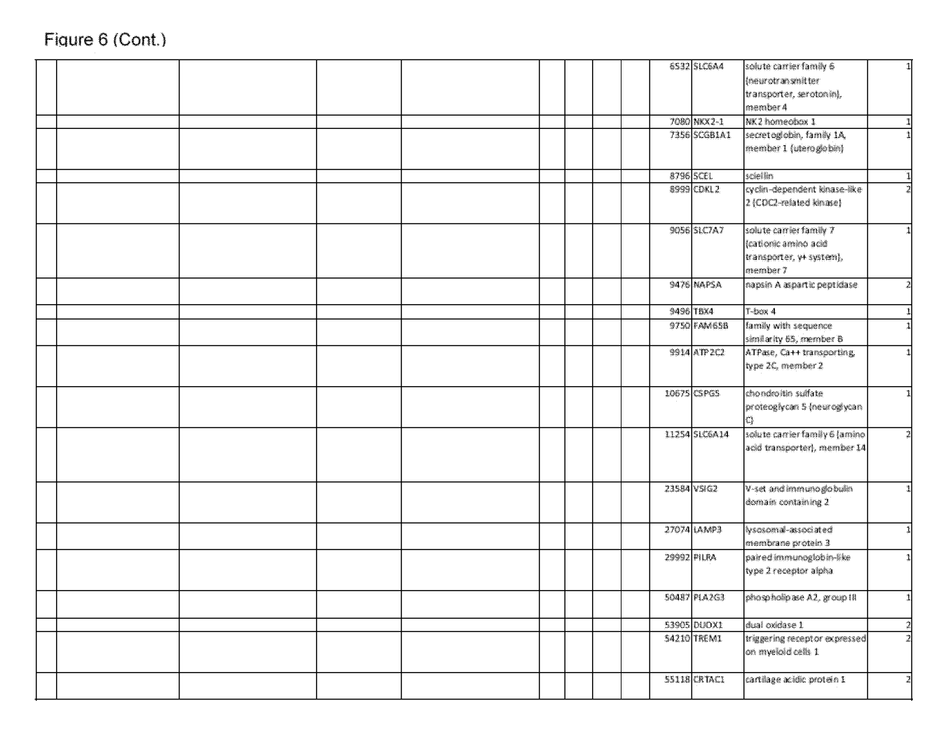

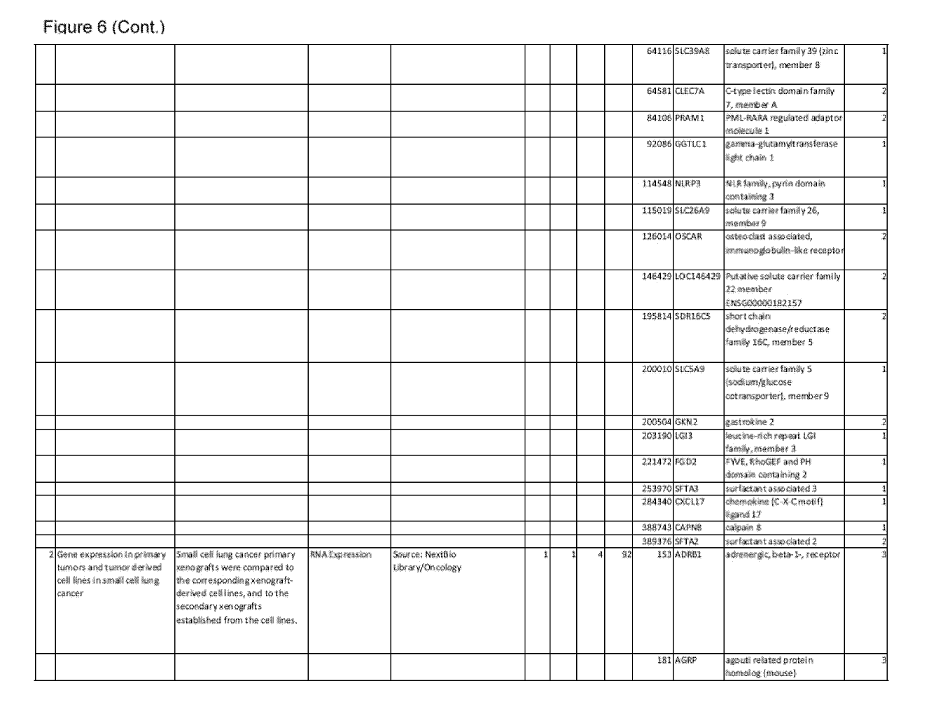

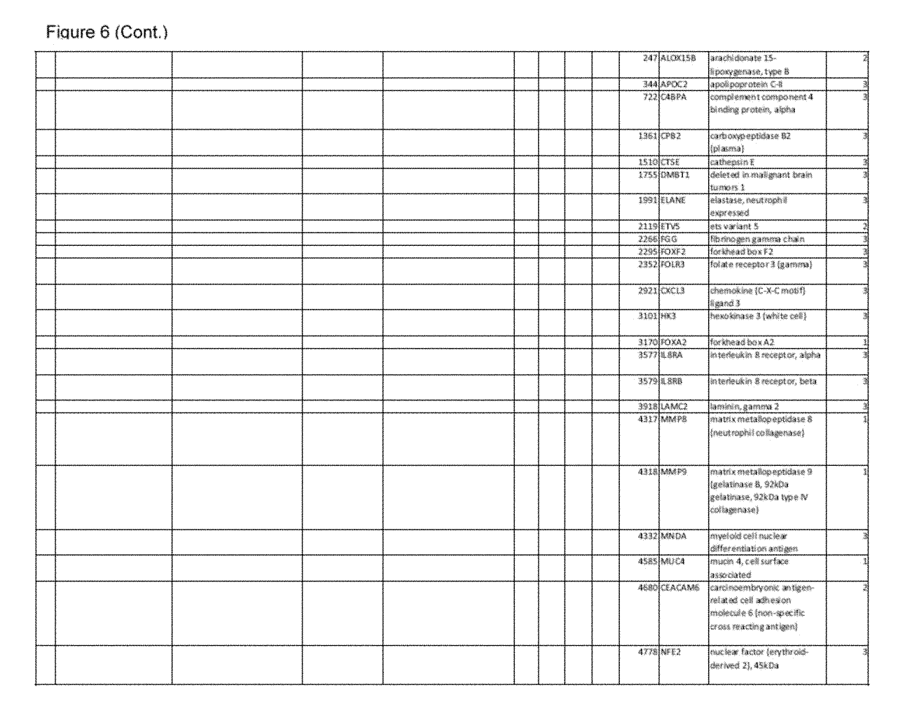

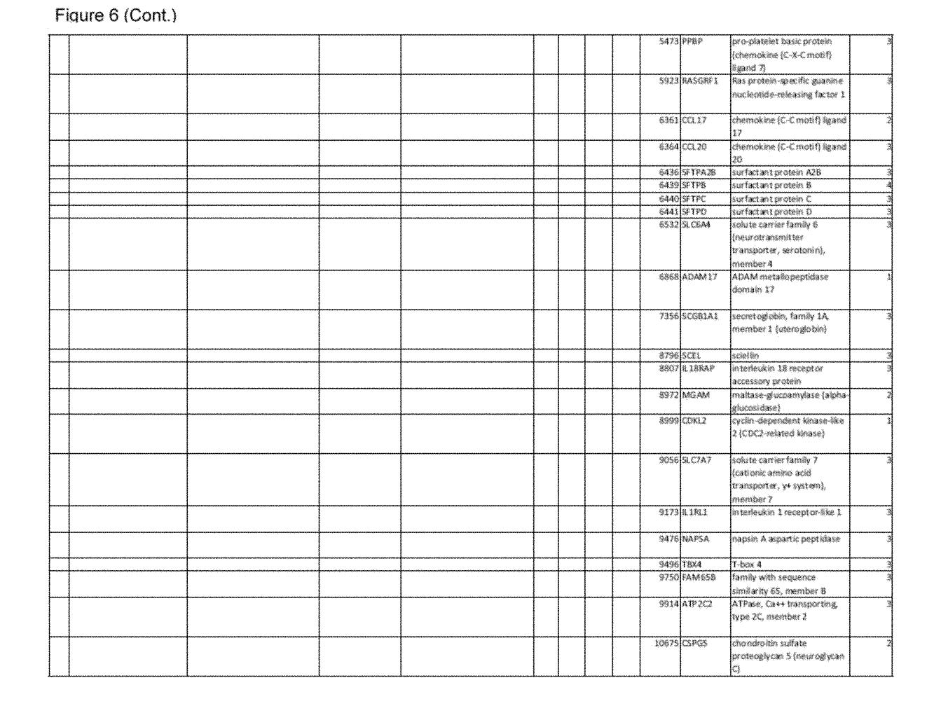

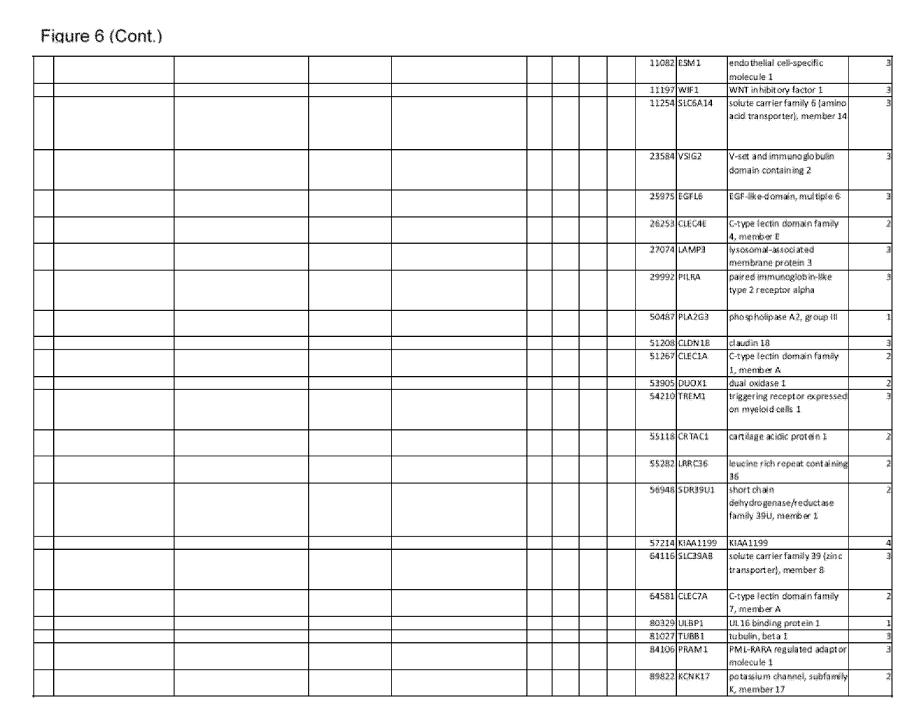

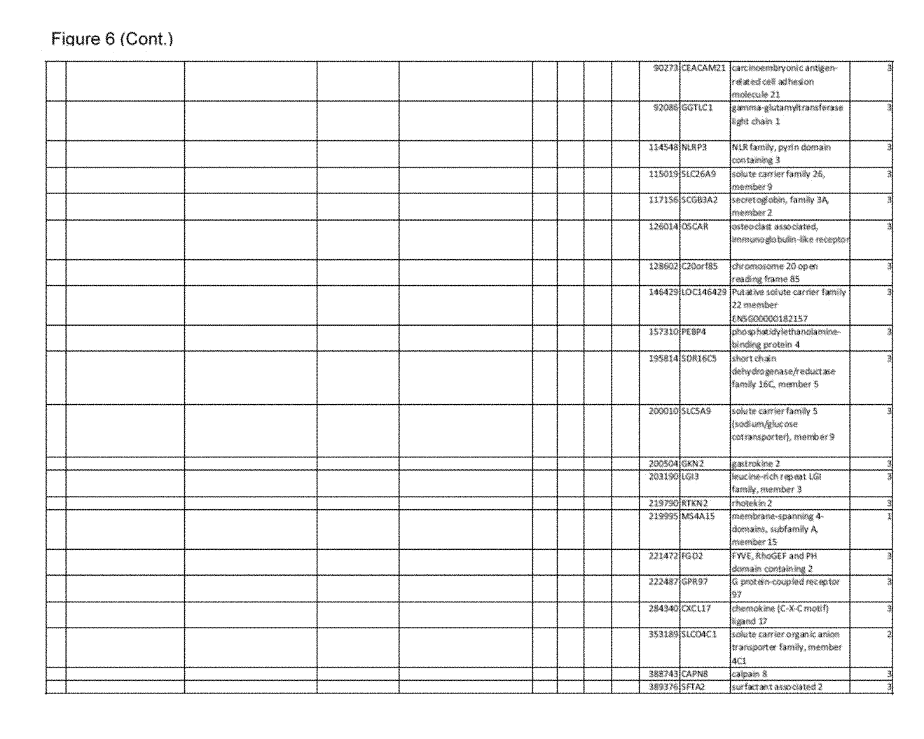

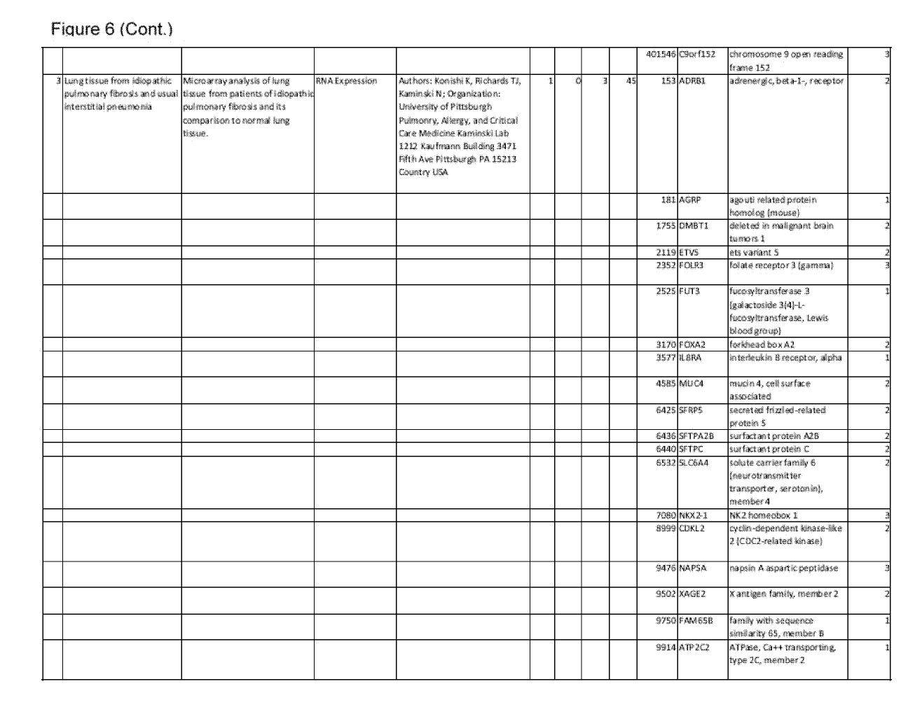

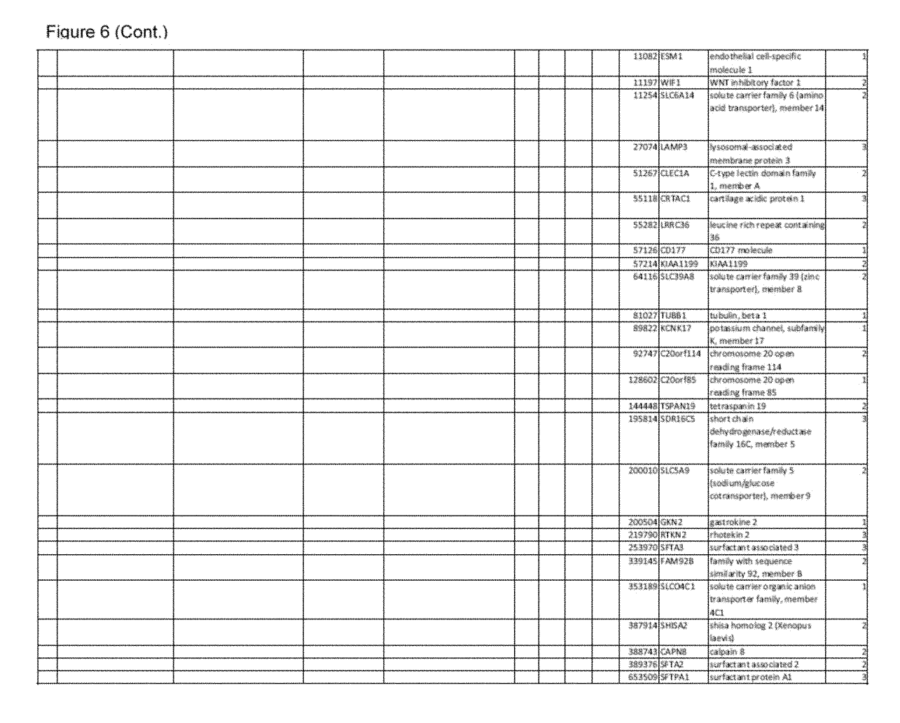

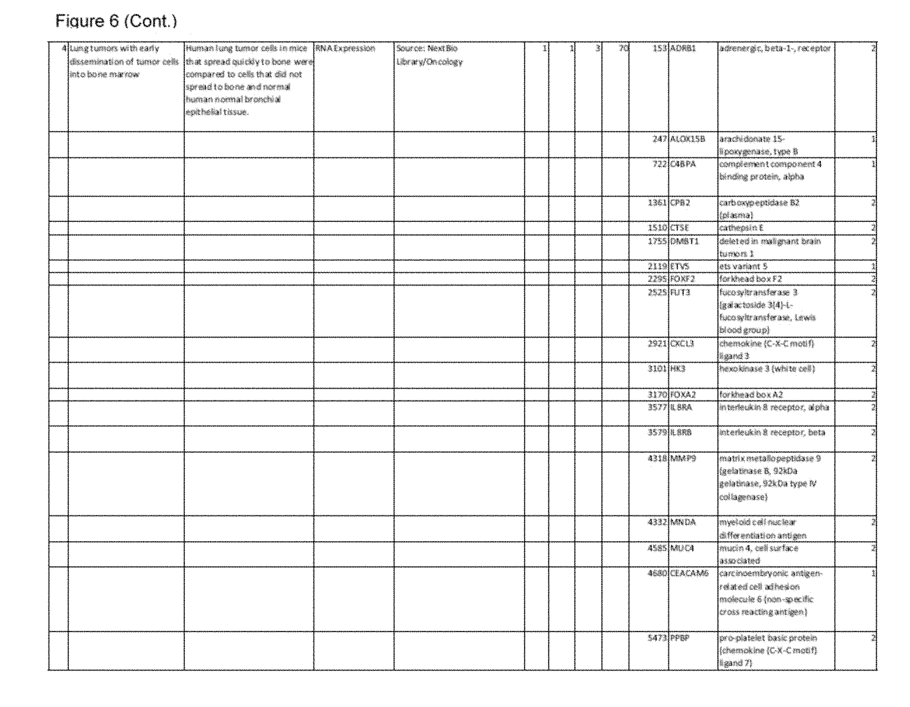

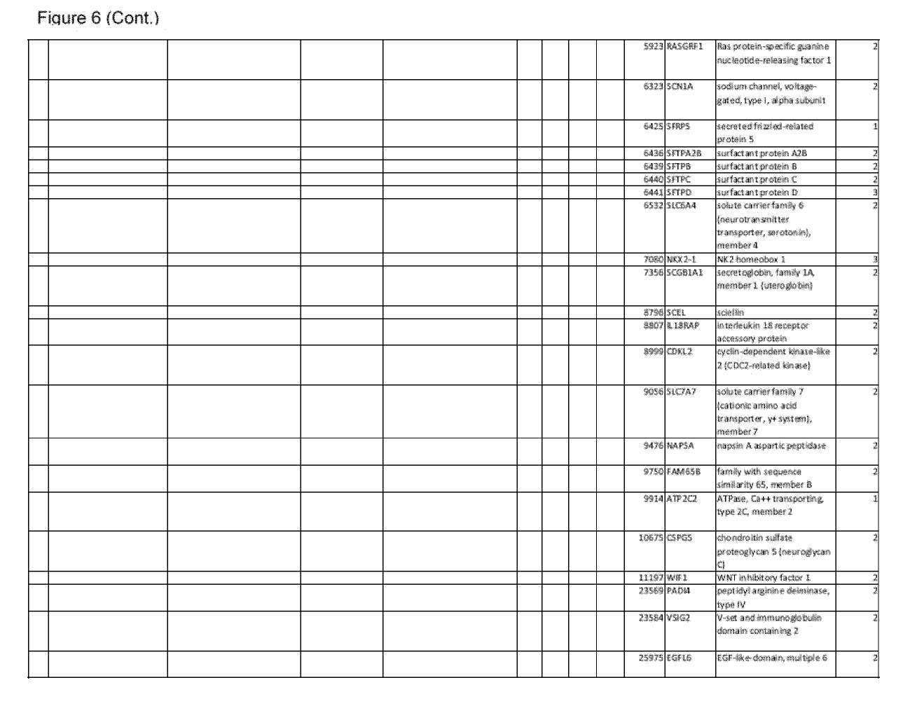

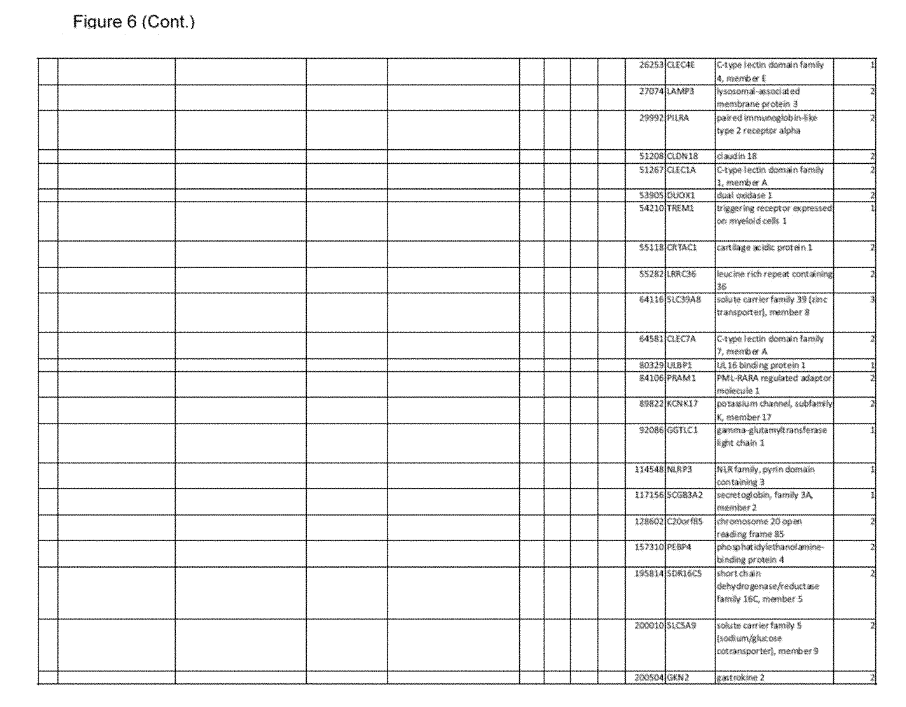

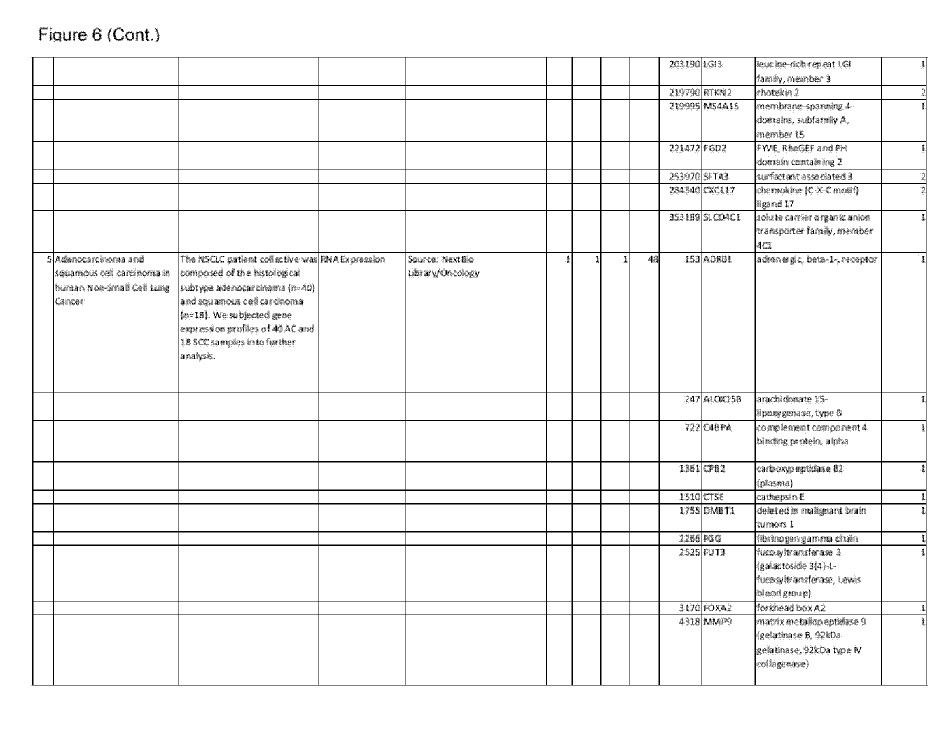

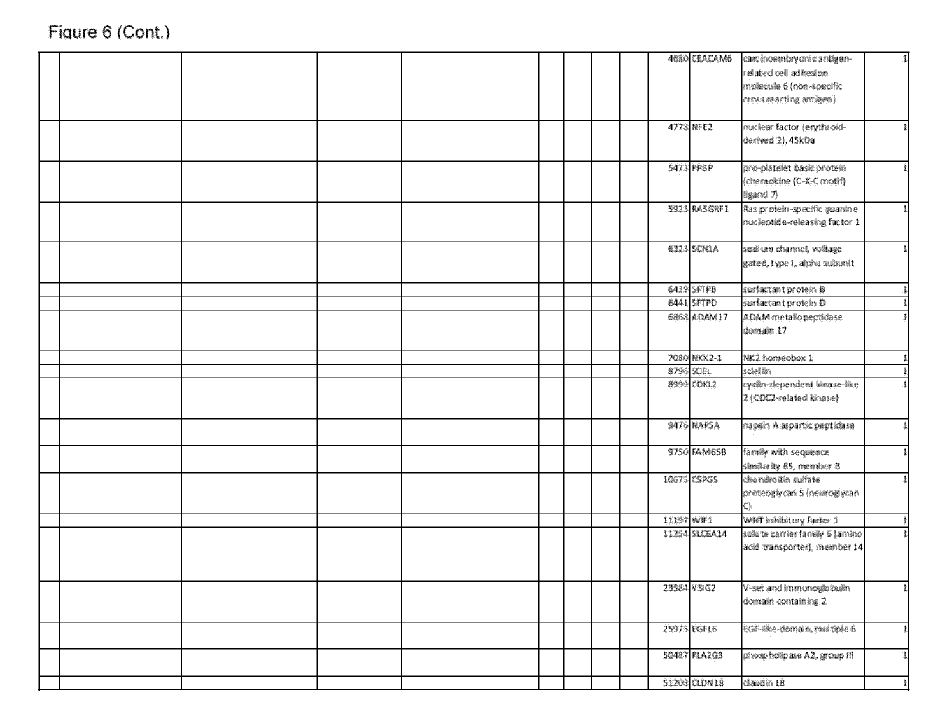

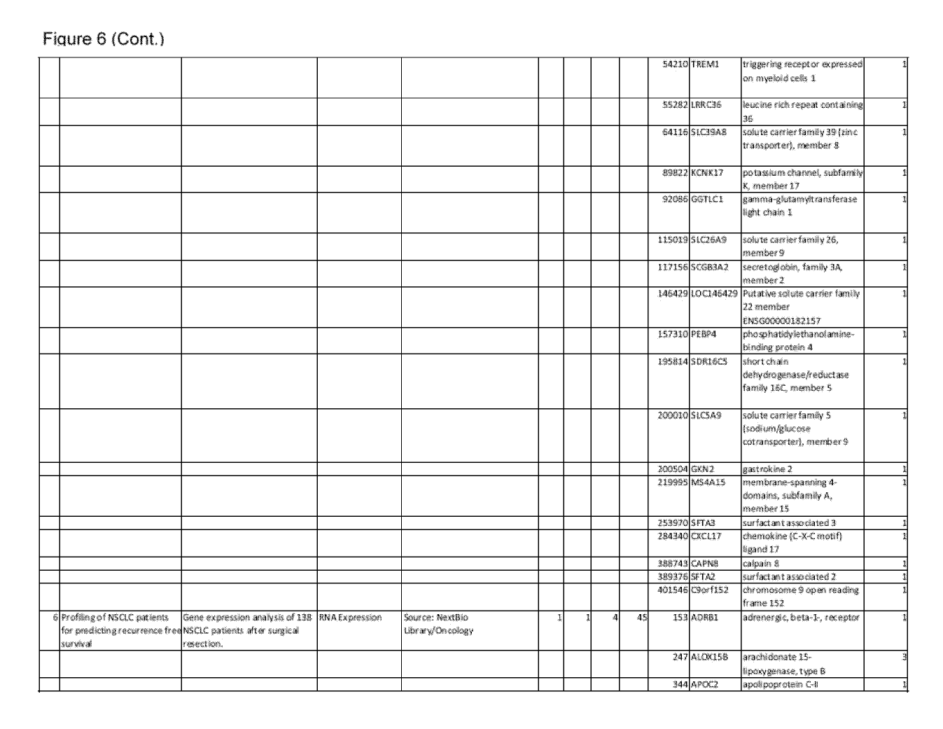

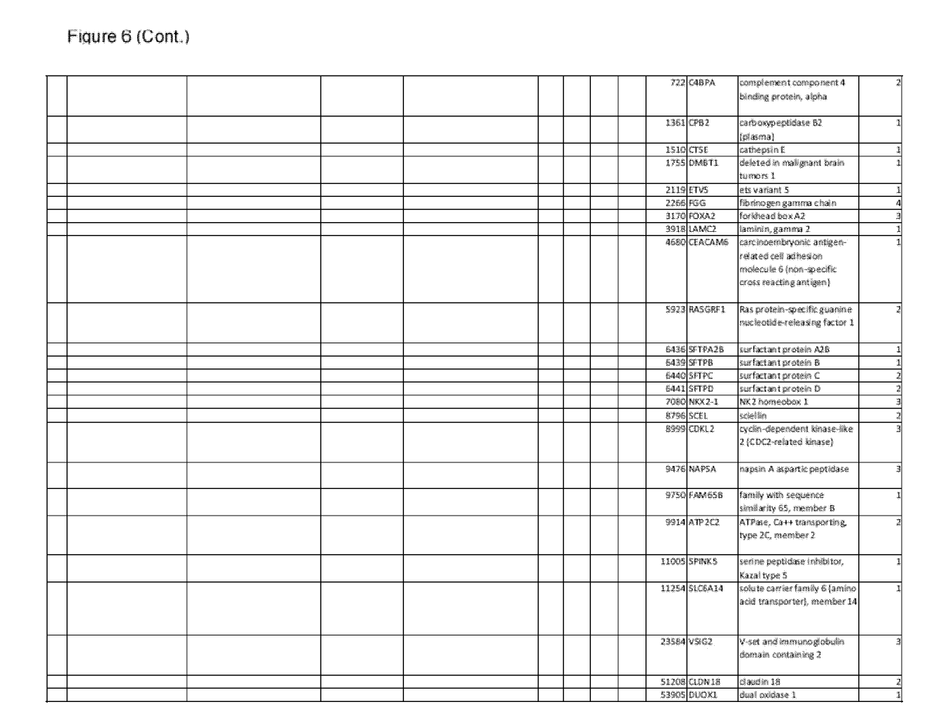

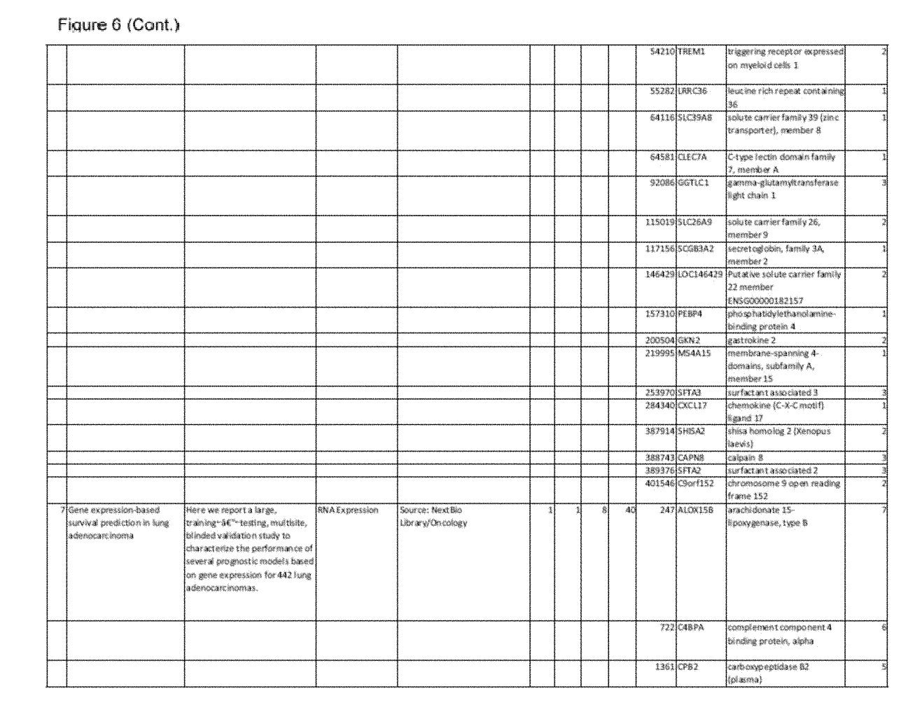

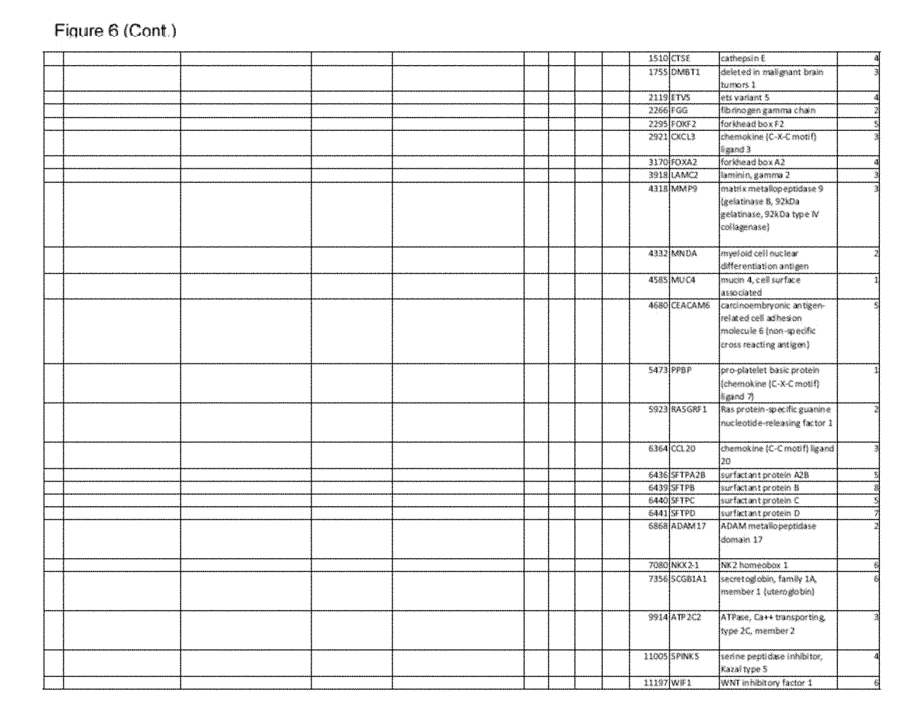

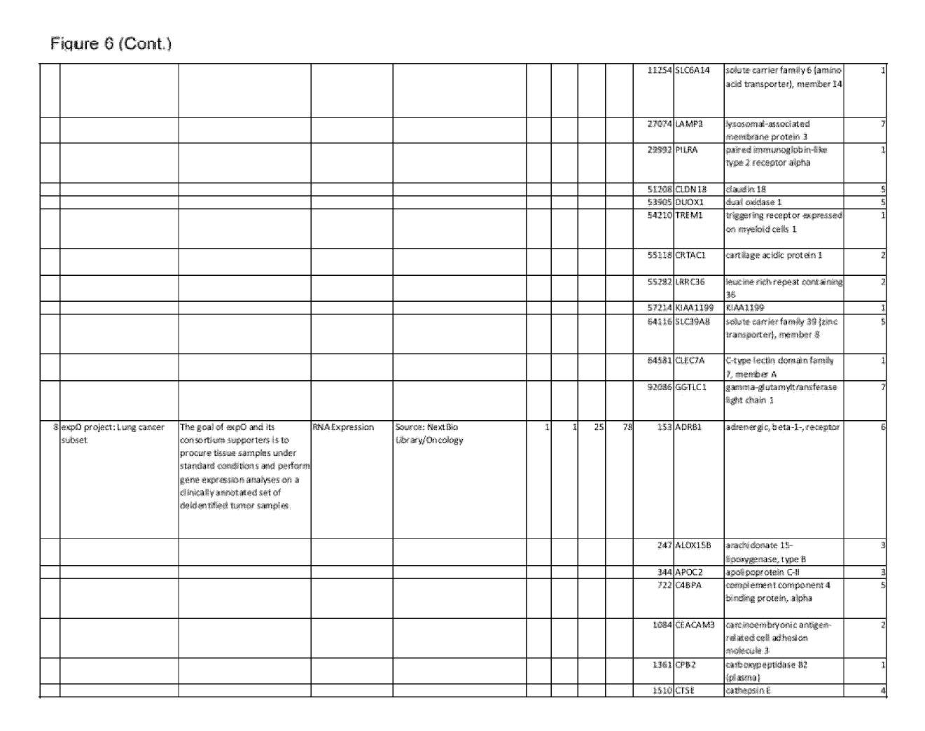

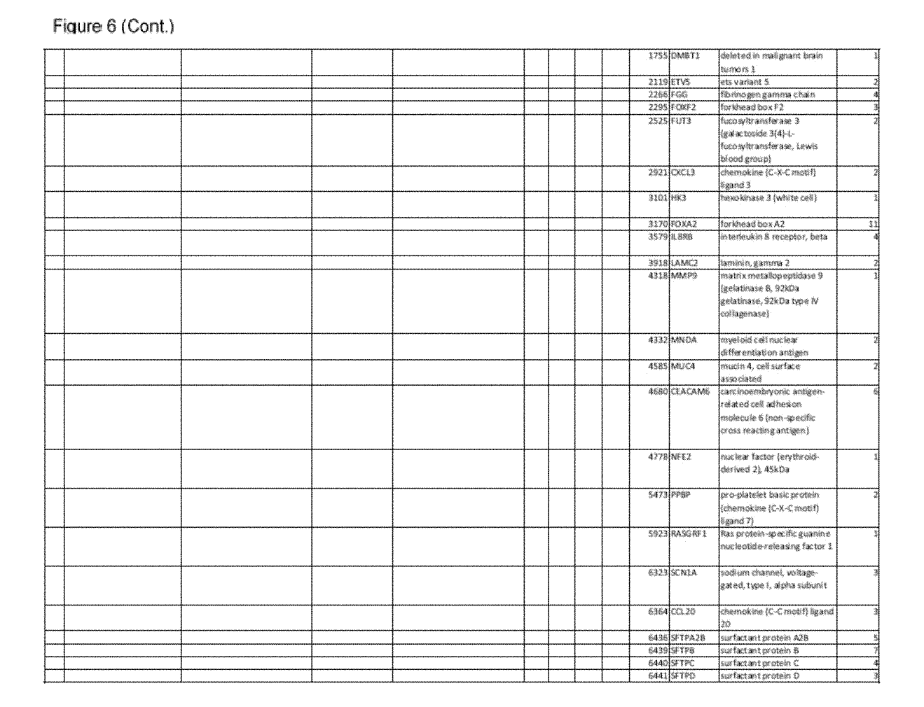

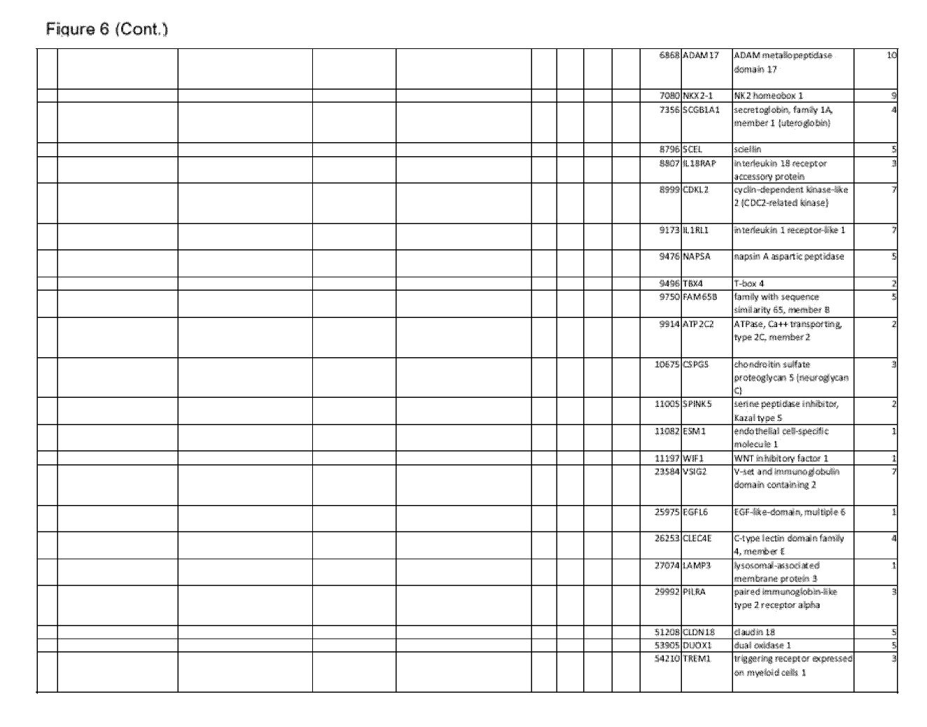

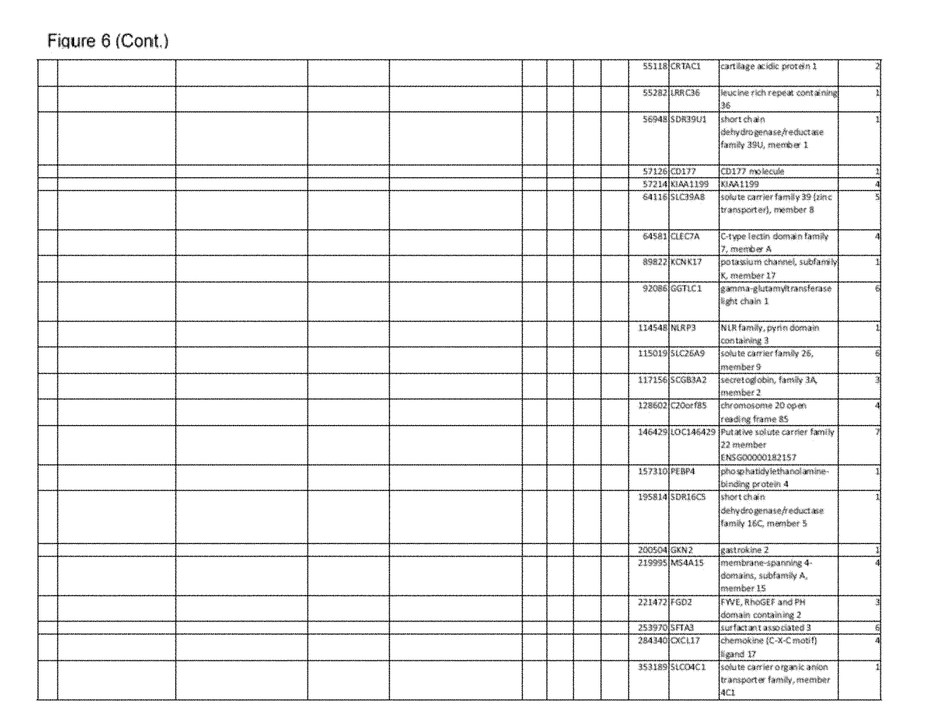

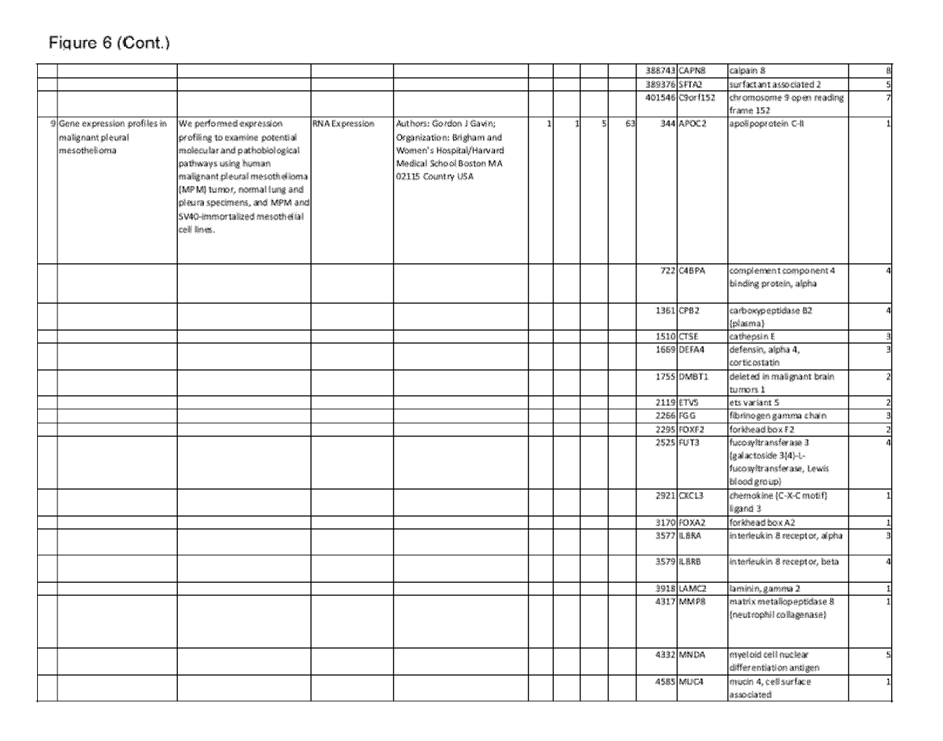

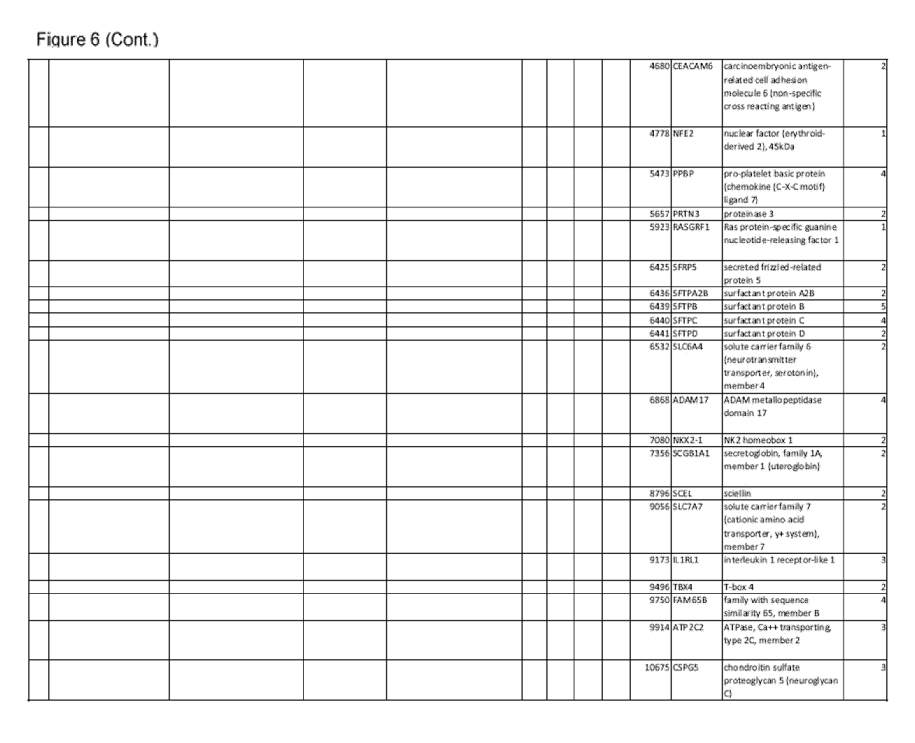

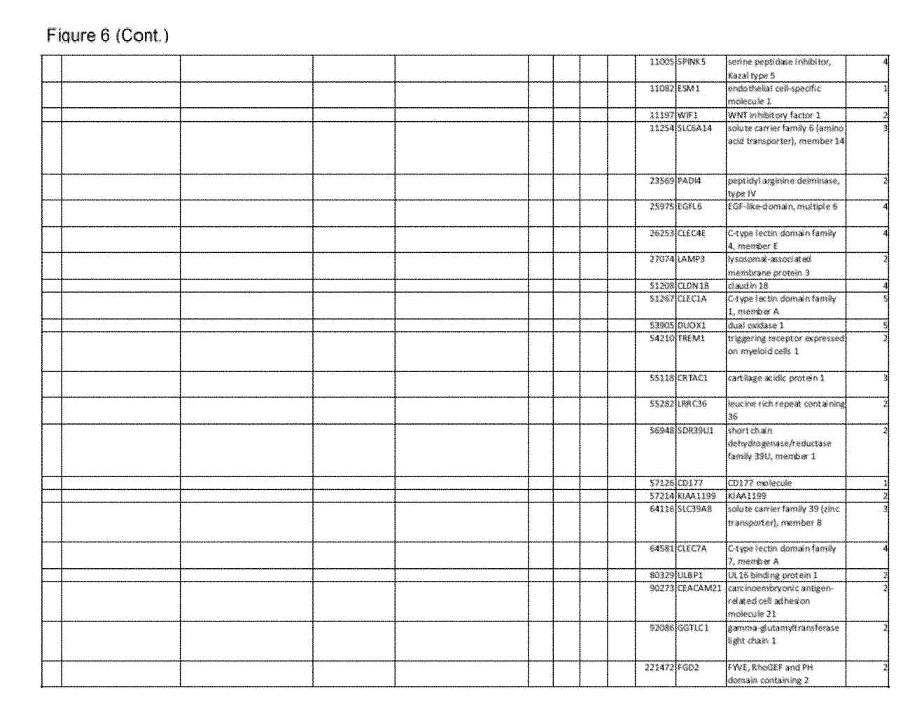

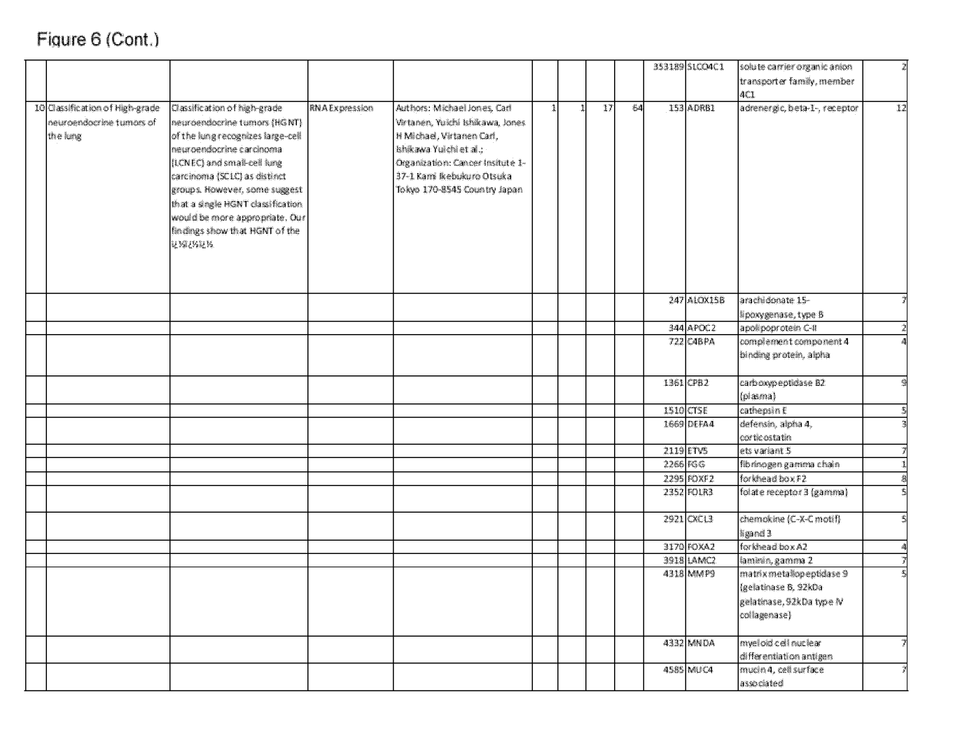

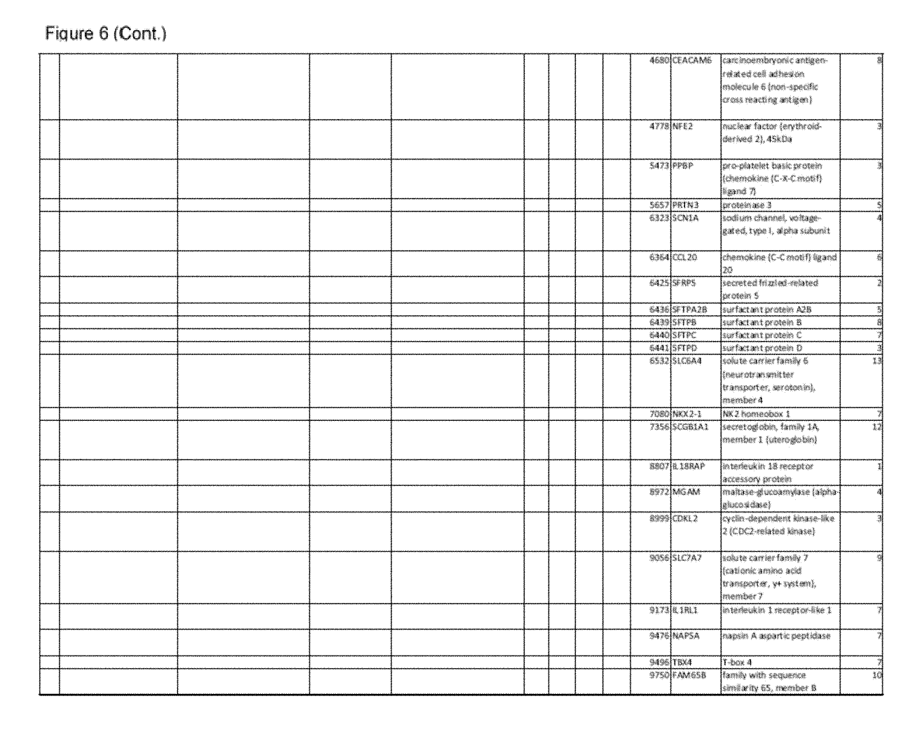

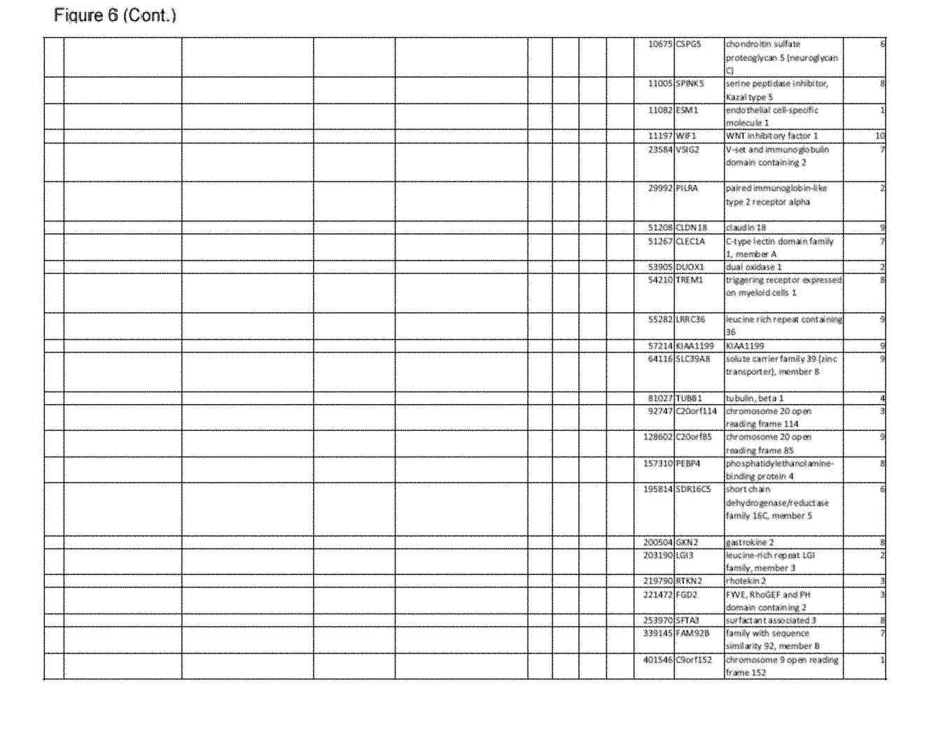

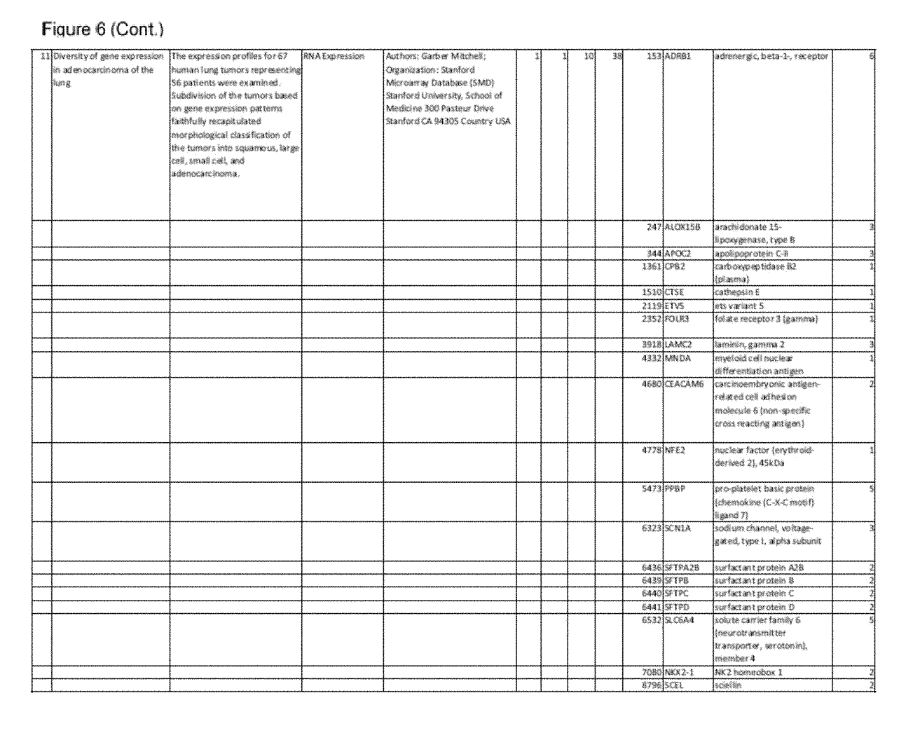

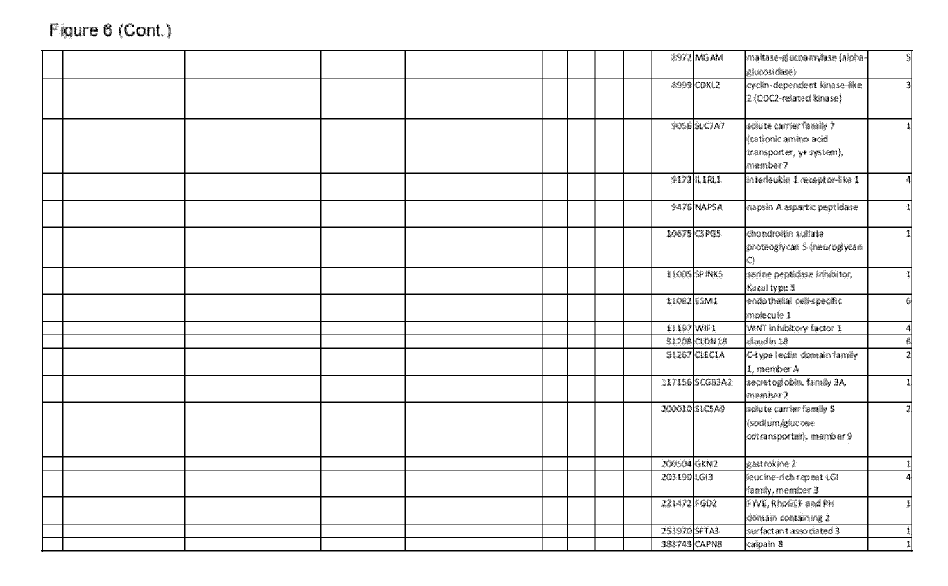

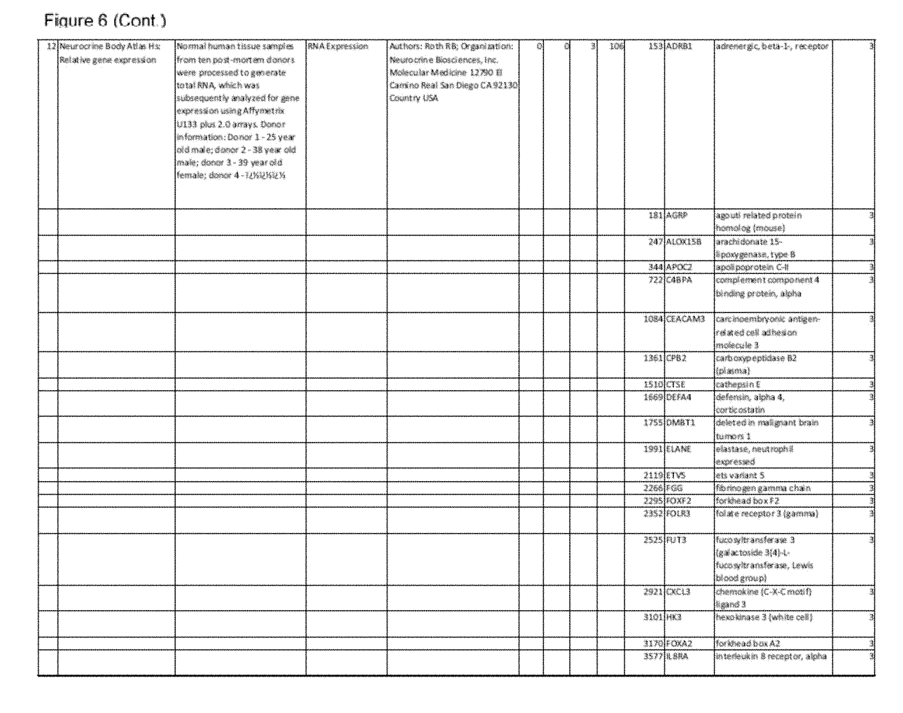

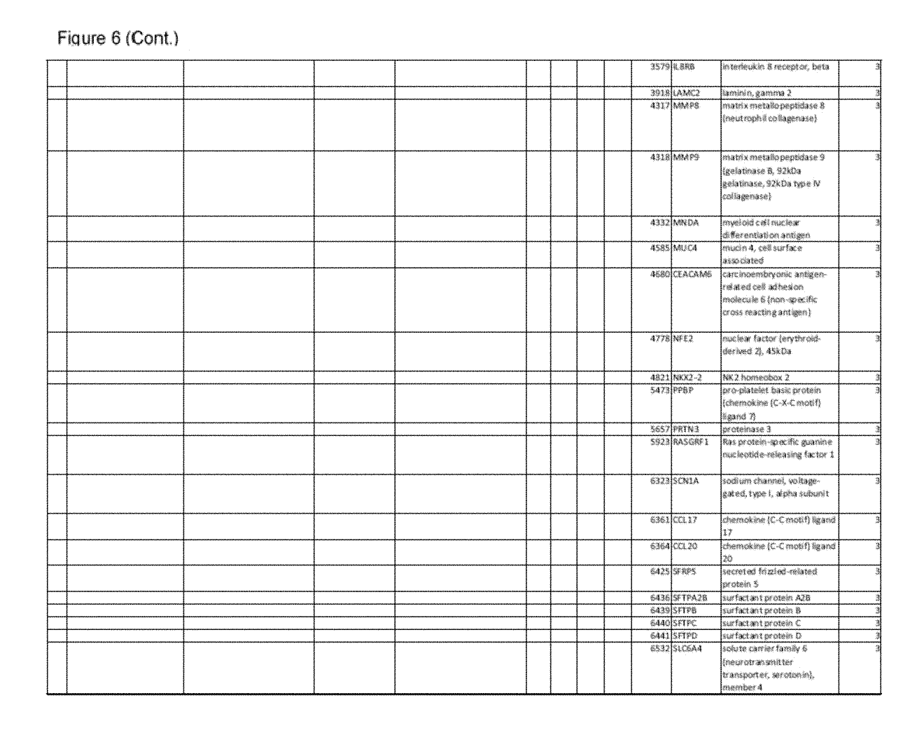

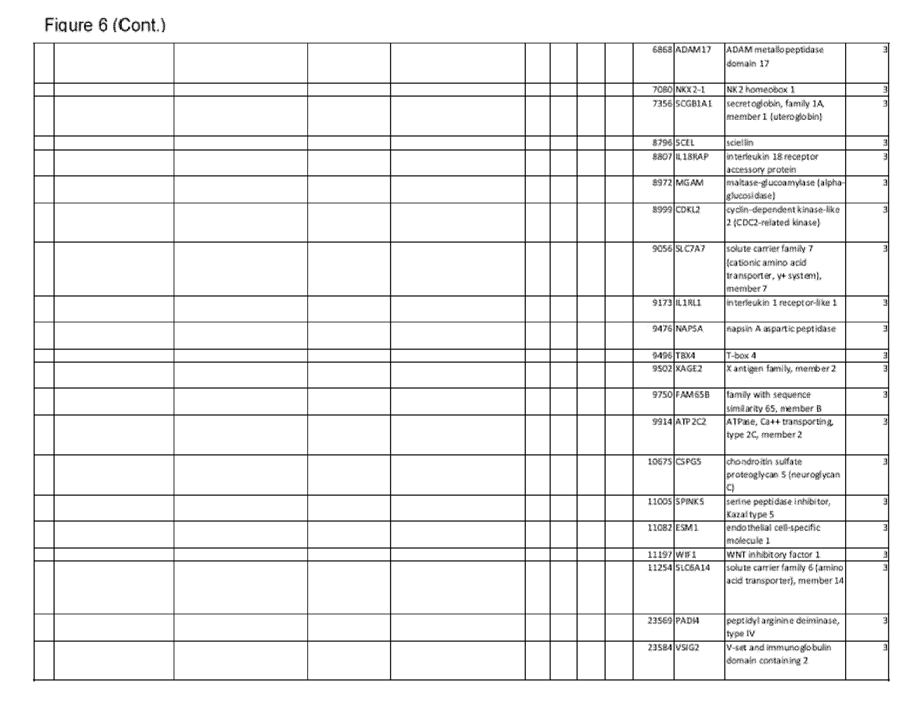

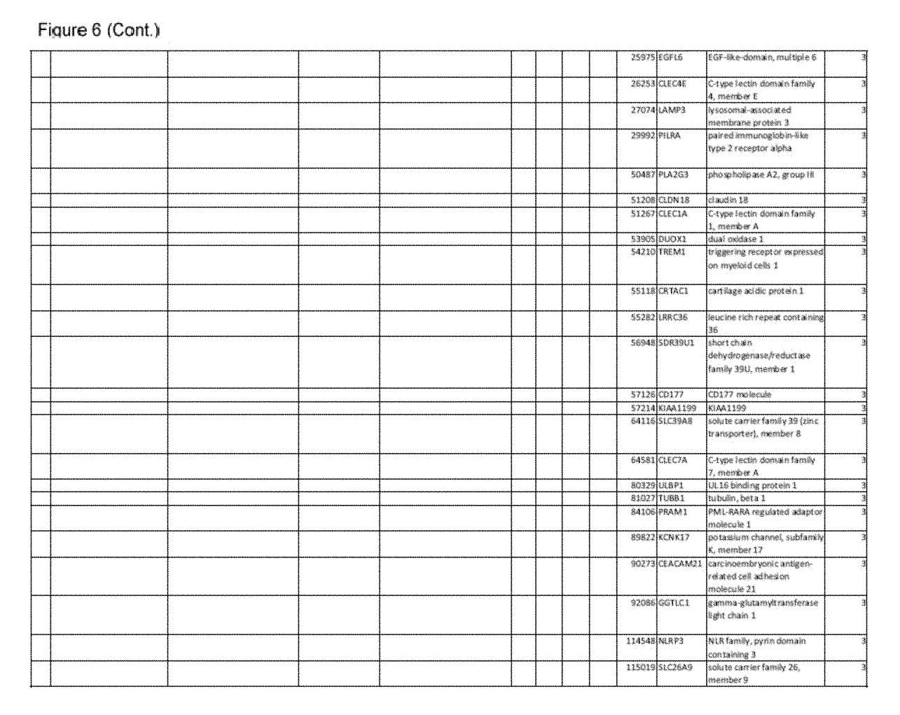

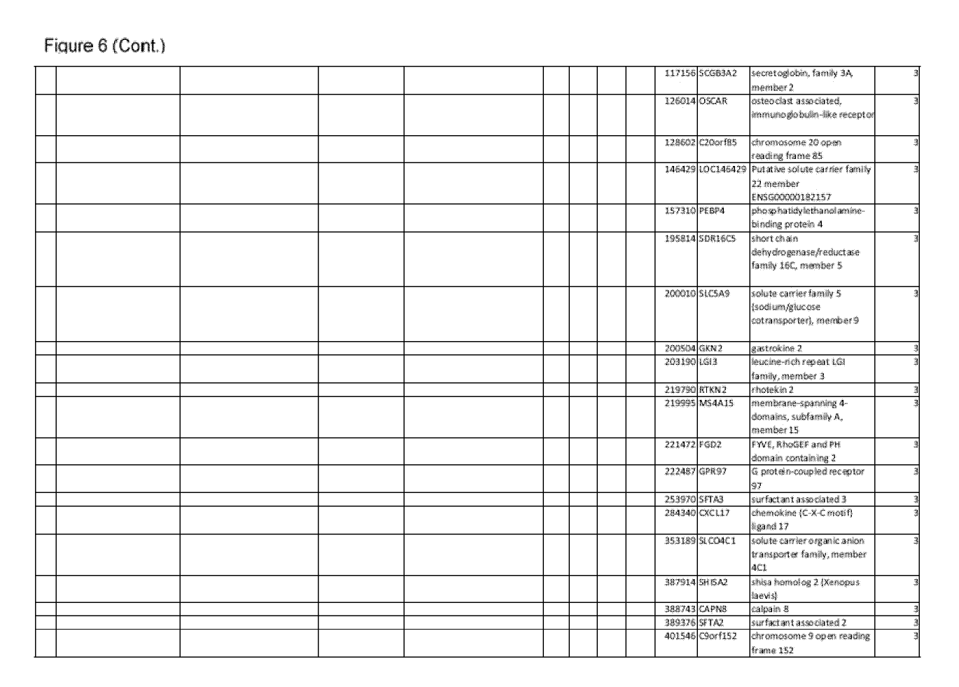

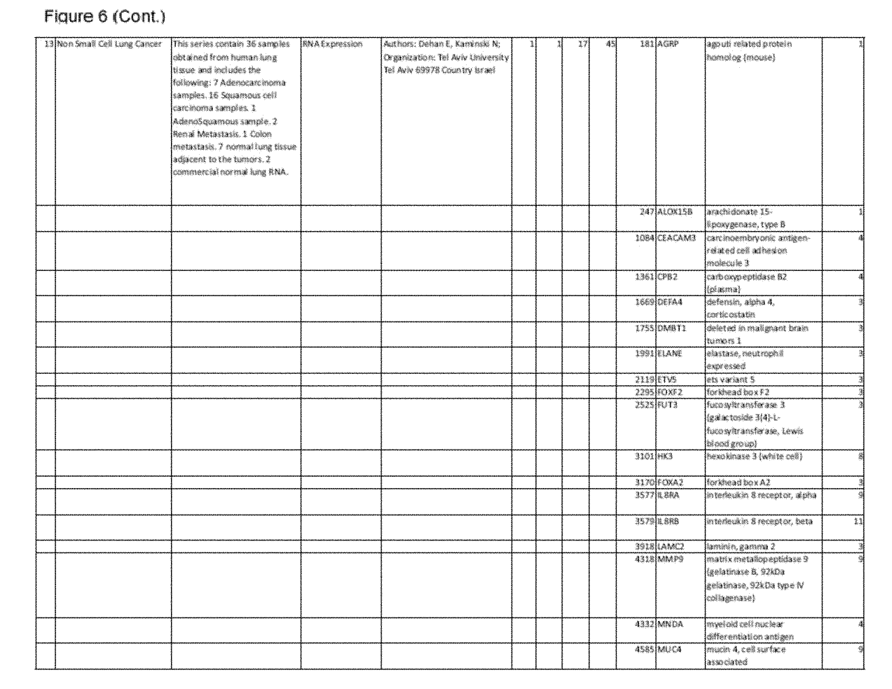

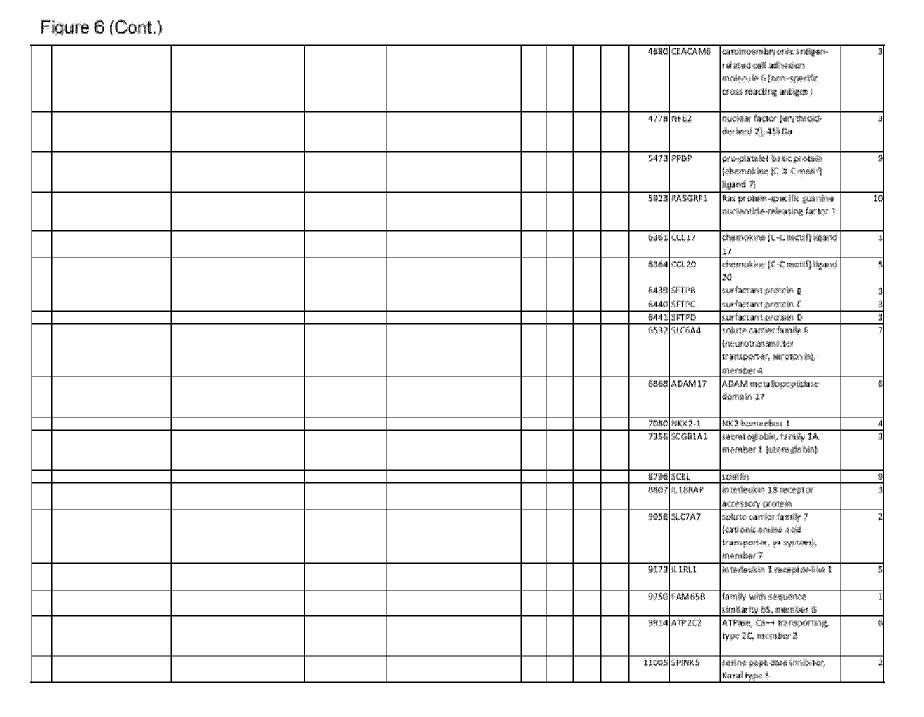

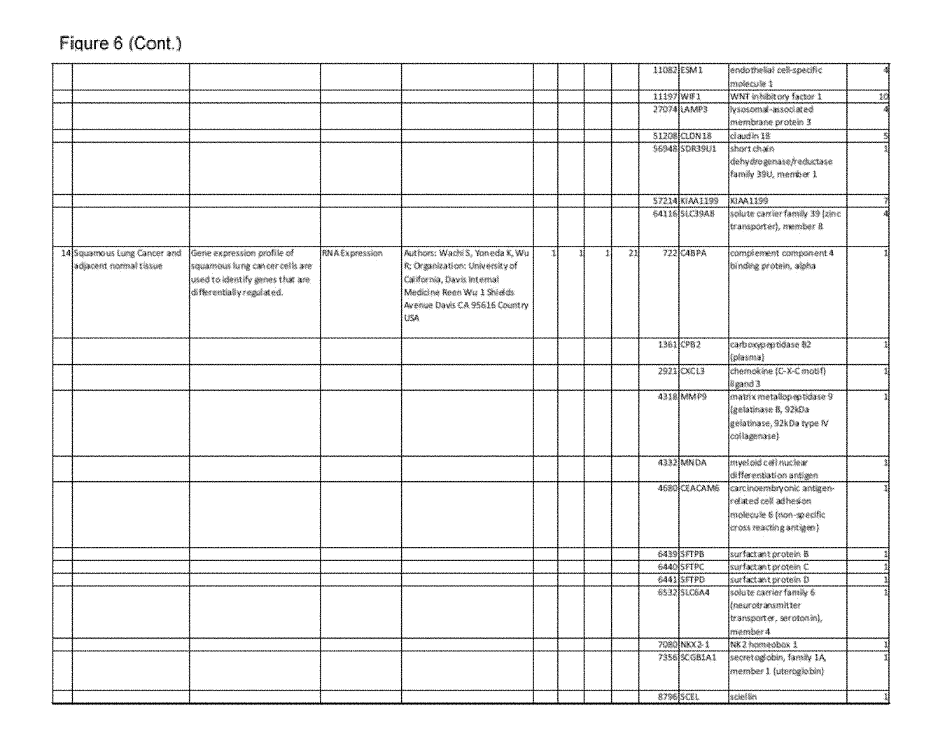

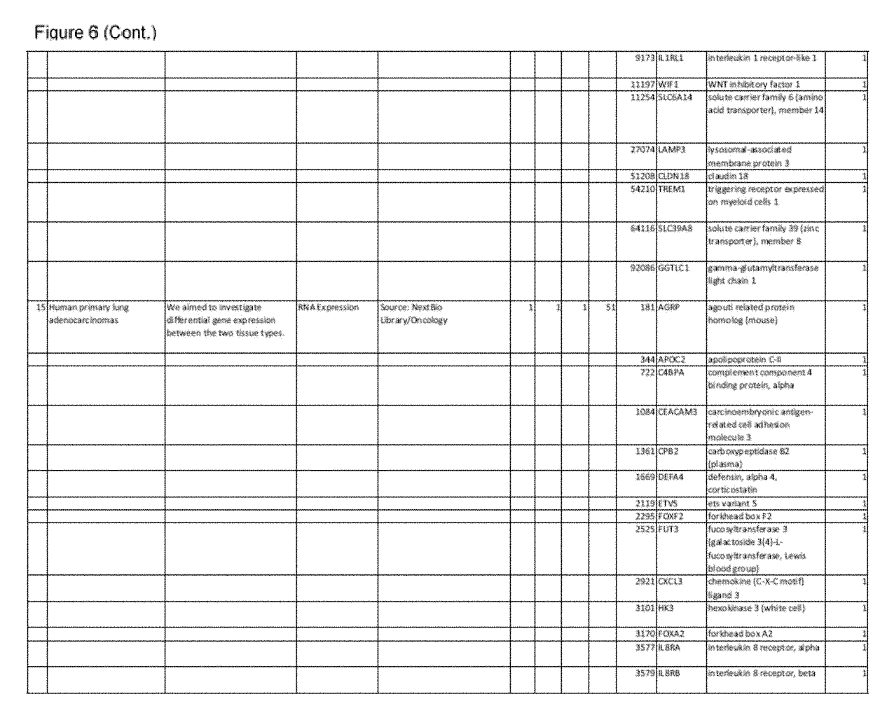

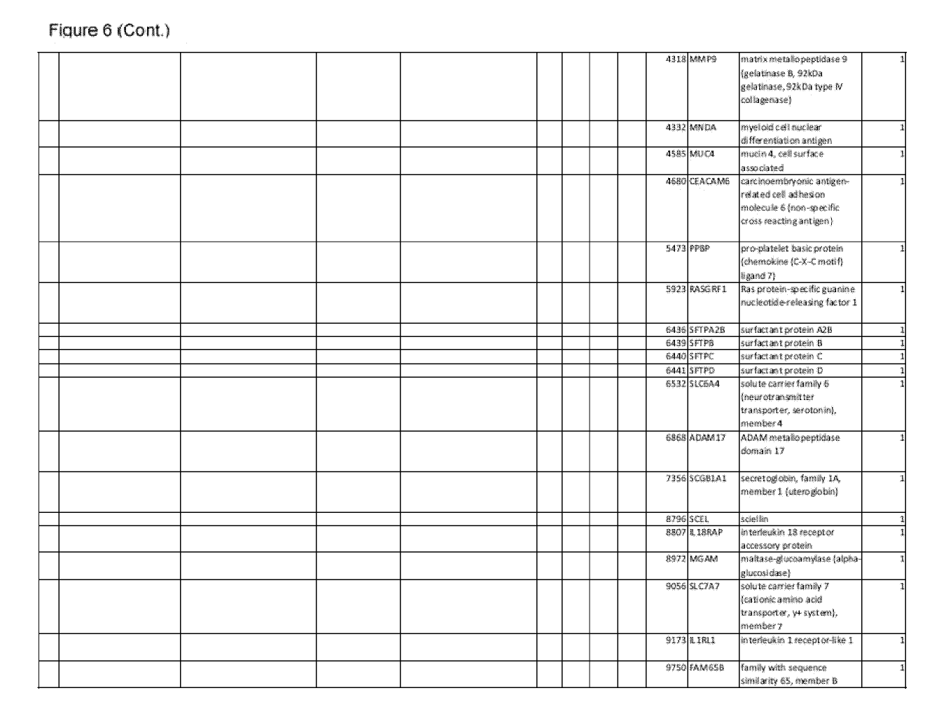

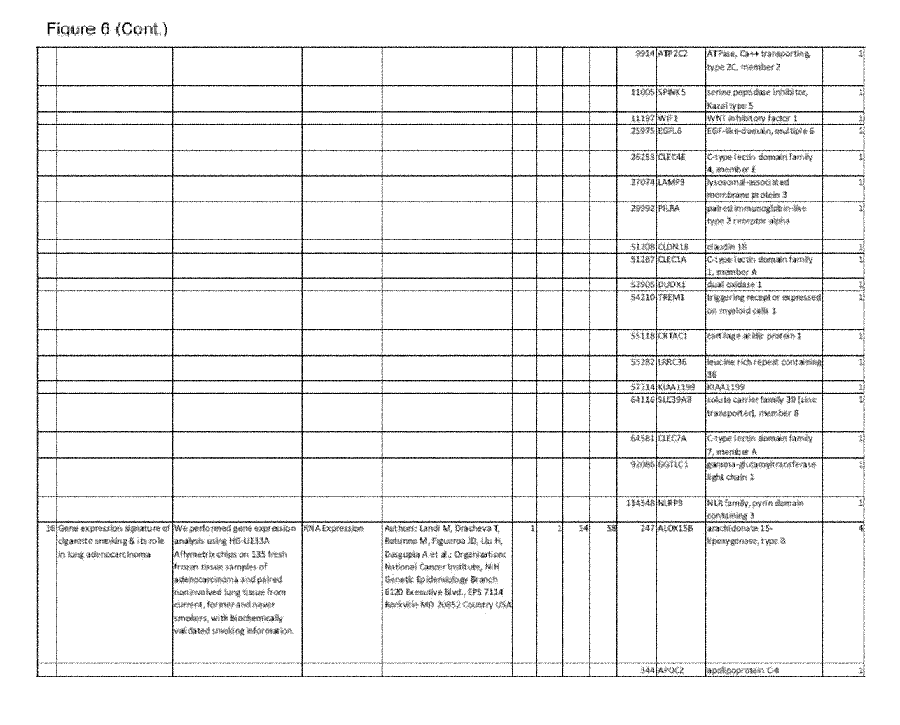

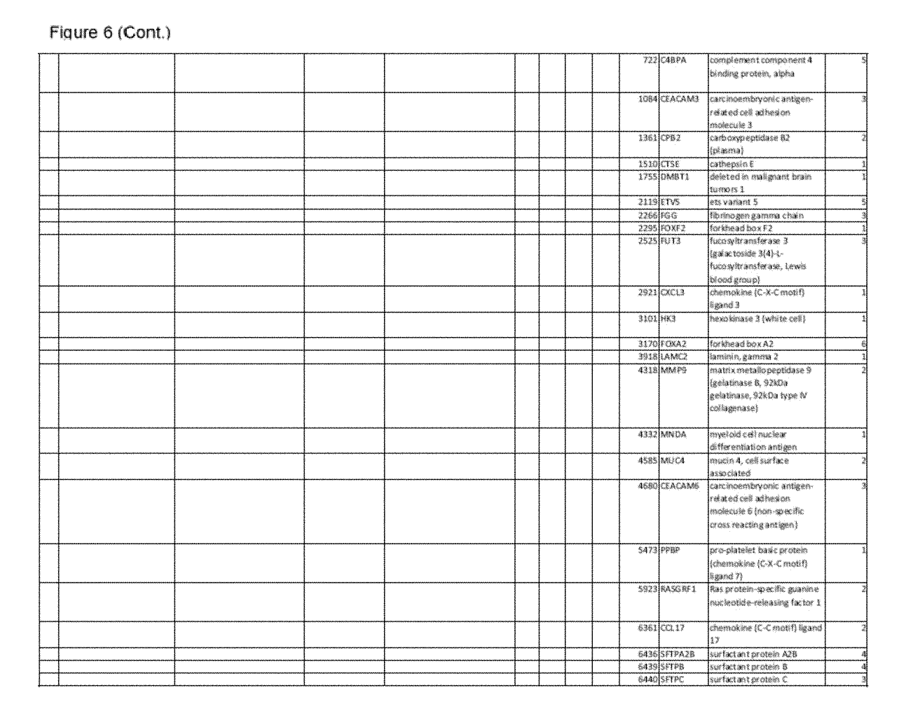

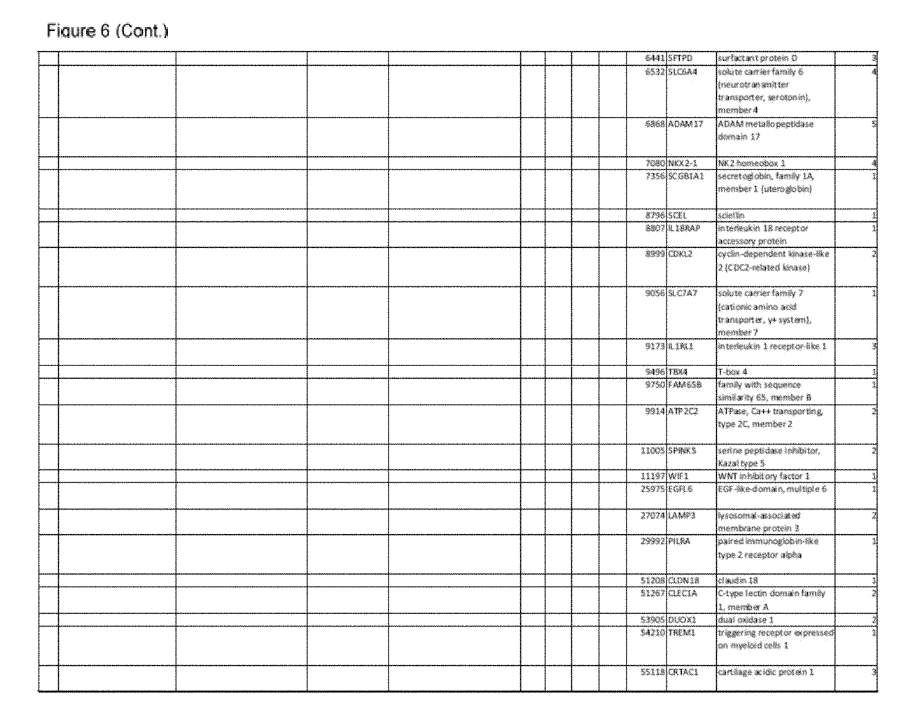

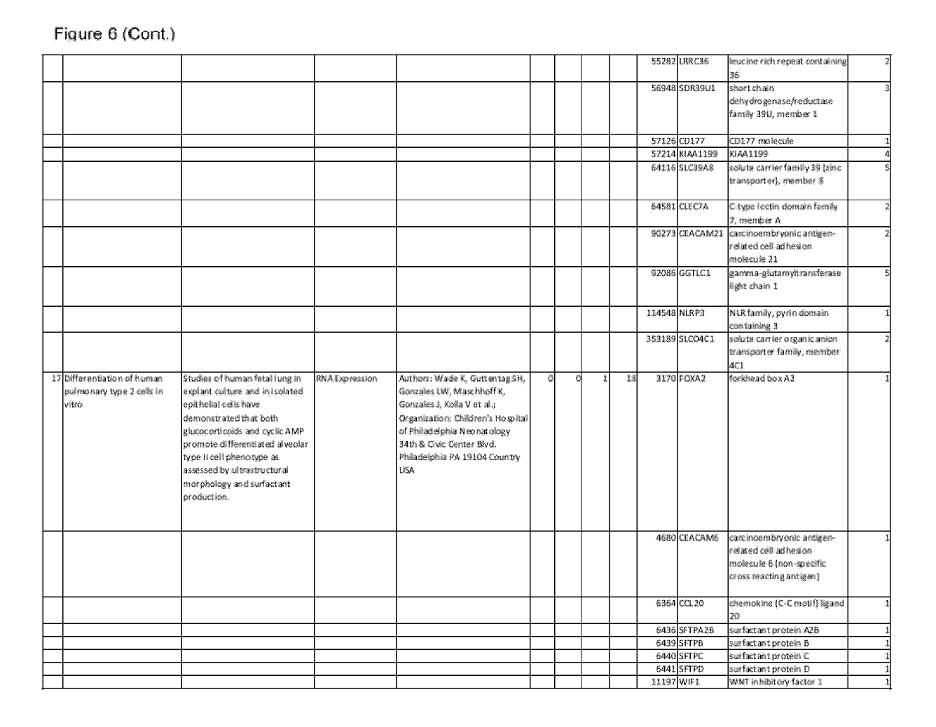

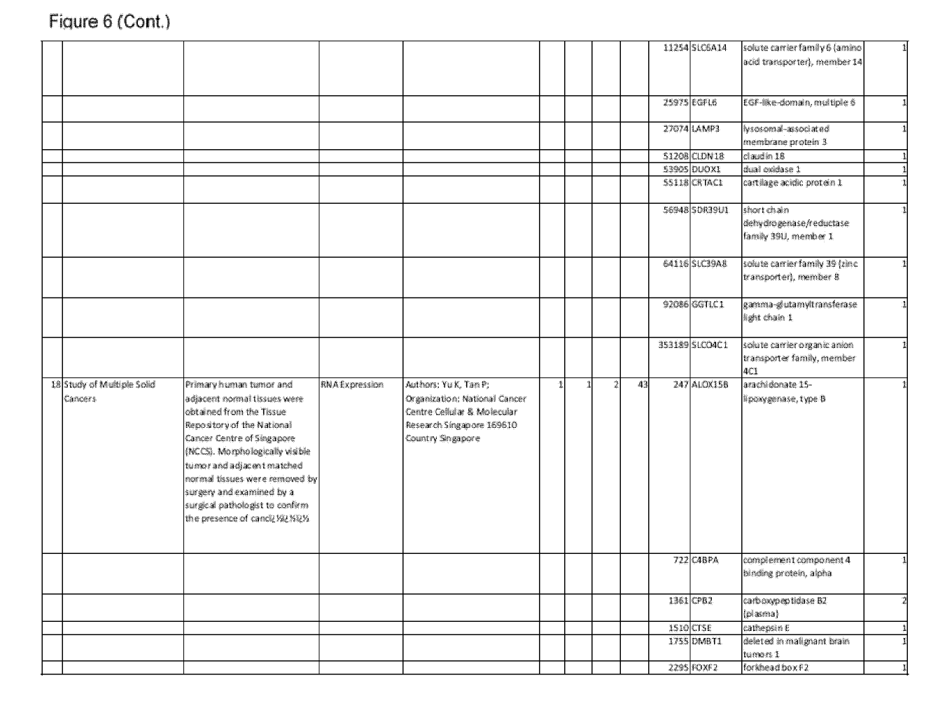

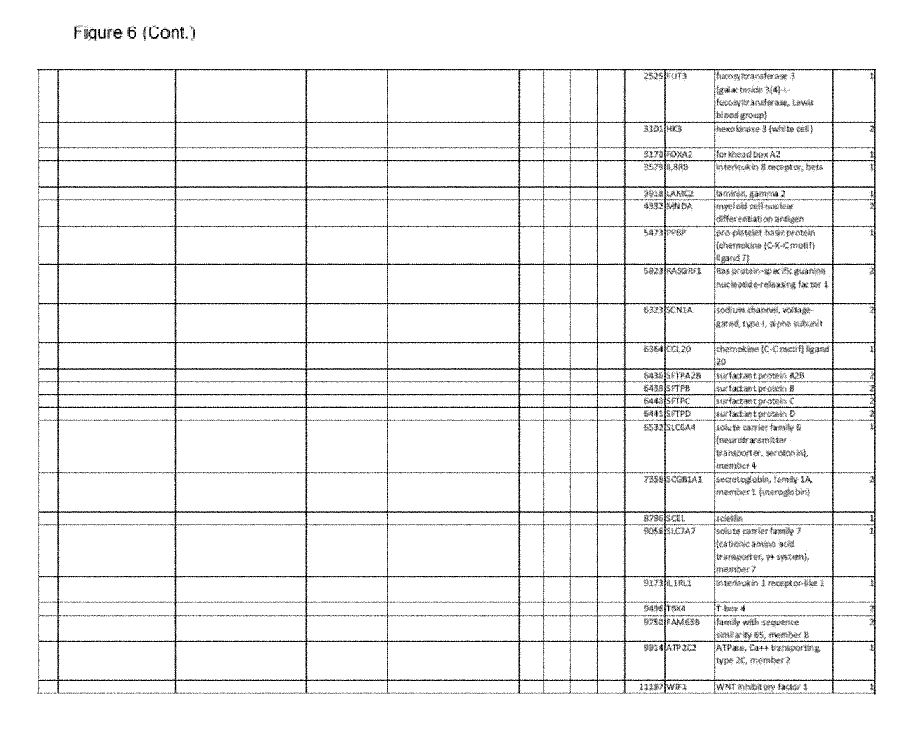

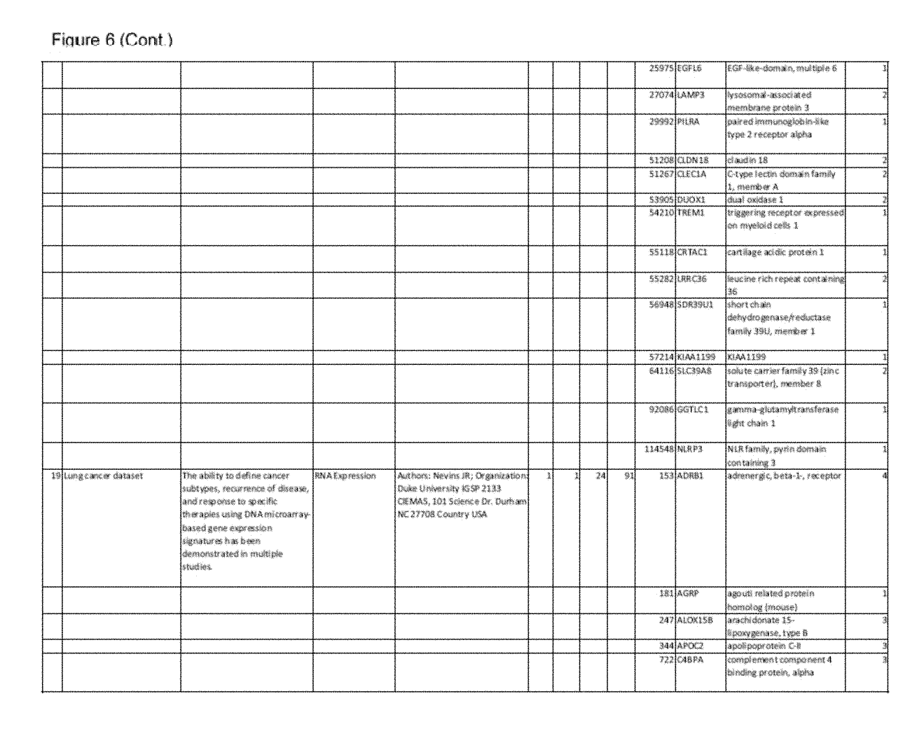

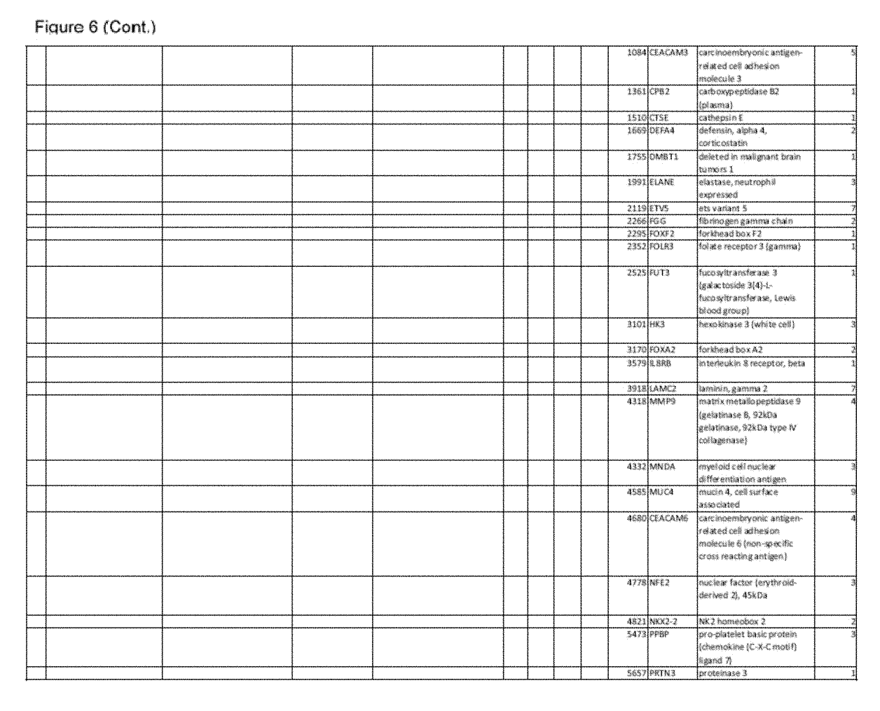

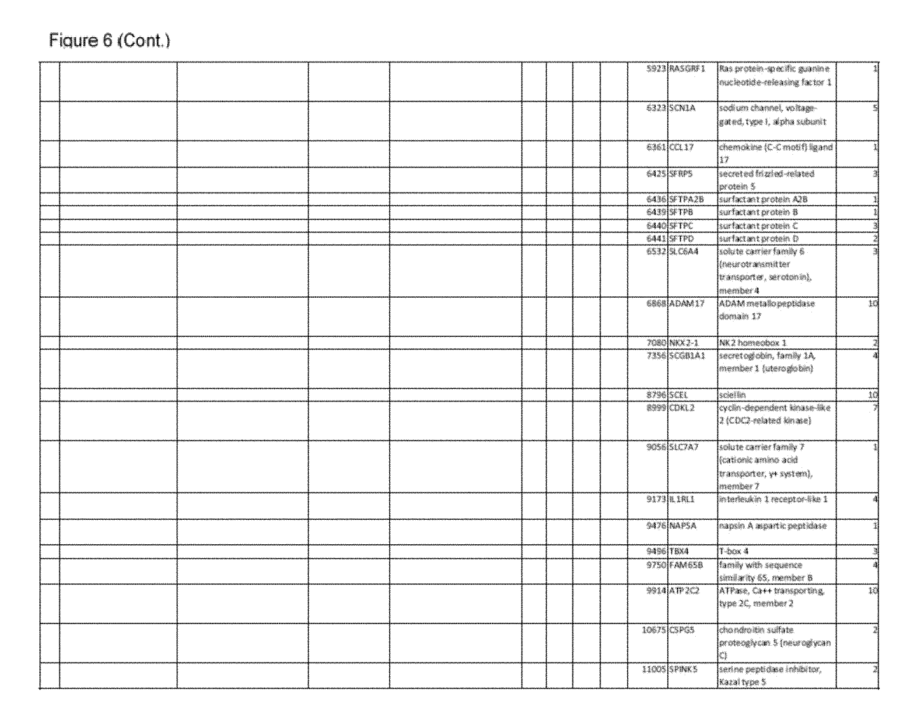

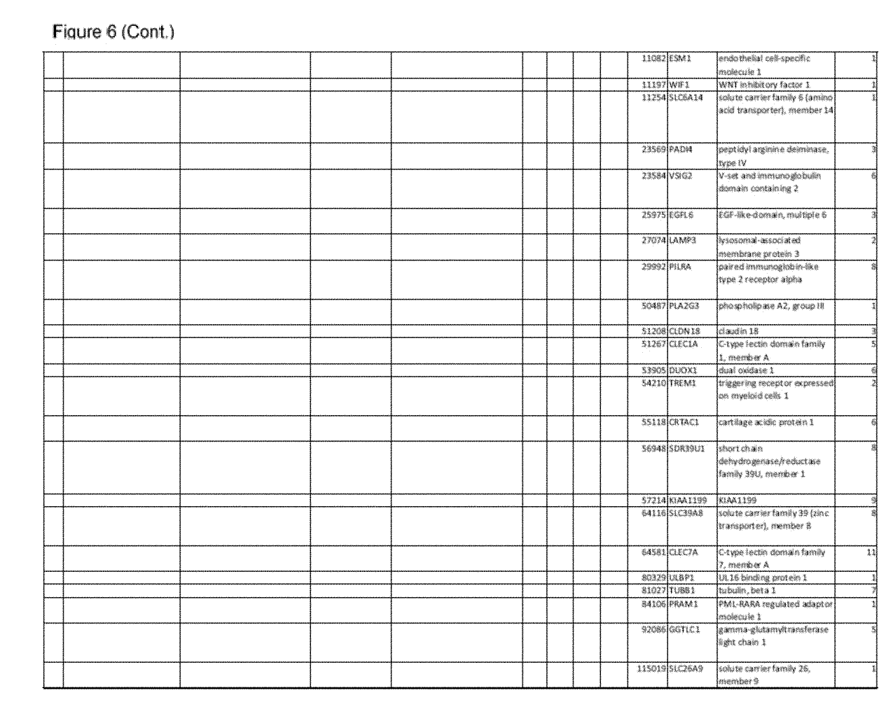

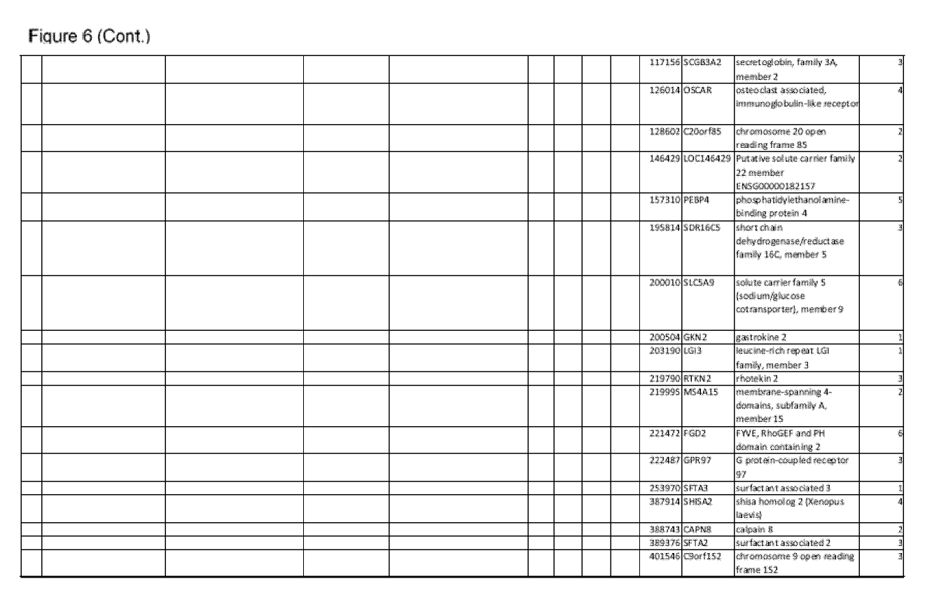

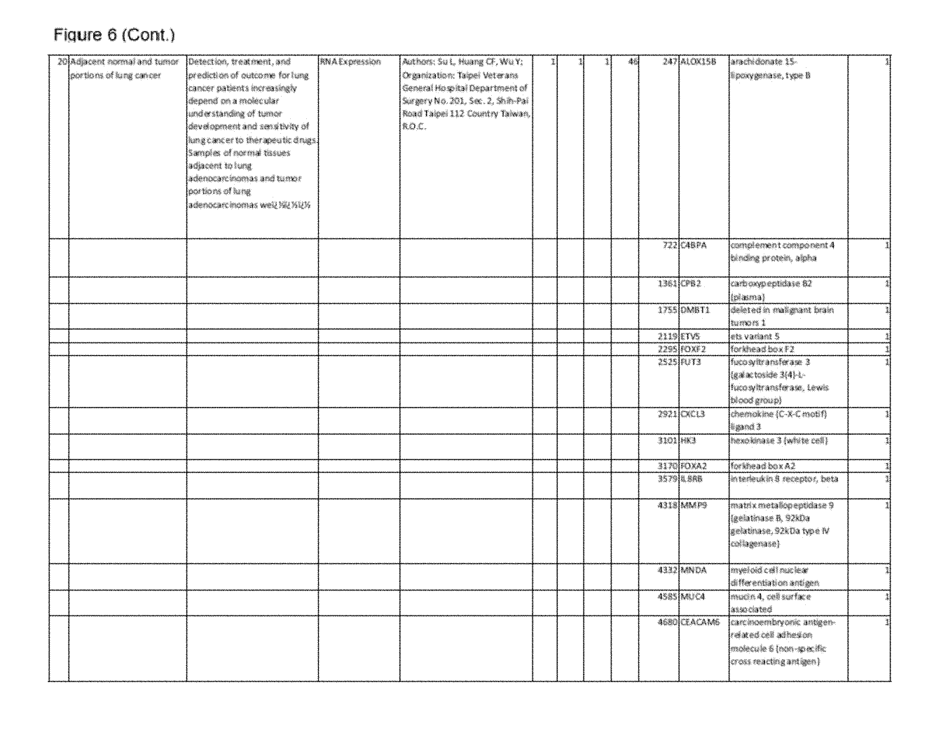

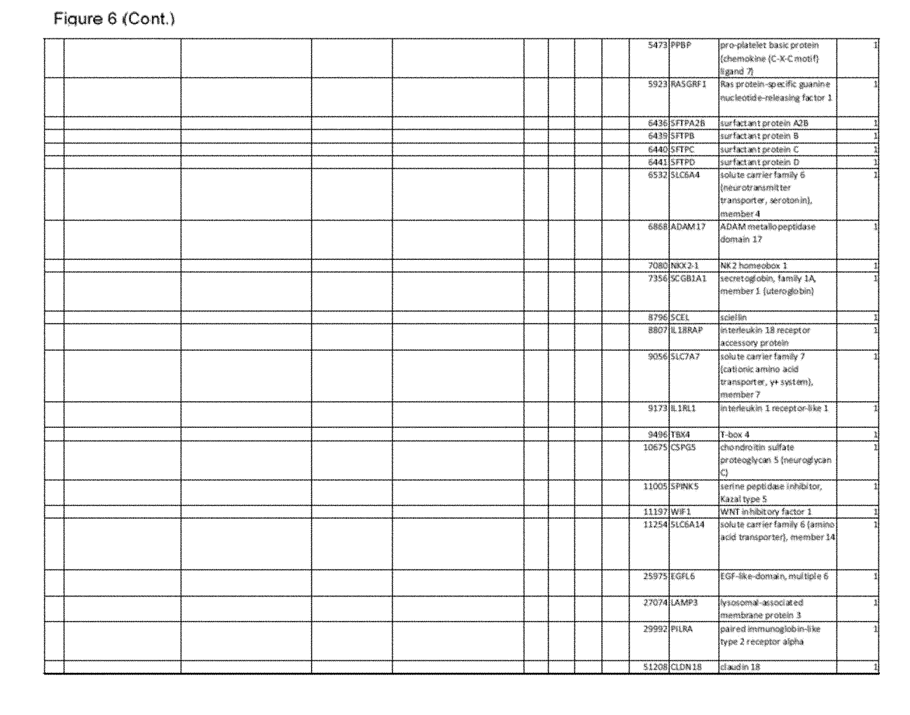

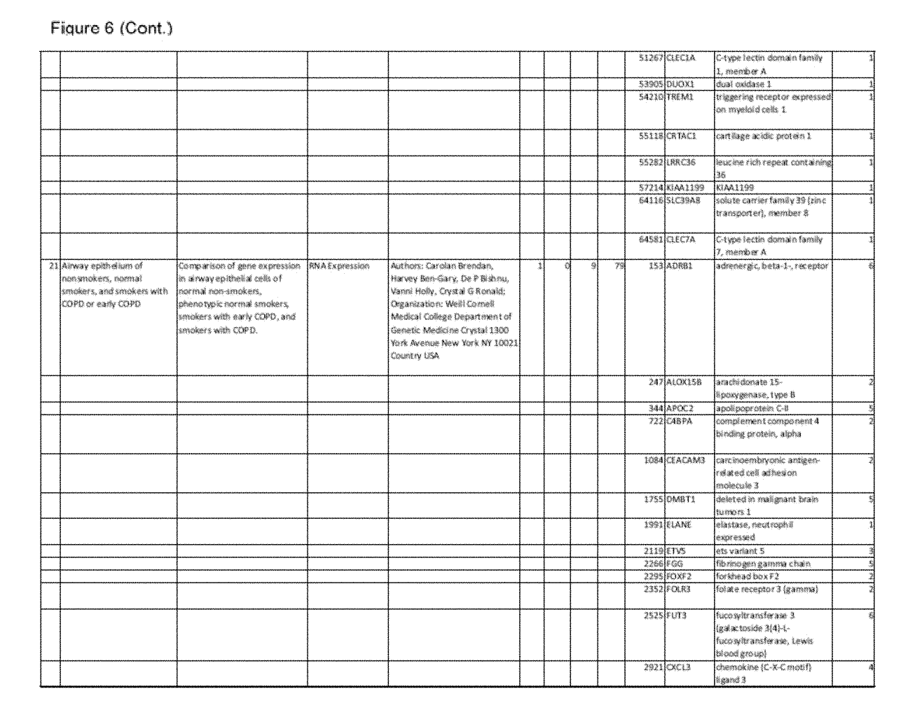

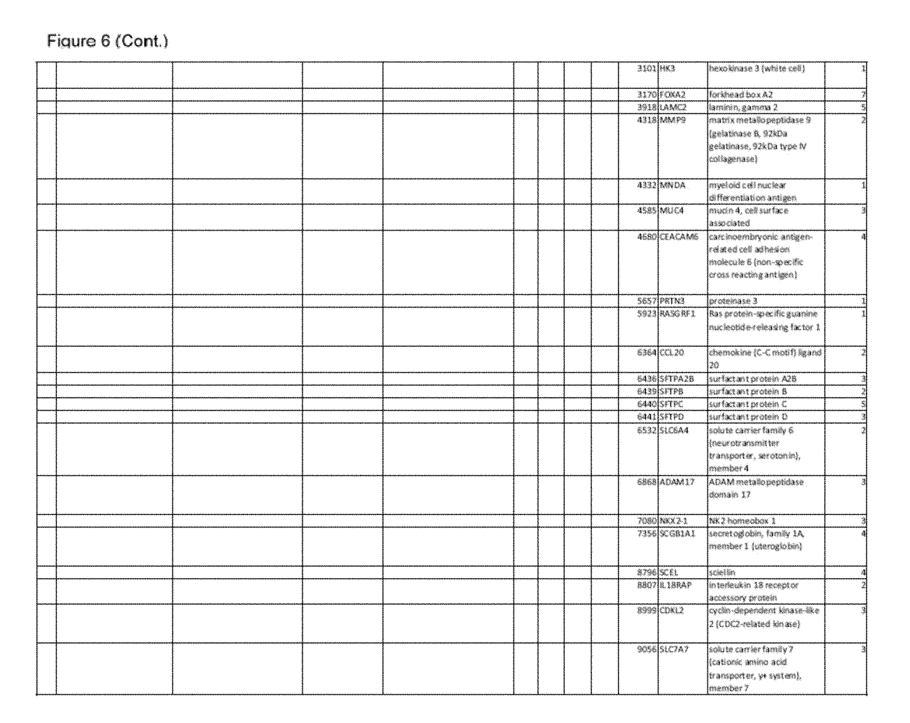

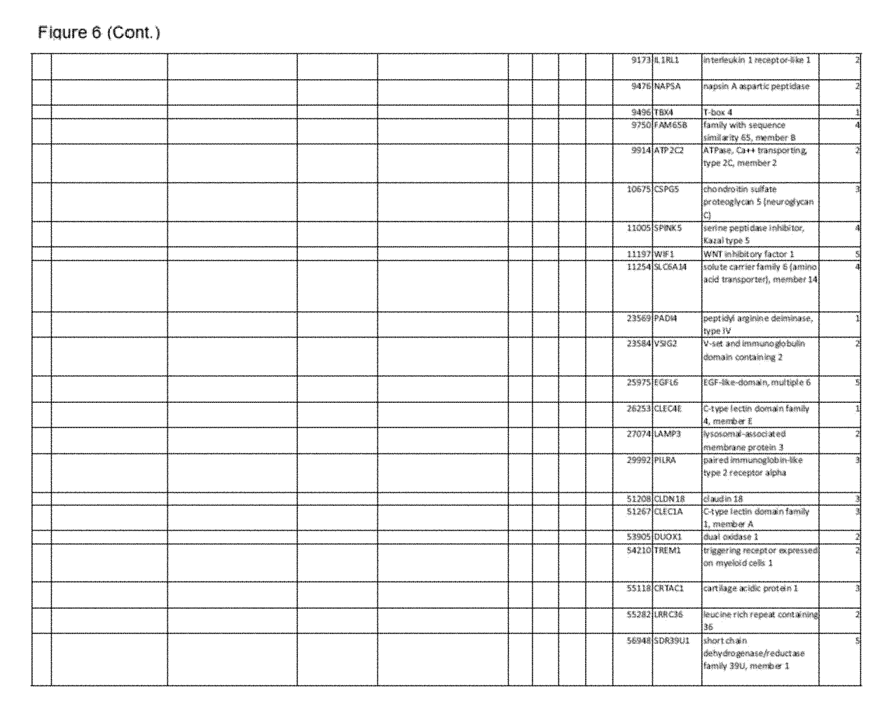

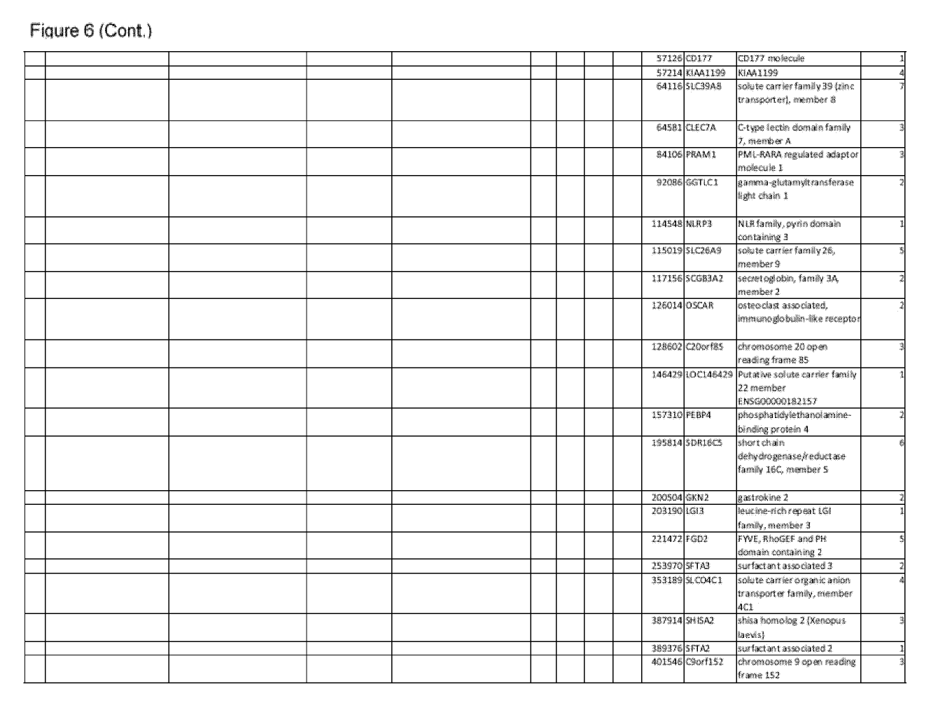

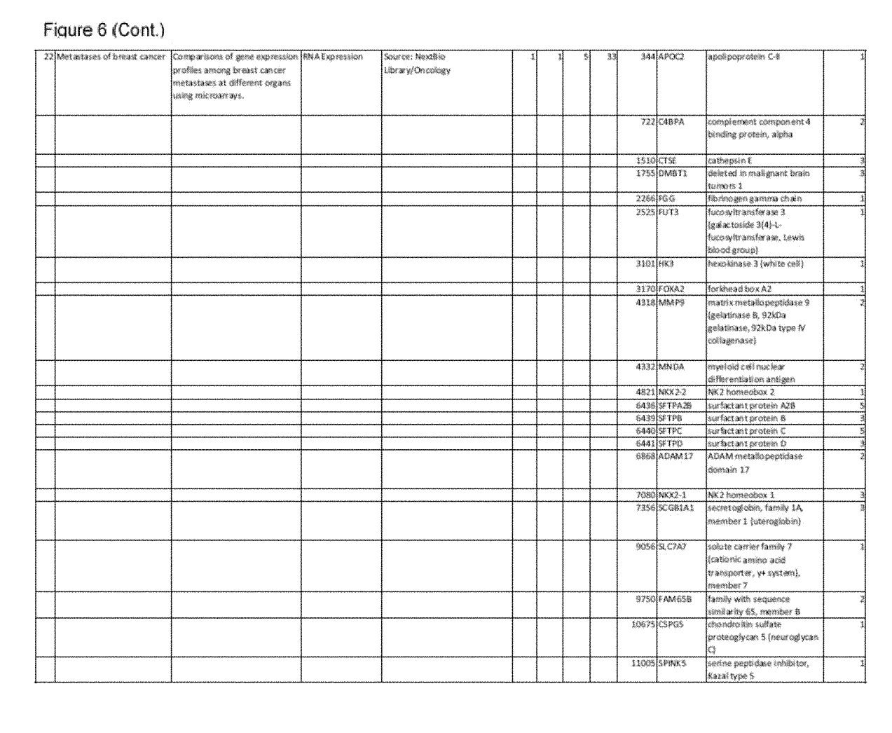

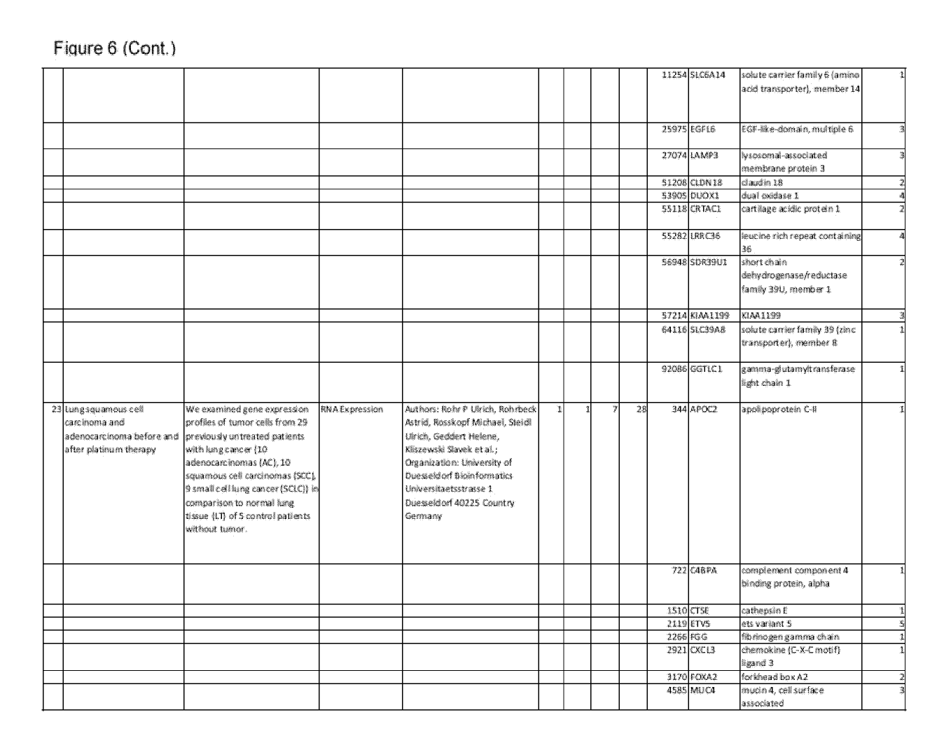

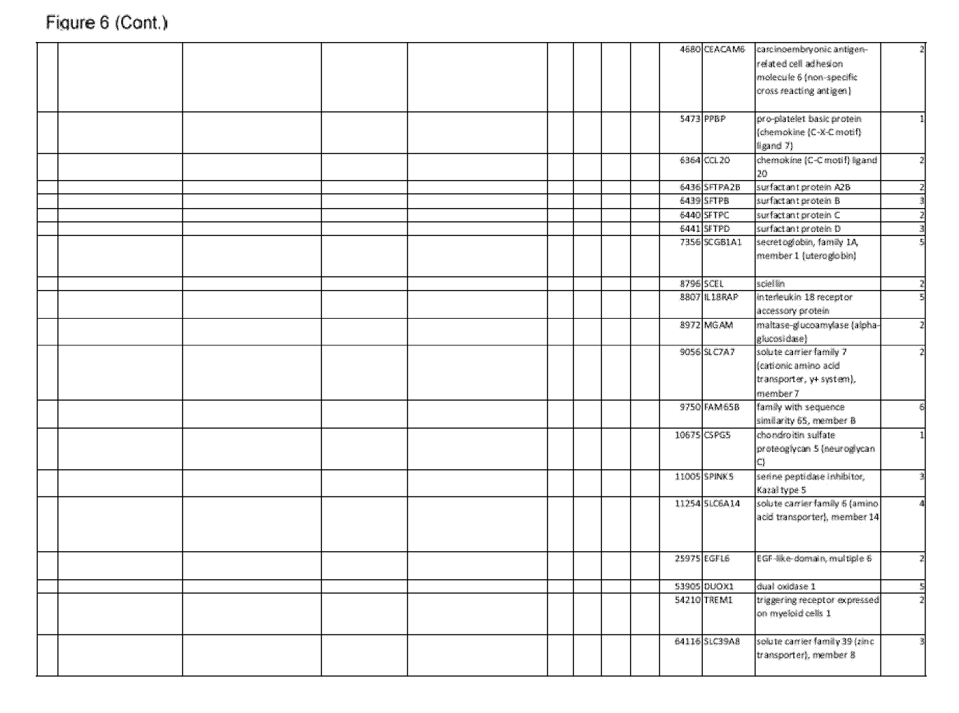

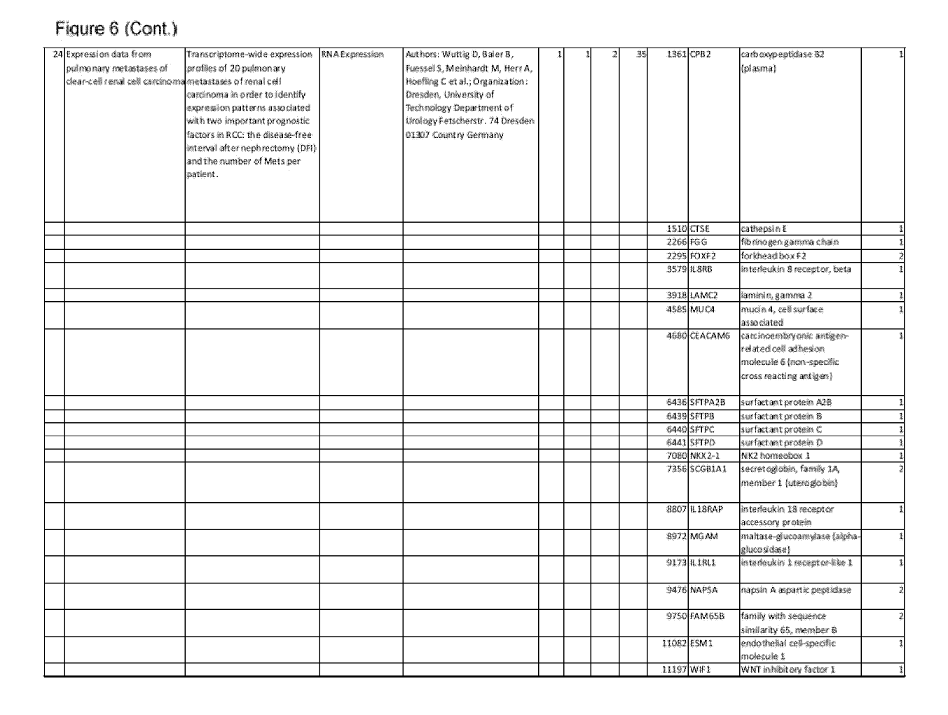

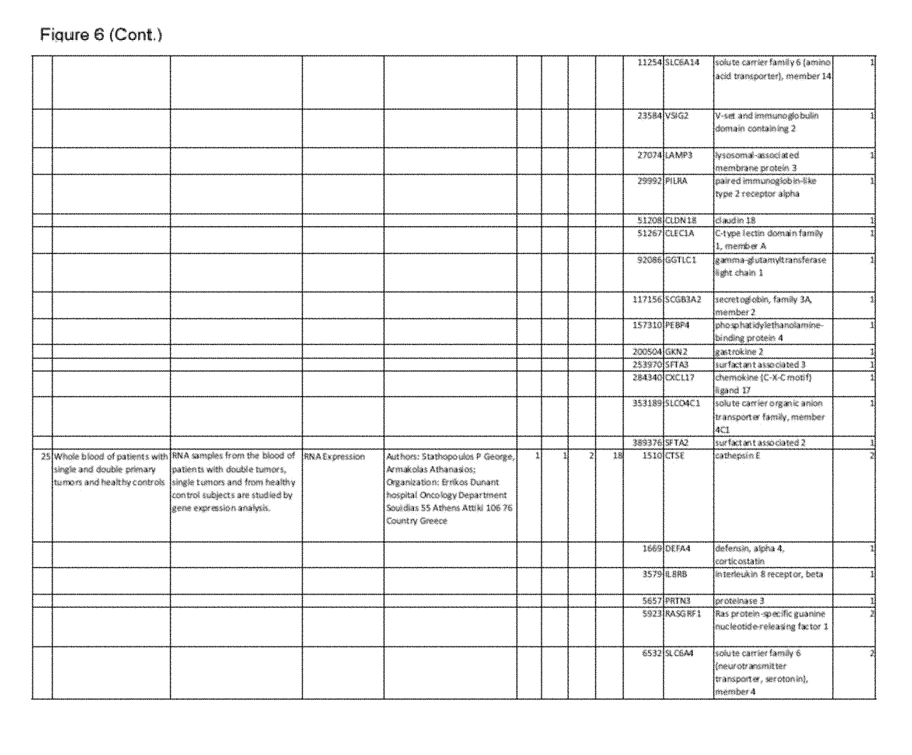

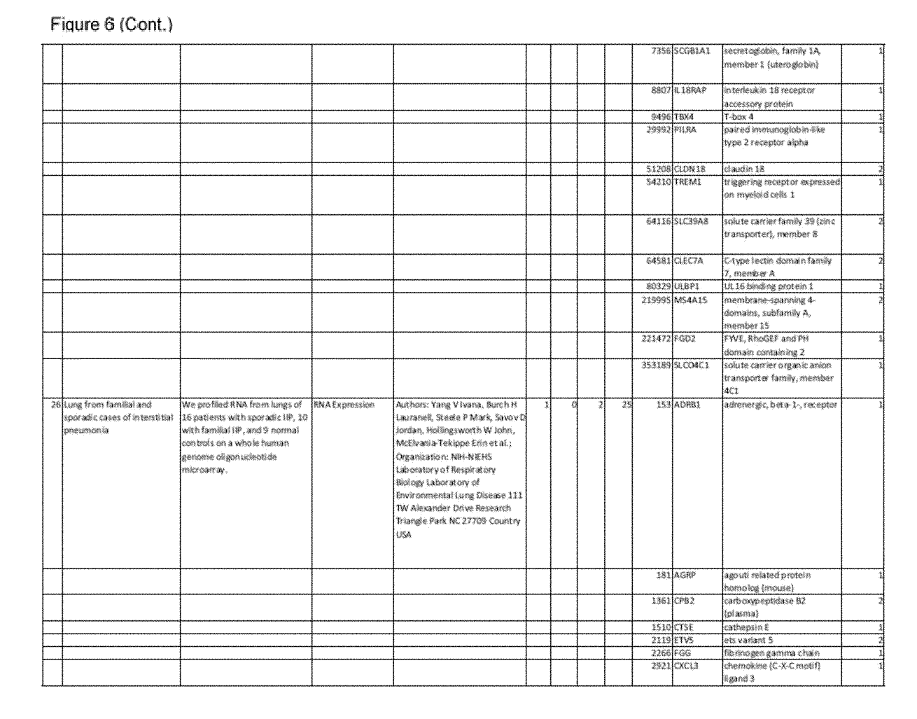

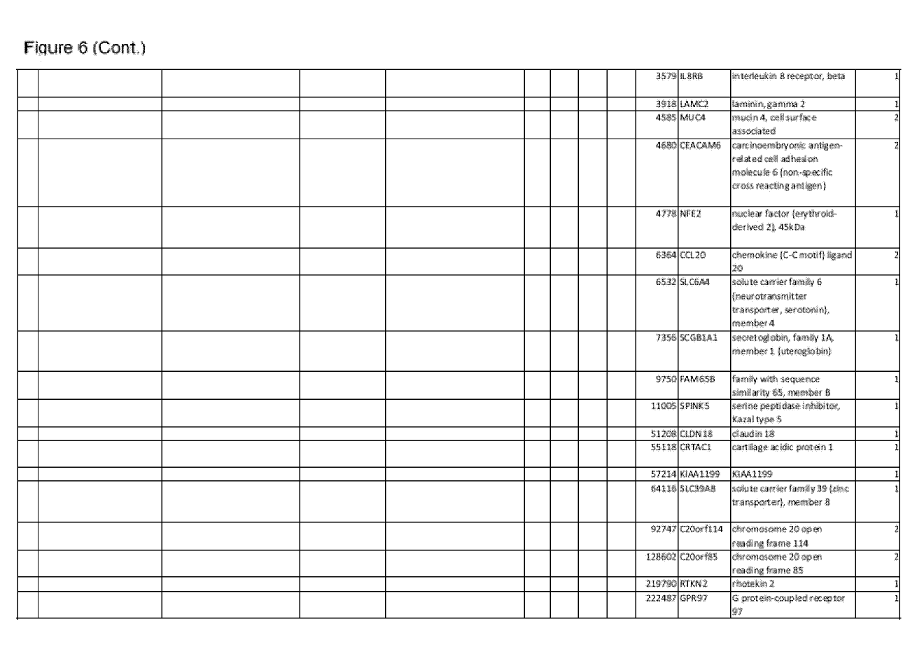

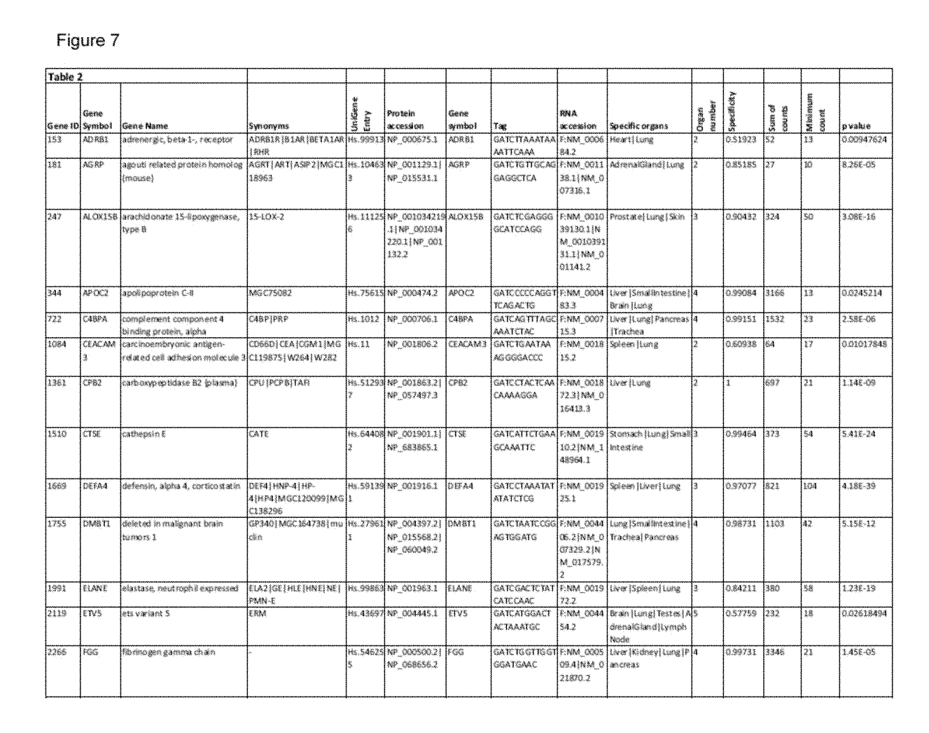

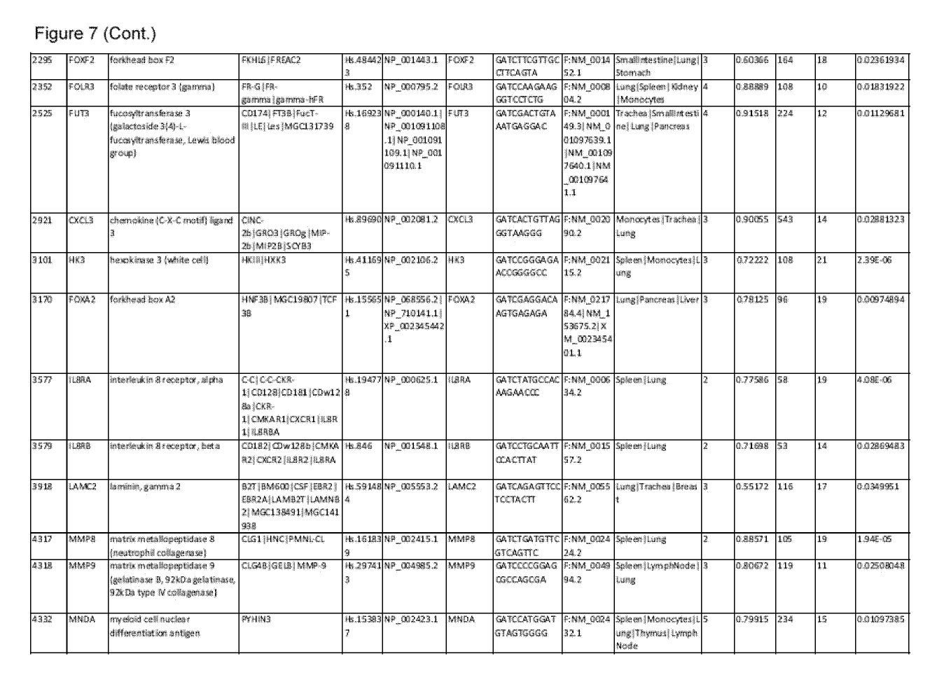

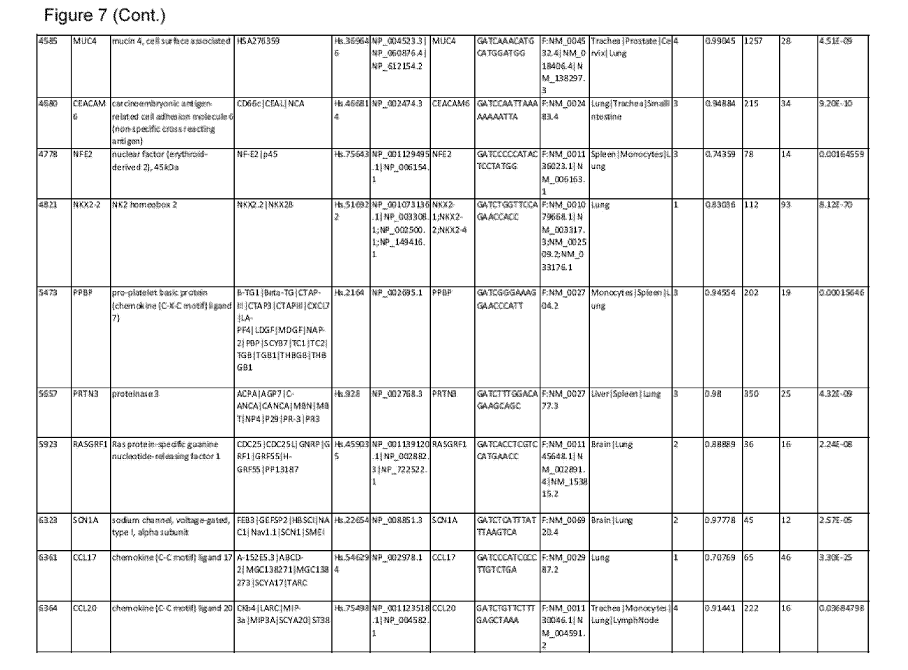

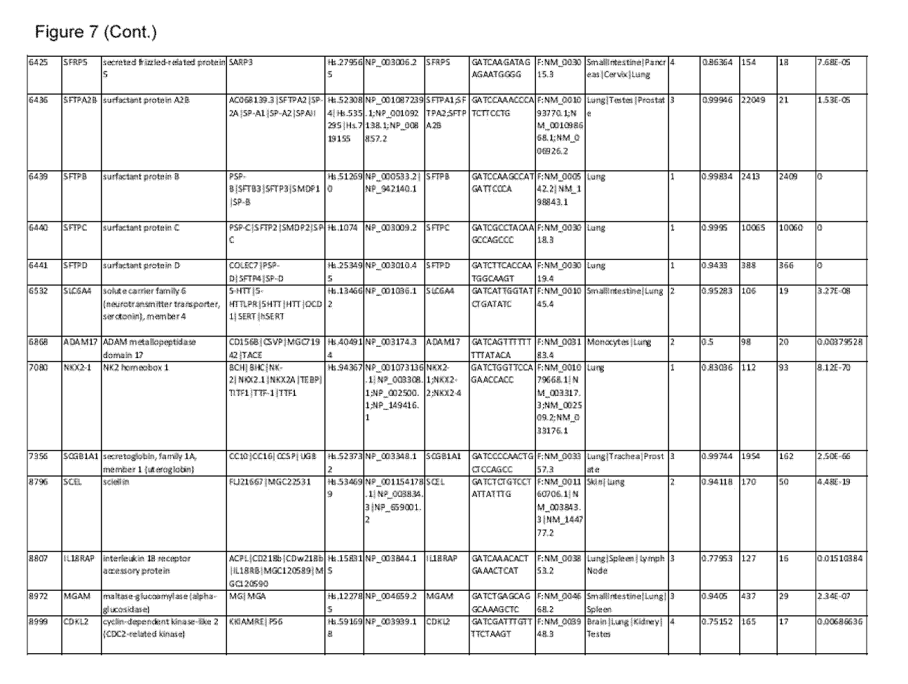

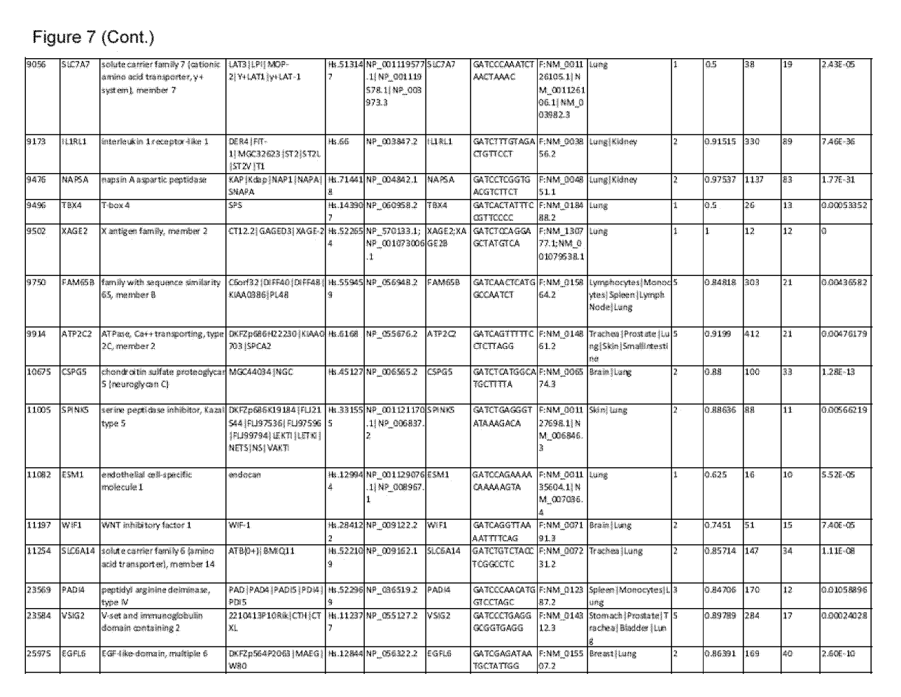

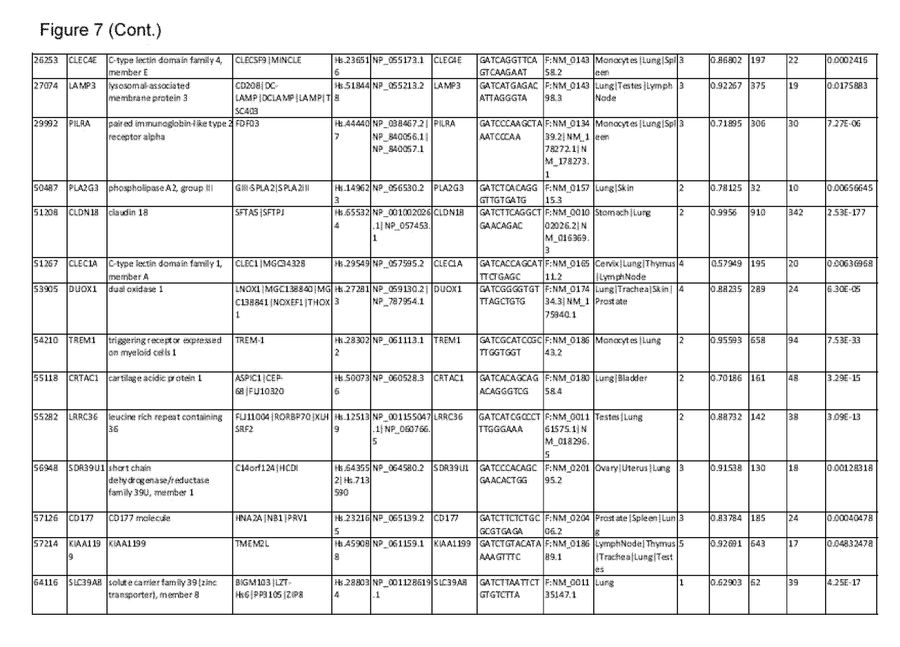

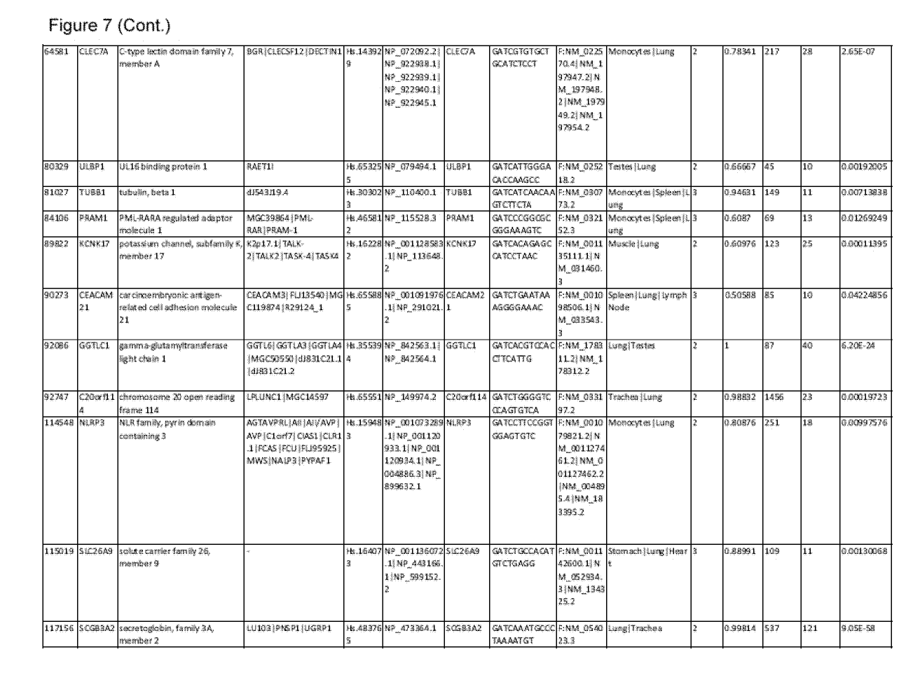

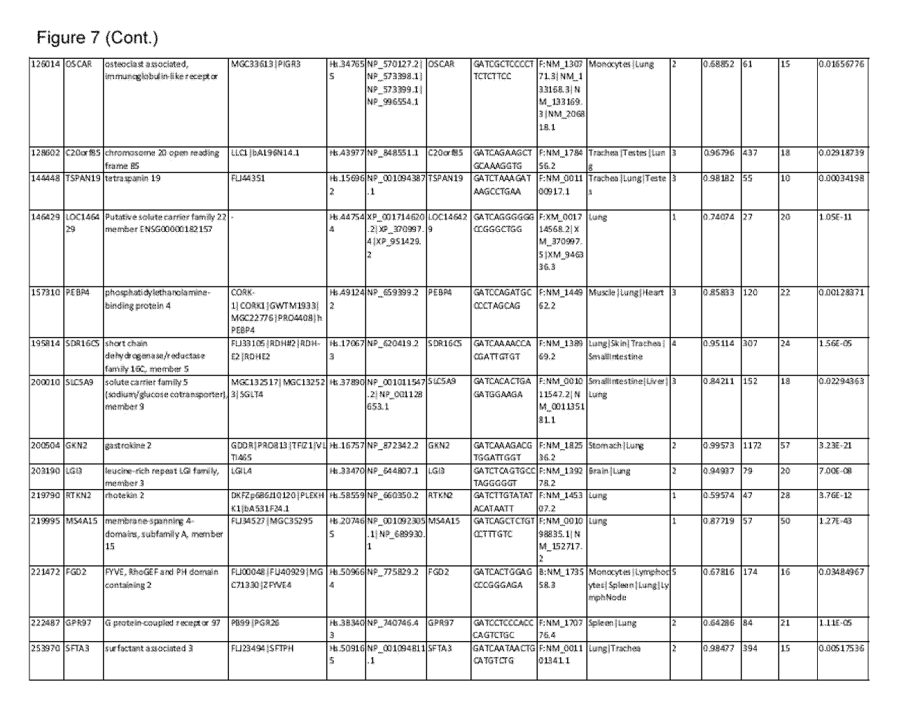

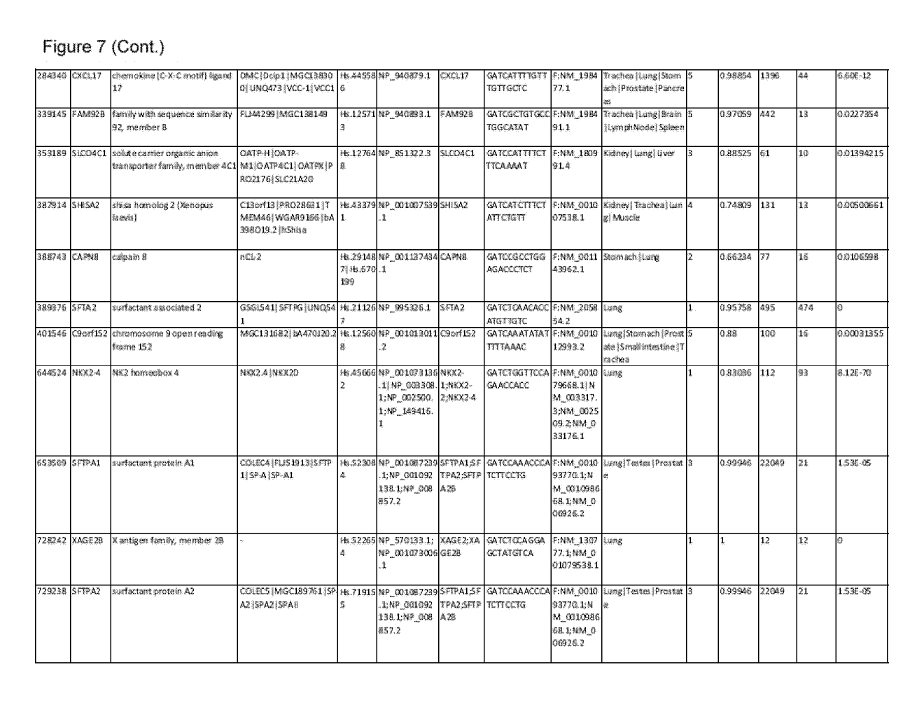

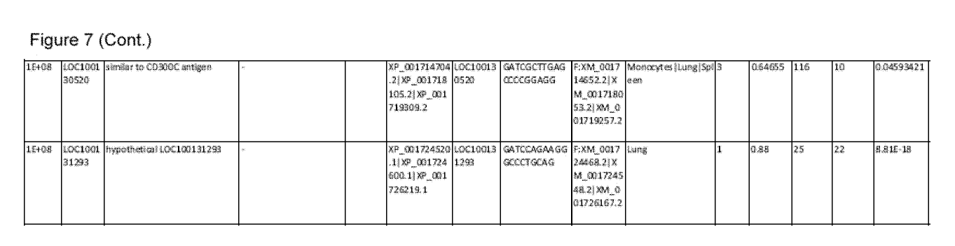

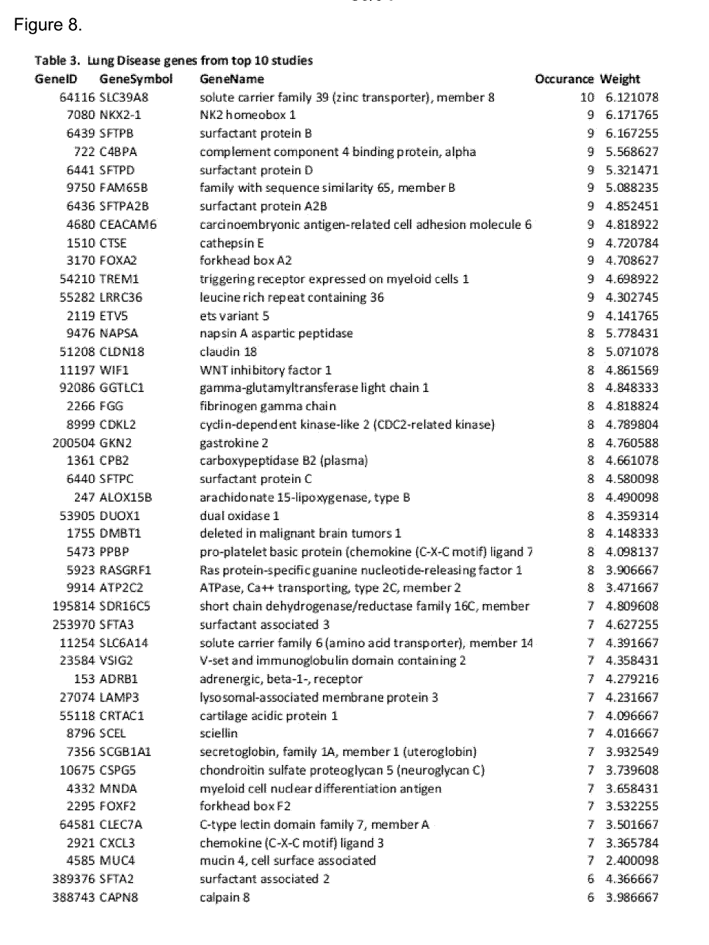

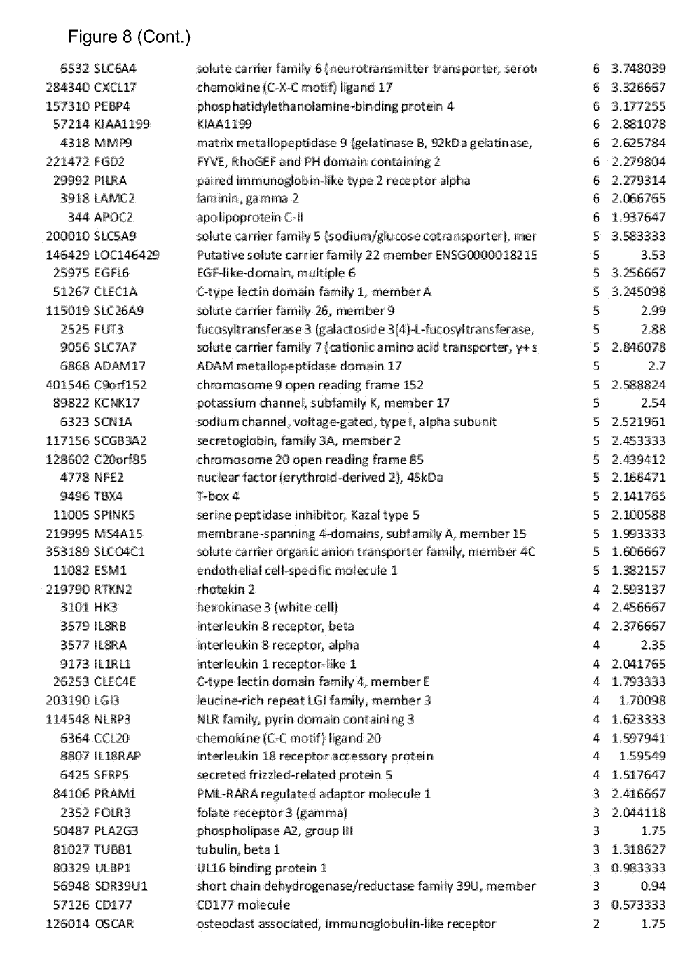

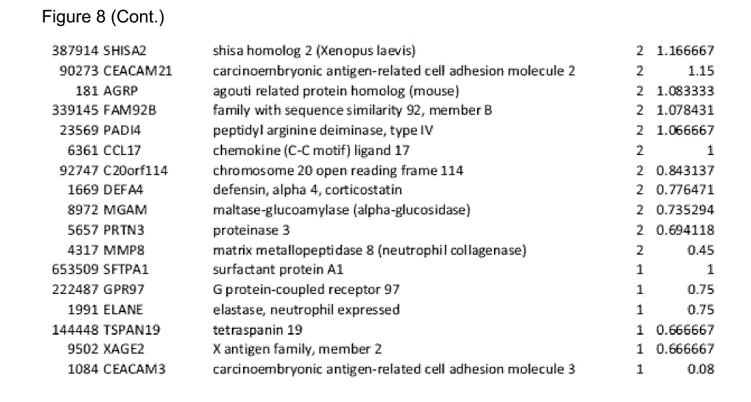

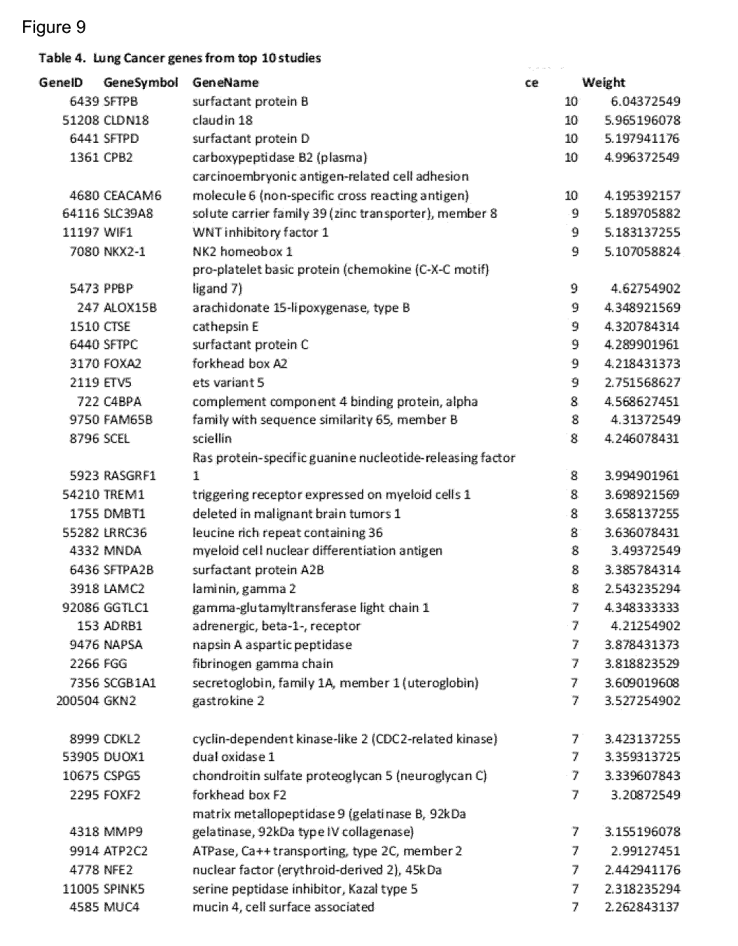

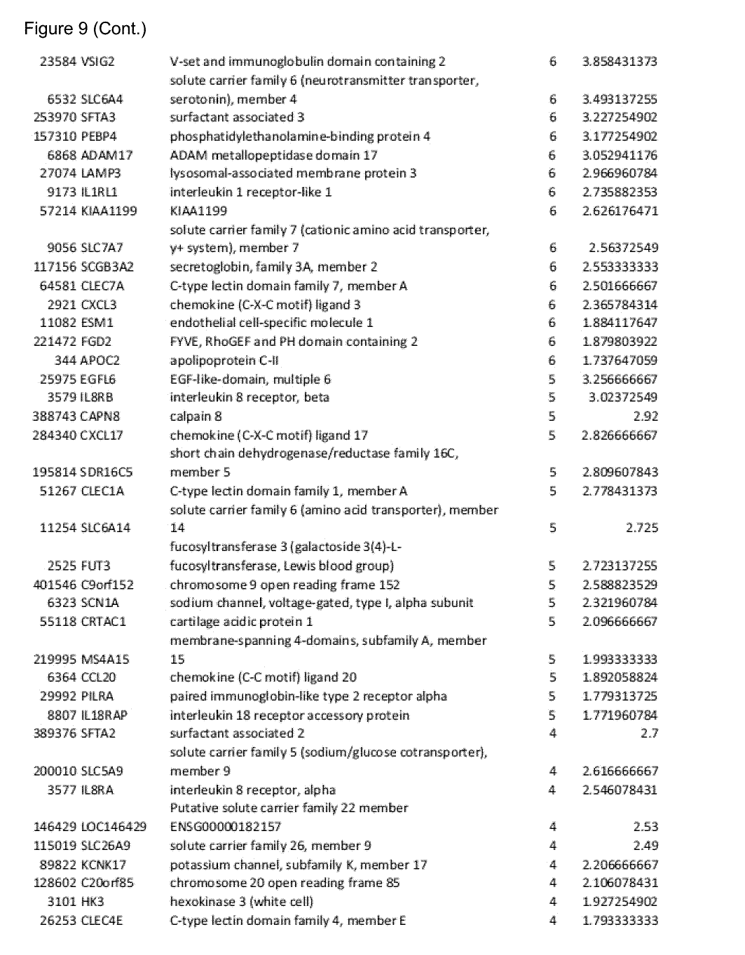

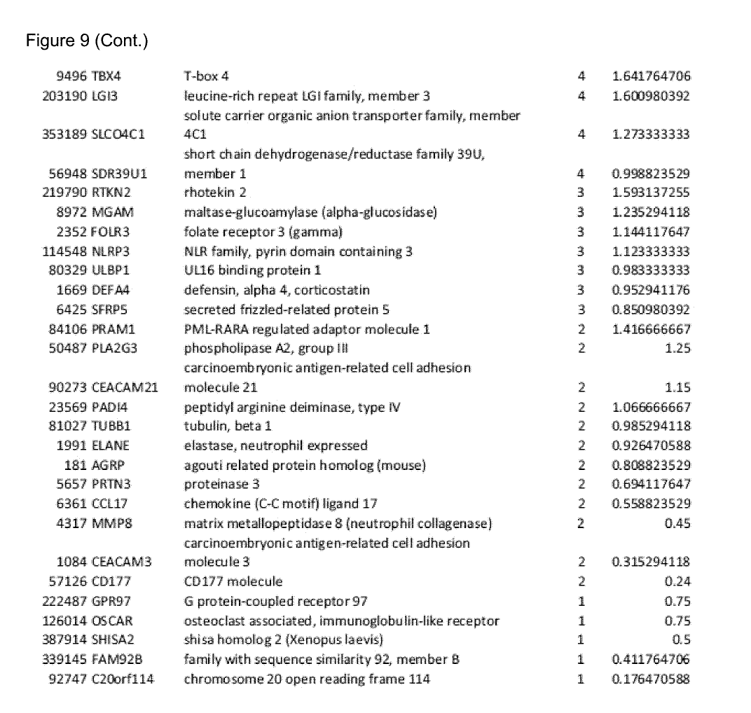

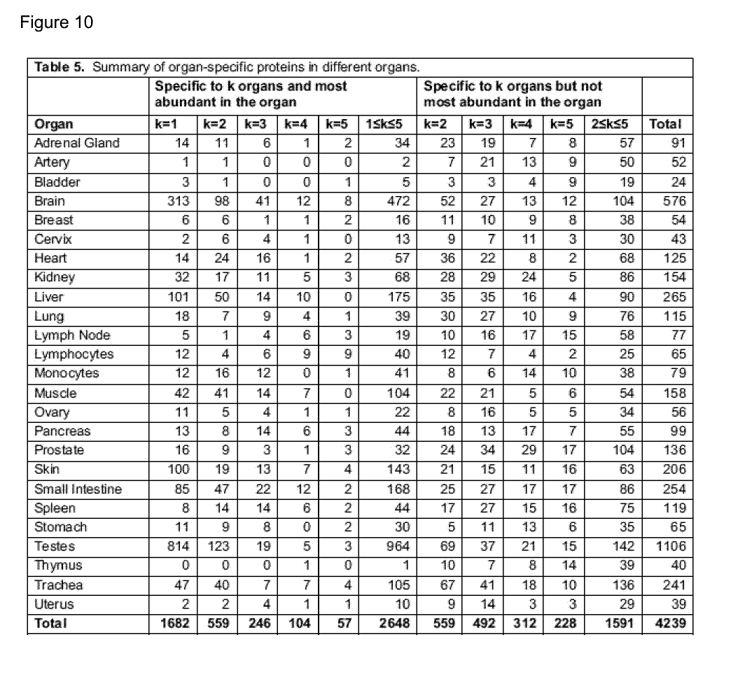

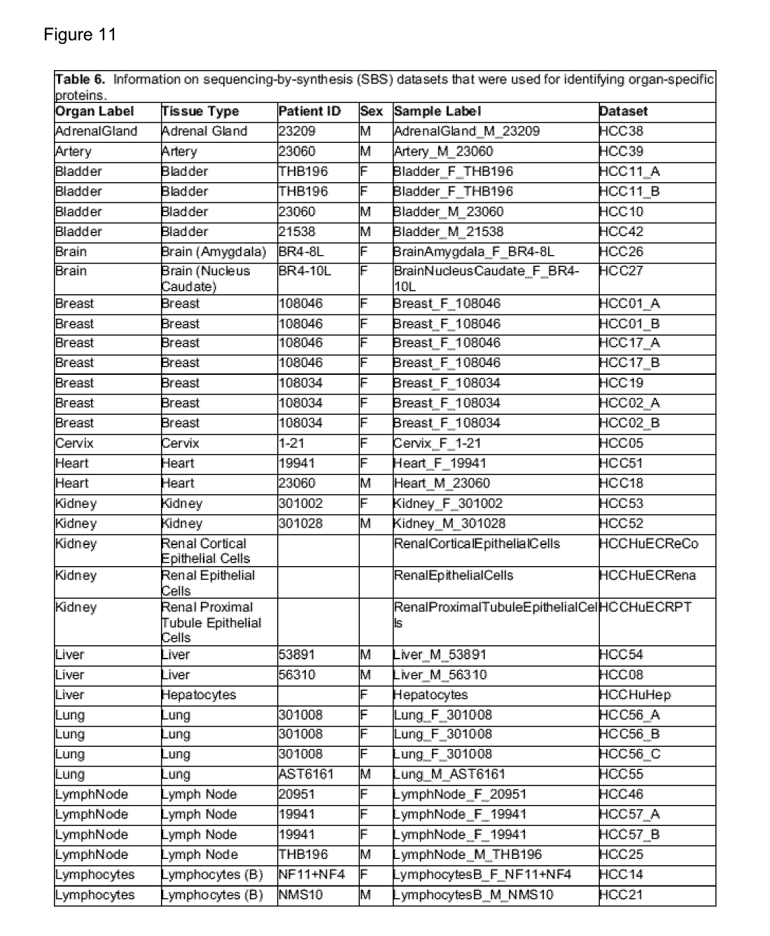

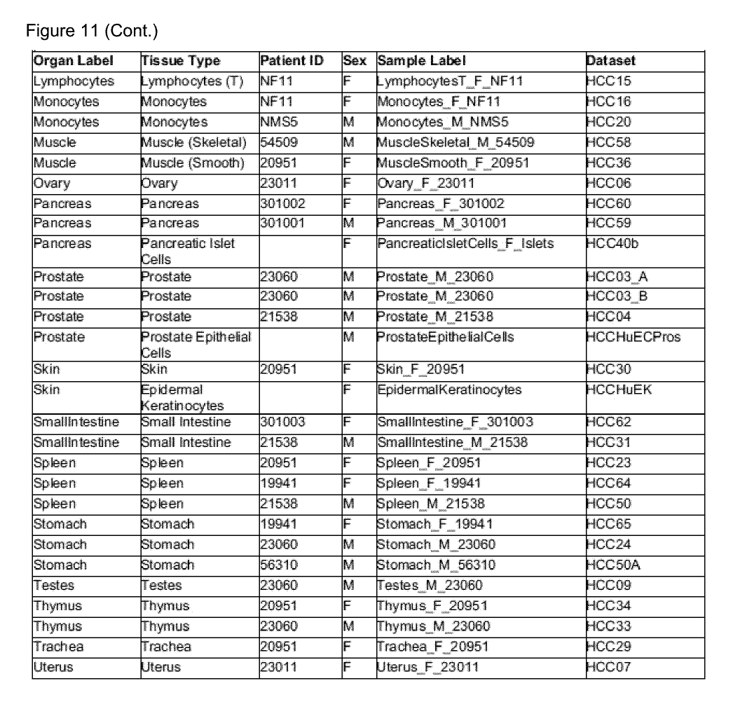

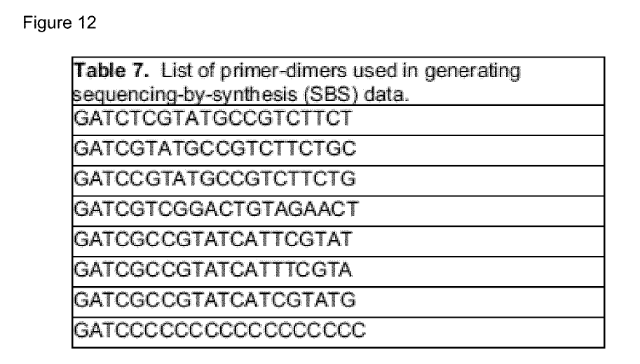

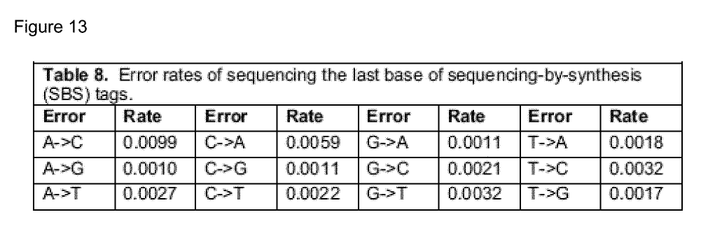

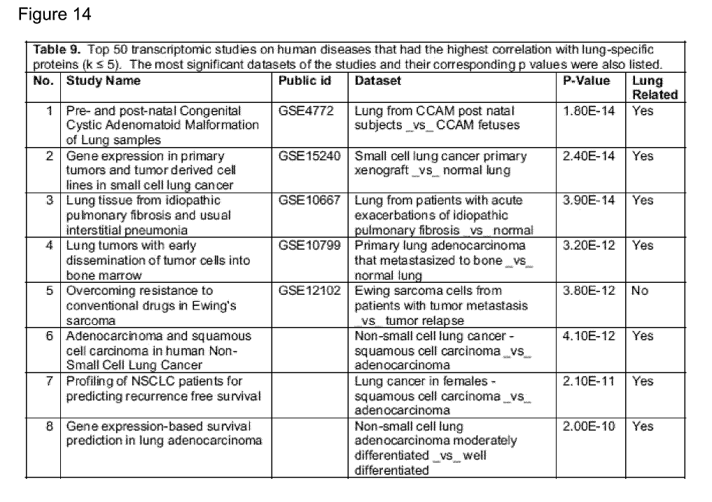

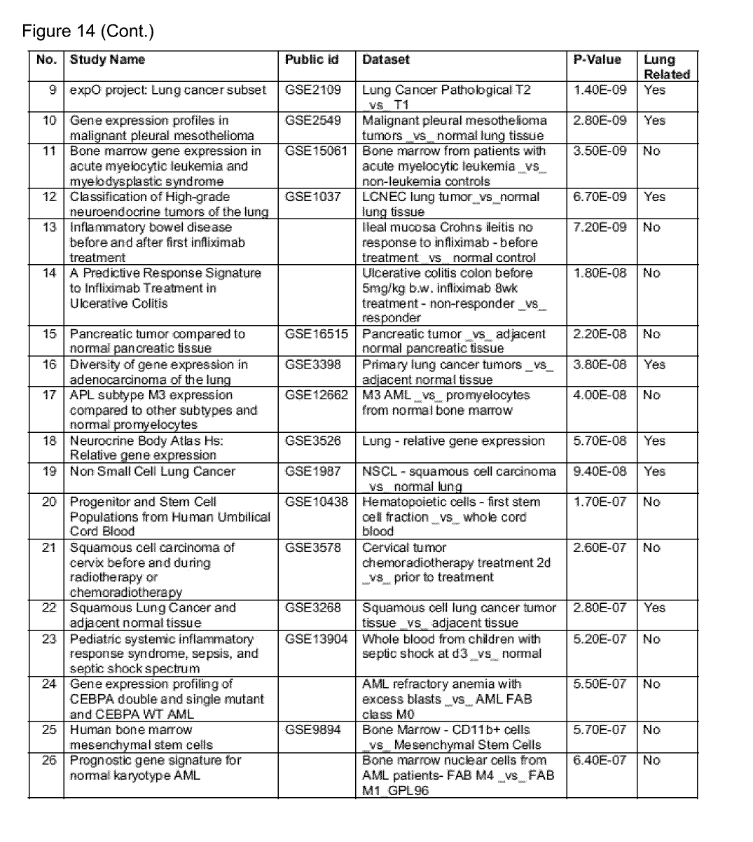

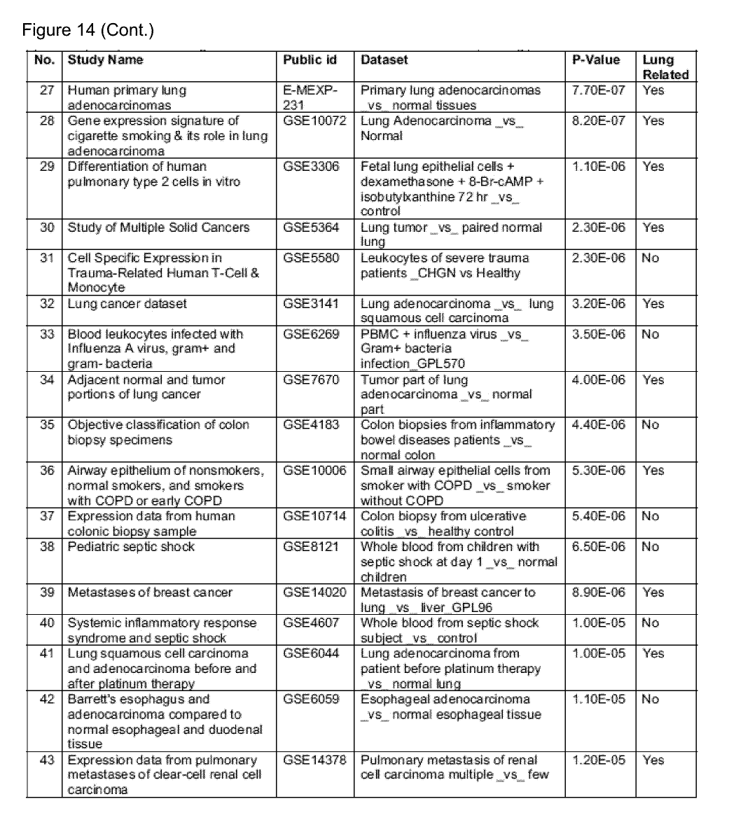

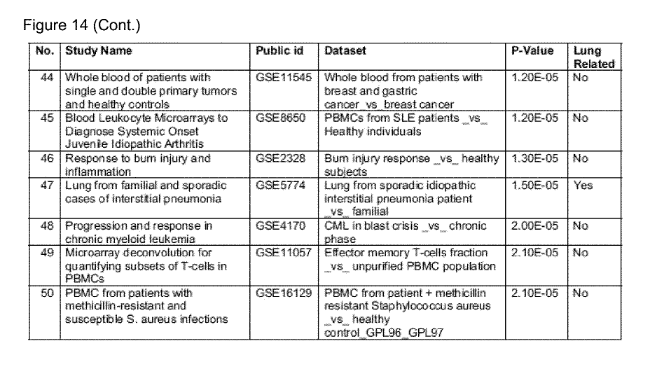

A five-protein organ-specific, lung, panel was identified by selecting five top-ranked lung cancer biomarkers (as described above) that were not most abundant in the organ of lung, but were present in lung. The five proteins developed by comparison of the SBS data set with the Nextbio analysis were CLDN18, CPB2, WIF1, PPBP, and ALOX15B. None of the proteins was lung-specific under conventional definition of organ-specific proteins. As illustrated in FIG. 5, the panel was 100% lung-specific. As discussed above, all five proteins (and thus the panel) were highly indicative for lung cancers. This illustrates that a protein or a panel of proteins that are associated with an organ-associated disease do not need to be specific to that organ alone. A protein or a panel of proteins may be primarily specific to several different organs, yet be highly indicative for a disease in a completely different organ.

### Example 4: Evaluation of Lung-Specific Panels as Biomarkers of Lung Cancer

Lung diseases encompass many disorders affecting the lungs, such as asthma, chronic obstructive pulmonary disease, infections like influenza, pneumonia and tuberculosis, lung cancer, and many other breathing problems. Among cancers, lung cancer is the primary cause of cancer death among both men and women in the U.S. More than 219,000 Americans will be diagnosed with lung cancer (approximately 15 percent of new cancer cases). More than 159,000 will die from the disease, according to the American Cancer Society (2009). Although lung cancer accounts for 15 percent of cancer cases in the United States, it accounts for 28 percent of cancer death as lung cancer typically isn't diagnosed until later and intractable stages, when efficacy of treatment is reduced.

Early detection of lung cancer is difficult since clinical symptoms are often not present until the disease has reached an advanced stage. Currently, diagnosis is aided by the use of chest x-rays, analysis of the type of cells contained in sputum and fiberoptic examination of the bronchial passages. Detection of lung cancer using low-dose computed tomography, (CT) can identify many abnormalities in patients' lungs. Unfortunately, this method has proven to be inefficient as CT scans show abnormalities that are not cancerous. CT scanning produces false positive results for cancer a third of the time. The rate of false positives related to CT scanning is twice the rate of standard X-ray screening and often leads to invasive and potentially harmful follow-up tests including surgery. Treatment regimens are determined by the type and stage of the cancer, and include surgery, radiation therapy and/or chemotherapy.

Early detection of primary, metastatic, and recurrent disease can significantly impact the prognosis of individuals suffering from lung cancer. Non-small cell lung cancer diagnosed at an early stage has a significantly better outcome than when diagnosed at more advanced stages. Similarly, early diagnosis of small cell lung cancer potentially has a better prognosis. Accordingly, there is a great need for more sensitive and accurate assays and methods to measure health and detect disease and monitor treatment at earlier stages.

Using the methods of the invention, panels of lung-specific proteins will be assessed as circulating biomarkers of lung cancer. Markers will be analyzed using large scale Multiple Reaction Monitoring (MRM) assays across cohorts of lung cancer, non-cancerous lung disease and healthy control blood samples.

The panel of markers defined by the SBS data sets that correlate with each of the NextBio clinical studies listed below will be tested. The differentiation of the lung cancer groups by lung spot size is not available on the NextBio data sets, but we anticipate that marker expression levels will be significantly increased or decreased based on degree of stratification of disease.

Samples.

The table below describes the sample cohorts that will be used in a clinical study to evaluate the effectiveness of the lung-specific proteins as biomarkers of lung cancer after detection of a lung spot by imaging. The major cohorts in the study are non-small cell lung cancer (NSCLC) samples and non-cancer groups.

| Major Cohort | Minor Cohort |
|---|---|
| Non-Cancer | Granulomatous Lung Disease |
| Groups | Chronic Obstructive Pulmonary Disease |
| | Chronic Lung Disease (includes IPF) |
| | Normal - Smoker |
| | Normal - Nonsmoker |
| Cancer Groups | Lung Cancer < 10 mm |
| (NSCLC) | Lung Cancer 10 mm to 14 mm |
| | Lung Cancer 15 mm to 19 mm |
| | Lung Cancer 20 mm and larger |
| | Advanced stage lung cancer |
| | Lung cancer with previous cancers |
| | Lymphoma |

The cancer cohort is subdivided by lung spot size (<10 mm, 10 mm to 14 mm, 15 mm to 19 mm and 20 mm or larger). Also included are advanced stage lung cancer (which can present with spots of any size), lung cancer as possible metastasis and lymphoma. It is anticipated that as tumor size gets larger so does the likelihood of detecting a blood-based tumor marker. Hence, the parsing of lung cancer samples by size of spot detected by imaging.

The non-cancer cohort includes confounding lung diseases (granulomatous lung disease, COPD, IPF) that may cause spots to appear on a CT scan or X-ray as well as healthy controls, both smokers and non-smokers.

The samples will be blood samples drawn before tissue confirmation of disease (non-disease) state.

Circulating biomarkers of lung cancer will be able to distinguish samples with lung spots above a certain size (e.g., 10 mm) from non-cancer groups.

Assay Development.

Multiple Reaction Monitoring (MRM) is a mass spectrometry-based assay that enables highly multiplexed assays to be developed rapidly [7]. Depending on assay parameters and mass spectrometric device, up to 100 protein assays can be multiplexed into a single MRM sample analysis [8]. Hundreds of protein assays can be performed on a single blood sample via aliquoting the sample.

MRM assays for all lung-specific panel proteins will be developed. Typically, two peptides and two transitions per peptide will be monitored for each protein giving four data points per assay. Synthetic peptides will be utilized to develop the MRM assays thereby determining peptide retention time and transition masses. Due to the number of proteins (over 100) the protein assays will be grouped into two or three batches for separated MRM runs.

In addition to the lung-specific panel proteins included in the MRM assays, lung-nonspecific markers of lung-cancer and/or lung-disease will be included in the MRM assays. These markers will be obtained from the literature or from proprietary databases. These markers are added as it may be the case that a diagnostic panel for lung cancer includes both lung specific and non-specific markers.

Sample Runs.

Each sample will be divided into 2 or 3 aliquots for MRM runs. Samples will be spiked with peptide standards for normalization of quantification across sample runs. Samples from each cohort will be matched based on clinical data (gender, age, collection site, etc.) and matched samples will be run sequentially through the MRM assays to minimize analytical bias. Protein assay measurements will be obtained for each protein in each sample.

Panel Evaluation.

Due to the large number of protein assays, absolute quantification of each protein will not be determined via labeled peptides because of cost. Instead, normalized relative protein abundance across sample cohorts will be obtained. As the purpose is to verify which lung-specific proteins are blood biomarkers of lung cancer, relative quantification of proteins is sufficient.

For each protein, a statistical test (such as a false discovery rate adjusted one-side paired t-test) will be used to determine if the protein distinguishes cancerous samples above a certain spot size (say, e.g., 10 mm) from non-cancerous samples. Pairing of samples in the statistical test will be determined by the matching of samples as described above. As there are four data points per protein, at least three of the four data points must exhibit a significant statistical difference.

To verify that a specific panel of proteins (either all lung-specific proteins or a particular subset of the lung-specific proteins) is, collectively, a diagnostic panel that distinguishes cancerous samples above a certain spot size (e.g., 10 mm) from non-cancerous samples, the following analysis is performed. All data points for the proteins on the panel are treated as if data points from a single protein and submitted to the paired statistical test. If the false discovery rate adjusted p-value of this test is significant (e.g., below 5%) then the panel is verified as diagnostic. The false discovery rate can be estimated using many methods including permutation testing where the samples from all cohorts are iteratively randomized to provide an estimate of the false discovery rate.

As a final measure, a search strategy to find novel panels of lung specific and/or non-specific markers of lung cancer will be employed. More specifically, let k denote the number of proteins on a proposed diagnostic panel. Let n be the total number of lung specific and non-specific proteins in the MRM assay. For every selection of k proteins from the total number n, perform the diagnostic statistical test described above to determine if that panel of k proteins is diagnostic. This process is repeated for every selection of k proteins. As this process is computing intensive, heuristic search algorithms can be used to search the space of all panels of size k.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

[1] Marioni J C, Mason C E, Mane S M, et. al. RNA-seq: an assessment of technical reproducibility and comparison with gene expression arrays. Genome Res. 2008; 18(9): 1509-17.
[2] Jongeneel C V, Delorenzi M, Iseli C, et. al. An atlas of human gene expression from massively parallel signature sequencing (MPSS). Genome Res. 2005; 15(7): 1007-14.
[3] Stolovitzky G A, Kundaje A, Held G A, et. al. Statistical analysis of MPSS measurements: application to the study of LPS-activated macrophage gene expression. Proc Natl Acad Sci USA. 2005; 102(5): 1402-7.
[4] Bolstad B M, Irizarry R A, Astrand M, Speed T P. A comparison of normalization methods for high density oligonucleotide array data based on variance and bias. Bioinformatics. 2003; 19(2): 185-93.
[5] Su A I, Wiltshire T, Batalov S, et. al. A gene atlas of the mouse and human protein-encoding transcriptomes. Proc Natl Acad Sci USA. 2004; 101(16): 6062-7. i
[6] Hood L, Heath J R, Phelps M E, Lin B. Systems biology and new technologies enable predictive and preventative medicine. Science. 2004; 306(5696): 640-3.
[7] High sensitivity detection of plasma proteins by multiple reaction monitoring of N-glycosites, Stahl-Zeng, Jianru et al., Molecular and Cellular Proteomics, 6 (10), 2007.
[8] High-throughput generation of selected reaction-monitoring assays for proteins and proteomes, Picotti, Paola et al., Nature Methods, 7 (1), 2010.
[9] WO/2008/021290 “ORGAN-SPECIFIC PROTEINS AND METHODS OF THEIR USE”

What is claimed is:

1. A method for predicting a risk for development of a lung disease in a subject, comprising:
determining the protein expression of a plurality of proteins comprising at least CLDN18, CPB2, WIF1, PPBP, and ALOX15B from a biological sample from the subject, wherein said determining step comprises ionizing CLDN18, CPB2, WIF1, PPBP, and ALOX15B;
comparing the protein expression from step (a) to the protein expression of a plurality of proteins comprising at least CLDN18, CPB2, WIF1, PPBP, and ALOX15B from a control biological sample, wherein the control biological sample is obtained from a subject without lung disease;
predicting that the subject is at risk of developing lung disease based on the differential protein expression of the plurality of proteins between the subject biological sample and the control biological sample, wherein the subject is at risk of developing lung disease if the differential protein expression it at least 10%.

2. The method according to claim 1, wherein the lung disease is selected from the group consisting of acute respiratory distress syndrome (ARDS), alpha-1-antitrypsin deficiency, asbestos-related lung diseases, asbestosis, asthma, bronchiectasis, bronchitis, bronchopulmonary dysplasia (BPD), chronic bronchitis, chronic obstructive pulmonary disease (COPD), congenital cystic adenomatoid malformation, cystic fibrosis, emphysema, hemothorax, idiopathic pulmonary fibrosis, infant respiratory distress syndrome, lymphangioleiomyomatosis (LAM), pleural effusion pleurisy and other pleural disorders, pneumonia, pneumonoconiosis, pulmonary arterial hypertension, pulmonary fibrosis, respiratory distress syndrome in infants, sarcoidosis and thoracentesis.

3. The method of claim 1, wherein the lung disease is a lung cancer selected from the group consisting of small cell carcinoma, non-small cell carcinoma, squamous cell carcinoma, adenocarcinoma, broncho-alveolar carcinoma, mixed pulmonary carcinoma, malignant pleural mesothelioma and undifferentiated pulmonary carcinoma.

4. The method of claim 1, wherein protein expression can be determined by mass spectrometry or a multiple-reaction-monitoring mass spectrometry (MRM-MS) assay.

5. The method of claim 4, wherein protein expression can be determined by multiple-reaction-monitoring mass spectrometry (MRM-MS) assay.

6. The method of claim 1, wherein the biological sample is selected from the group consisting of organs, tissue, bodily fluids and cells.

7. The method of claim 6, wherein the bodily fluid is selected from the group consisting of blood, serum, plasma, urine, sputum, saliva, stool, spinal fluid, cerebral spinal fluid, lymph fluid, skin secretions, respiratory secretions, intestinal secretions, genitourinary tract secretions, tears, and milk.


SEQUENCE LISTING (6 sequences)

SEQ ID NO: 1 - DNA, 20 bp, Artificial Sequence
  Artificial Polynucleotide
gatcaaatatcactctccta

SEQ ID NO: 2 - DNA, 20 bp, Artificial Sequence
  Artificial Polynucleotide
gatcaaatatcactctcctc

SEQ ID NO: 3 - DNA, 20 bp, Artificial Sequence
  Artificial Polynucleotide
gatcaaatatcactctcctt

SEQ ID NO: 4 - DNA, 20 bp, Artificial Sequence
  Artificial Polynucleotide
gatcaaatatcactctcctg

SEQ ID NO: 5 - DNA, 20 bp, Artificial Sequence
  Artificial Polynucleotide
gatcaaaaaaacgttctttg

SEQ ID NO: 6 - DNA, 20 bp, Artificial Sequence
  Artificial polynucleotide
gatcaaaaaaaaatttttgc